(19) United States
(12) Patent Application Publication
(10) Pub. No.: US 2024/0407371 A1
(43) Pub. Date: Dec. 12, 2024

(54) METHOD FOR THE KILLING, INACTIVATING, OR INHIBITING OF HARMFUL BLUE-GREEN ALGAE OR ALGAE CAPABLE OF CAUSING HARMFUL ALGAL BLOOM (HAB)

(71) Applicant: Sundew Aps, Copenhagen (DK)

(72) Inventors: Malte Jarlgaard HANSEN, Copenhagen (DK); Foojan MEHRDANA, Copenhagen (DK)

(21) Appl. No.: 18/697,536

(22) PCT Filed: Oct. 10, 2022
(86) PCT No.: PCT/EP2022/078154

§ 371 Date: Apr. 1, 2024

(30) Foreign Application Priority Data
Oct. 11, 2021 (EP) 21202024.2
May 11, 2022 (EP) 22172778.7

(51) Int. Cl. A01N 63/27; A01P 13/00; C12N 1/20; C12R 1/39

(52) U.S. Cl. CPC A01N 63/27; A01P 13/00; C12N 1/20; C12R 2001/39

(57) ABSTRACT

The present invention relates to method for the killing, inactivating, or inhibiting of one or more harmful blue-green algae or algae capable of causing Harmful Algal Bloom (HAB) in marine, brackish or freshwater environments using an effective amount of a lipopeptide biosurfactant.

### TECHNICAL FIELD

The present invention relates to methods for the killing, inactivating, or inhibiting of pathogens or pests using a lipopeptide biosurfactant.

### BACKGROUND

Diseases in agricultural or aquacultural production species, both plants and animals, can be triggered by a multitude of different infectious agents from multicellular parasites to virus particles.

In WO2019101739 it was described how a lipopeptide biosurfactant, obtained from the bacterium Pseudomonas fluorescens strain H6, was used to effectively treat white spot disease in fish caused by ciliate parasite Ichthyophthirius multifiliis (ICH). Previous treatment of ICH included several chemical agents less friendly to the environment such as malachite green, sodium percarbonate, copper sulphate, formalin, peracetic acid and totrazuril. Also, certain plant extracts had attracted attention due to their potential effect on certain life stages of ICH including plant derived compounds comprising cynatratoside-C, sanguinarine, dihydrosanguinarine, dihydrochelerythrine, and pentagalloylglucose. However, the long-term residual impact on the environment, fish, and humans remains unsolved and there is a persistent need for developing new compounds and regimes for the safe treatment of agricultures or aquacultures against infestations.

Controlling of undesired species have been shown previously using various methods.

Liu et al. 2015 discloses that that some Pseudomonas species isolated from healthy salmon eggs produces more biosurfactant than Pseudomonas species isolated from Saprolegnia-infected salmon eggs, and that some Pseudomonas species such as H6 produces a viscosin-like lipopeptide surfactant which inhibits the growth of Saprolegnia diclina on salmon eggs in vitro. However this viscosin-like lipopeptide surfactant offers no significant protection of salmon eggs against Saprolegniosis.

Lamar et al. 2018 discloses control of the scuticociliatosis-causing parasite, Philasterides dicentrarchi using the saposin-like antibacterial peptide Nk-lysin or shortened analogues thereof.

Park Seong Bin et al. 2014 discloses the control of the ciliates that cause scuticociliatosis in olive flounder, including P. dicentrarchi and Miamiensis avidus, using a combination technique involving the disinfectant and surfactant, benzalkonium chloride, and bronopol.

Al-Jubury A et al. 2018 discloses that the Pseudomonas H6 lipopeptide surfactant is able to control the ciliate I. multifilis at various life-cycle stages and suggests its development for application as an antiparasitic control agent in aquaculture.

Jensen Hannah Malene et al. 2020 discloses that the Pseudomonas H6 lipopeptide surfactant is able to control gill amoebae in freshwater rainbow trout.

Parama et al. 2007 discloses the effects of the cysteine proteinases isolated from P. dicentrarc on the phagocytic functions of turbot pronephric leucocytes. Further, Parama et al. 2007 teaches that the pro-inflammatory cytokine IL-1 beta is expressed in fish infected with P. dicentrarchi like those infected with other ciliate parasites, I. multifiliis or the monogenean Gyrodactylus derjavin.

However, it remains unknown in the art which mechanism-of-action the lipopeptide biosurfactant of the art excerpts on Ichthyophthirius multifiliis or Saprolegnia diclina, which treatment regimes can be used to treat these pests in live fish in aquaculture and if lipopeptide biosurfactant of the art is effective against other types of pathogen pests sharing little or no commonalities. For example Saprolegnia is an oomycete mold which differs significantly in genotype, phenotype, habitat, life-cycle and reproduction from Ichthyophthirius which is a Ciliate.

Many microbial and simple pathogens or pests having short life spans and generation times are evolutionary much further apart compared with longer living organisms, such as humans. While humans have a generation time of approximately 25 years, many microbial or simple organisms can have generation times of days, weeks, or months, and therefore evolutionary divergence of microbes and simple organisms happens much faster than for higher life forms. In addition the anthropocentric structure of taxonomic classification creates finer divisions between taxonomic groups the more closely related they are to humans, whilst conversely grouping those that are unrelated. Accordingly, while e.g. Ichthyophthrius and Tetrahymena are both single cell organisms, they are in fact extremely biologically and genetically divergent, and the methodology for controlling such divergent species is unpredictable a priori of any known established mechanism of action.

### SUMMARY

Against this background art, the inventors of the present invention have now found that a lipopeptide surfactant, such as the lipopeptide surfactant from Pseudomonas fluorescens strain DSMZ-34058, can effectively be used to kill, inactivate, or inhibit a range of pathogens or pests or combination of pathogen or pests, which causes disease or poisoning upon infecting production species in agriculture and aquaculture and accordingly, the invention disclosed herein provides for a method for the killing, inactivating, or inhibiting of one or more pathogens or pests selected from
a) Ciliates, which is not Ichthyophthirius multifiliis' or Cryptocaryon irritans;
b) Flagellated protists, including dinoflagellates;
c) Flatworms;
d) Amoebae;
e) Bacteria; including cyanobacteria
f) Viruses;
g) Oomycetes, which is not Saprolegnia diclina; and/or
h) Fungi;
   comprising contacting the pathogen or pest with an effective amount of a lipopeptide biosurfactant.

Moreover, it has now been been found that the killing effect on Ichthyophthrius seems to be associated with effects on cilia as well on the membrane which mechanism of action would differ completely the MoA on Saprolegnia because the latter is an oomycete which do not possess cilia.

In some aspects, the present disclosure provides for a method for the killing, inactivating, or inhibiting of one or more pathogens or pests selected from
a) Ciliates, which is not Ichthyophthirius multifiliis' or Cryptocaryon irritans;
b) Flagellated protists, including dinoflagellates;
c) Sporozoans;
d) Bacteria; including cyanobacteria
e) Viruses;
f) Oomycetes, which is not Saprolegnia diclina; and/or
g) Fungi;
   comprising contacting the pathogen or pest with an effective amount of a lipopeptide biosurfactant.

Surprisingly, it has been shown that the lipopeptide surfactant is effective in natural habitat of these pathogens and in production species of agriculture and aquaculture, using an improved dosing regime performing better that the known pesticides such as formalin and/or Cu based pesticides currently used in agriculture and aquaculture and moreover the lipopeptide biosurfactant is biologically degradable in nature and much less toxic damaging to the ecology of the habitat.

In a further aspect as described herein is a lipopeptide biosurfactant for use in the treatment of an infection in a subject by one or more pathogens or pests selected from
a) Ciliates, which is not Ichthyophthirius multifiliis or Cryptocaryon irritans;
b) Flagellated protists, including dinoflagellates;
c) Flatworms;
d) Amoebae
e) Bacteria, including cyanobacteria;
f) Viruses;
g) Oomycetes; and/or
h) Fungi.

In a further aspect as described herein is a lipopeptide biosurfactant for use in the treatment of an infection in a subject by one or more pathogens or pests selected from
a) Ciliates, which is not Ichthyophthirius multifiliis or Cryptocaryon irritans;
b) Flagellated protists, including dinoflagellates;
c) Sporozoans;
d) Bacteria, including cyanobacteria;
e) Viruses;
f) Oomycetes; and/or
g) Fungi.

In a further aspect disclosed herein is a bacterial isolate for use in the treatment of an infection in a subject by one or more pathogens or pests selected from
a) Ciliates, which is not Ichthyophthirius multifiliis or Cryptocaryon irritans;
b) Flagellated protists, including dinoflagellates;
c) Flatworms;
d) Amoebae;
e) Bacteria, including cyanobacteria;
f) Viruses;
g) Oomycetes, which is not Saprolegnia diclina; and/or
h) Fungi,
   in a target organism susceptible to said infection, wherein the bacterial isolate comprises bacteria that produce a lipopeptide surfactant.

In a further aspect disclosed herein is a bacterial isolate for use in the treatment of an infection in a subject by one or more pathogens or pests selected from
i) Ciliates, which is not Ichthyophthirius multifiliis or Cryptocaryon irritans;
j) Flagellated protists, including dinoflagellates;
k) Sporozoans;
l) Bacteria, including cyanobacteria;
m) Viruses;
n) Oomycetes, which is not Saprolegnia diclina; and/or
o) Fungi,
   in a target organism susceptible to said infection, wherein the bacterial isolate comprises bacteria that produce a lipopeptide surfactant.

### DESCRIPTION OF DRAWINGS AND FIGURES

The figures included herein are illustrative and simplified for clarity, and they merely show details which are essential to the understanding of the invention, while other details may have been left out. Throughout the specification, claims and drawings the same reference numerals are used for identical or corresponding parts. In the figures and drawing include herein:

FIG. 1 shows a HPLC chromatogram of a lipopeptide biosurfactant isolate of a fermentate of Pseudomonas sp., strain LMG 5329.

FIG. 2 shows a HPLC chromatogram of a lipopeptide biosurfactant isolate of a fermentate of Pseudomonas fluorescens strain H6 deposited under CBS 143505.

FIG. 3 shows the time course of in vivo chlorophyll fluorescence (A) and cell abundance (B) and Alexandrium tamarense (C, D).

FIG. 4 shows the time course of in vivo chlorophyll fluorescence (A) and cell abundance (B) for Aureococcus anophagefferens (CCMP1984).

FIG. 5 shows the time course of cell abundance for Gambierdiscus toxicus (CCMP3466).

FIG. 6 shows the time course of in vivo chlorophyll fluorescence (A) and cell abundance (B) for Heterosigma akashiwo (CCMP3149).

FIG. 7 shows the time course of in vivo chlorophyll fluorescence (A) and cell abundance (B) for Karenia brevis (CCMP2281).

FIG. 8 shows the time course of in vivo chlorophyll fluorescence (A) and cell abundance (B) for Prymnesium parvum (CCMP3037).

FIG. 9 shows the time course of in vivo chlorophyll fluorescence (A) and cell abundance (B) of Nostoc sp. (CCMP3413).

FIG. 10 shows the time course of in vivo chlorophyll fluorescence (A) and cell abundance (B) for Aphanizomenon sp. (CCMP2764).

FIG. 11 shows the time course of in vivo chlorophyll fluorescence (A) and cell abundance (B) for Microcystis cf. aeruginosa (CCM P3462).

FIG. 12 shows the time course of in vivo chlorophyll fluorescence (A) and cell abundance (B) for Alexandrium tamarense (CCMP1771).

FIG. 13 shows the time course of in vivo chlorophyll fluorescence (A) and cell abundance (B) for Heterosigma akashiwo (CCMP3149).

FIG. 14 shows the time course of in vivo chlorophyll fluorescence (A) and cell abundance (B) for Karenia brevis (CCMP2281).

FIG. 15 shows the time course of in vivo chlorophyll fluorescence (A) and cell abundance (B) for Prymnesium parvum (CCMP3037).

FIG. 16 shows the time course of in vivo chlorophyll fluorescence (A) and cell abundance (B) for Prorocentrum lima (CCM P684).

FIG. 17 shows the time course of in vivo chlorophyll fluorescence (A) and cell abundance (B) for Chattonella marina (CCM P2962).

FIG. 18 shows the mean percentage of sporulate Eimeria oocysts obtained from Example 16.

FIG. 19 shows the mean percentage of excysted Cryptosporidium parvum oocysts obtained from Example 16.

FIG. 20 shows the effect of LP34058 at 6.3, 12.6, 18.9, 25.2, 31.5 and 37.8 mg active compound/L on the survival of T. thermophila at various time points up to 60 min.

FIG. 21 shows the effect of LP34058 at 6.3, 12.6, 18.9, 25.2, 31.5 and 37.8 mg active compound/L on the survival of T. pyriformis at various time points up to 60 min.

FIG. 22 shows the effect of lipopeptide surfactant LP34058 on Phytophthora ramorum at various time points.

FIG. 23 shows the effect of lipopeptide surfactant LP34058 on Phytophthora cryptogea at various time points.

FIG. 24 shows the effect of lipopeptide surfactant LP34058 on Fusarium oxysporum sp. Gladioli at various time points.

FIG. 25 shows the effect of lipopeptide surfactant LP34058 on Fusarium oxysporum f.sp. lycopersici at various time points.

FIG. 26 shows the effect of lipopeptide surfactant LP34058 on Verticillium dahliae at various time points.

FIG. 27 shows the effect of lipopeptide surfactant LP34058 on Aphanomyces astaci at various time points.

FIG. 28 shows the effect of lipopeptide surfactant LP34058 on Aphanomyces species at various time points.

FIG. 29 shows the effect of lipopeptide surfactant LP34058 on Saprolegnia parasitica at various time points.

FIG. 30 shows the effect of lipopeptide surfactant LP34058 on Pythium catenulatum at various time points.

FIG. 30 shows the effect of lipopeptide surfactant LP34058 on Pythium catenulatum at various time points.

FIG. 31 shows the effect of lipopeptide surfactant LP34058 on Pythium dissotocum at various time points.

FIG. 32 shows the mean number of G. lamblia cysts counted in 5.1 μl of sample obtained in Example 20.

FIG. 33 shows the mean number of G. lamblia cysts counted in 20.1 μl of sample obtained in Example 20 repeat assay.

FIG. 34 shows the trojan preparation of Example 23 by placing heat killed eggs on S. diclina culture plates and method of pathogen inoculation.

FIG. 35 shows the state of the eggs in different treatments at termination. Letters on the images represent the treatment details. Numbers on each photograph represent replications. Black arrows in each well refer to pre-colonized trojan 10 days post inoculation. The mycelia spread from trojan to nearby eggs giving a smear impression around trojan. The white arrows refer to the dead eggs recognized by it complete white out color and opaqueness. Live eggs were distinguished by the bright darker color and in many cases movement of developing embryo inside.

FIG. 36 shows a comparison of the life cycles for Ich (a) and Tetrahymena (b), respectively.

FIG. 37 shows A) Percent of entanglement with mycelia and attachment to trojans in different treatments; Bars of treatment E (Formalin) and G (No Sap-Control) are not shown here because of 0 (zero) entanglement; B) Percent of eggs appeared alive at termination in different treatments. The numbers also include entangled eggs to trojan that looked dying but not whiteout dead; C) Completely whiteout dead eggs and attached to trojan in different treatments; D) Number of hatched eggs in different treatments. Letters above shows statistically significant differences between treatments. Treatments sharing same letters do not have significant differences.

FIG. 38 shows A) Percent of eggs entanglement with mycelia and attachment to trojans in different treatments; Bars of treatment M (No Sap-Control) is not shown here because of 0 (zero) entanglement; B) Percent of eggs appeared alive at termination in different treatments; C) Completely whiteout dead eggs and attached to trojan in different treatments; D) Number of hatched eggs in different treatments. Letters above shows statistically significant differences between treatments. Treatments sharing letters do not have significant differences.

FIG. 39 shows the impact of lipopeptide biosurfactant of LP34058 on biofilter bacteria presented with the concentrations of TAN, nitrite, and nitrate in Example 8.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

The features and advantages of the present invention is readily apparent to a person skilled in the art by the below detailed description of embodiments and examples of the invention with reference to the figures and drawings included herein.

### Definitions

The terms “substantially” or “approximately” or “about”, if used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

The term “freshwater fish” as used herein refers to fish living at least during a certain stage of its life cycle in freshwater. Example of freshwater fish includes salmonids (e.g. rainbow trout (Oncorhynchus mykiss), aquaculture (such as salmonids (exemplified by rainbow trout (Oncorhynchus mykiss), cyprinids (e.g. grass carp (Ctenopharyngodon idella), black carp (e.g. Mylopharyngodon piceus), silver carp (Hypophthalmichthys molitrix), common carp (Cyprinus carpio), bighead carp (Hypophthalmichthys nobilis), catla (Indian carp, Catla catla), crucian carp (Carassius carassius), roho labeo (Labeo rohita)), tilapia (e.g. Nile tilapia (Oreochromis niloticus)), milkfish (Chanos chanos), catfish (e.g. Amur catfish (Silurus asotus)), Wuchang bream (Megalobrama amblycephala), northern snakehead (Channa argus).

The term “marine fish” as used herein refers to fish species living at least a part of their life in marine waters. Examples are fish raised for aquaculture in mariculture systems such as gilthead seabream (Sparus auratus) and seabass (Dicentrarchus labrax). In addition, a long range of ornamental fish species used in marine aquaria is covered by the term.

The term “lipopeptide biosurfactant” as used herein refers to a compound or composition of compounds comprising a lipid connected to a peptide, preferably a cyclic peptide having surfactant properties. Surfactant properties are to be understood as lowering the surface tension (or interfacial tension) between two liquids, between a gas and a liquid, or between a liquid and a solid. The lipopeptide biosurfactant of the invention is preferably an organic compound or composition of organic compounds that are amphiphilic.

The term “in vitro” as used herein refers to medical procedures, tests, and experiments that are performed outside of a living organism. An in vitro study occurs in a controlled environment, such as a test tube or petri dish.

The term “in vivo” as used herein refers to tests, experiments, and procedures that are performed in or on a whole living organism, such as a person, laboratory animal, or plant.

All methods described herein can be performed in any suitable order of steps unless otherwise indicated herein or otherwise clearly contradicted by context. The use of any and all examples, or exemplary language (e.g., “such as”) provided herein is intended merely to better illustrate the invention and does not pose a limitation on the scope of the invention otherwise claimed. No language in the specification should be construed as indicating any non-claimed element essential to the practice of the invention.

The term “Cyanobacteria” as used herein are also referred to as blue-green algae. Blue-green algae are a type of prokaryotes that obtain energy via photosynthesis. The main difference between bacteria and cyanobacteria/blue-green algae is that the bacteria are mainly heterotrophs while the cyanobacteria are autotrophs. Furthermore, bacteria do not contain chlorophyll while blue-green algae contain chlorophyll-a.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

The present invention provides for methods for the killing, inactivating, or inhibiting of one or more pathogens or pests selected from
a) Ciliates, which is not Ichthyophthirius multifiliis or Cryptocaryon irritans;
b) Flagellated protists, including dinoflagellates;
c) Flatworms;
d) Amoebae;
e) Bacteria, including cyanobacteria;
f) Viruses;
g) Oomycetes, which is not Saprolegnia diclina; and/or
h) Fungi; comprising contacting the pathogen or pest with an effective amount of a lipopeptide biosurfactant.

The present disclosure also provides for methods for the killing, inactivating, or inhibiting of one or more pathogens or pests selected from
i) Ciliates, which is not Ichthyophthirius multifiliis or Cryptocaryon irritans;
j) Flagellated protists, including dinoflagellates;
k) Sporozoans;
l) Bacteria, including cyanobacteria;
m) Viruses;
n) Oomycetes, which is not Saprolegnia diclina; and/or
o) Fungi;
   comprising contacting the pathogen or pest with an effective amount of a lipopeptide biosurfactant.

The lipopeptide biosurfactant of the invention can be any lipopeptide surfactant compound or composition of compounds that procides treatment of the pathogens or pests selected herein. Suitable lipopeptide surfactants include surfactin and derivatives thereof, daptomycin and derivatives thereof, massetolide and derivatives thereof, viscosin and derivatives thereof, thanamycin and derivatives thereof and putisolvin and derivatives thereof. In one embodiment the lipopeptide biosurfactant of the invention is a) a viscosin or viscosin-like lipopeptide or a derivative thereof; b) a massetolide or a derivative thereof and/or c) a putisolvin or a derivative or any combination of the foregoing.

Viscosin-like lipopeptides are lipopeptides that are structurally and/or functionally similar to viscosin (IUPAC: (4R)-5-[[(3S,6R,9S,12R,15S,18R,21R,22R)-3-[(2S)-butan-2-yl]-6,12-bis(hydroxymethyl)-22-methyl-9,15-bis(2-methylpropyl)-2,5,8,11,14,17,20-heptaoxo-18-propan-2-yl-1-oxa-4,7,10,13,16,19-hexazacyclodocos-21-yl]amino]-4-[[(2S)-2-[[(3R)-3-hydroxydecanoyl]amino]-4-methylpentanoyl]amino]-5-oxopentanoic acid).

In some embodiments, the viscosin-like lipopeptide comprises, similarly to viscosin, a circular peptide and a fatty acid covalently connected to the circular peptide. In some embodiments, the viscosin-like lipopeptide comprises 9 amino acids according to the general formula: L-Leucine-X1-D-Allothreonine-X2-X3-D-Serine-L-Leucine-D-Serine-X4; wherein any one of X1, X2, X3, and X4 can be any amino acid independently of each other, such as any proteinogenic amino acid, but in some embodiments;
“X1” is D-Glutamate or D-Glutamine;
“X2” is D-Valine, D-Isoleucine or D-Alloisoleucine;
“X3” is L-Leucine or D-Leucine; and
“X4” is L-Isoleucine, L-Leucine or L-Valine,
in any combination of X1, X2, X3, and X4. In some embodiments, the 9 amino acids are cyclized.

In some embodiments, X1 is D-Glutamate and X2-X4 are as defined according to the general formula above.

In some embodiments, X1 is D-Glutamine and X2-X4 are as defined according to the general formula above.

In some embodiments, X2 is D-Valine and X1, X3-X4 are as defined according to the general formula above.

In some embodiments, X2 is D-Isoleucine and X1, X3-X4 are as defined according to the general formula above.

In some embodiments, X2 is D-Alloisoleucine and X1, X3-X4 are as defined according to the general formula above.

In some embodiments, X3 is L-Leucine and X1-X2, X4 are as defined according to the general formula above.

In some embodiments, X3 is D-Leucine and X1-X2, X4 are as defined according to the general formula above.

In some embodiments, X4 is L-Isoleucine and X1-X3 are as defined according to the general formula above.

In some embodiments, X4 is L-Leucine and X1-X3 are as defined according to the general formula above.

In some embodiments, X4 is L-Valine and X1-X3 are as defined according to the general formula above.

In some embodiments, the viscosin-like lipopeptide comprises 9 amino acids being 3×leucine, 2×serine, valine, threonine, isoleucine, and glutamic acid. In some embodiments, the viscosin-like lipopeptide comprises 9 amino acids being 3×leucine, 2×serine, valine, threonine, isoleucine, and glutamic acid.

In some embodiments, the hydroxyl group of the D-Allothreonine is bound to the carboxyl group of the amino acid residue defined by “X4”, such as to form an ester. In some embodiments, the D-Allothreonine is bound to X4 via its hydroxyl group of its side chain.

In some embodiments, the L-Leucine bound to X1 in the general formula described above is covalently connected to a fatty acid. In some embodiments, the fatty acid is C3-hydroxylated. In some embodiments, the fatty acid has a length of from 10 to 16 carbon atoms, such as from 10 to 11, such as from 11 to 12, such as from 12 to 13, such as from 13 to 14, such as from 14 to 15, such as from 15 to 16 carbon atoms. In some embodiments, the fatty acid is C3-hydroxylated and has a length of from 10 to 16 carbon atoms. In some embodiments, the fatty acid is 3-OH-decanoic acid. In preferred embodiments, the fatty acid is covalently attached via its carboxyl functionality.

In some embodiments, the viscosin-like lipopeptide of the present disclosure has a molecular weight of from 1111 to 1168 g/mol. In some embodiments, the viscosin-like lipopeptide has a molecular weight of from 1111 to 1168 g/mol and comprises the general formula as described above.

In some embodiments, the viscosin-like lipopeptide has a molecular weight of from 1111 to 1168 g/mol and comprises the general formula as described above, wherein L-leucine which is connected to X1 is also connected to a fatty acid, for example a fatty acid as disclosed herein, which is optionally C3-hydroxylated.

In some embodiments, the viscosin-like lipopeptide of the present disclosure is selected from the group consistinf of: Viscosin, Massetolide A, B, C, D, E, F, G, and H, WLIP (“White Line Inducing Principle”, Pseudophomin A and B, Viscosinamide A, B, C, and D, and Pseudodesmin A and B.

In some embodiments, the present disclosure provides a combination of one or more viscosin-like lipopeptides as disclosed herein. In such embodiments, the lipopeptide biosurfactant comprises one or more of said viscosin-like lipopeptides, optionally in a predefined ratio.

In some embodiments, the lipopeptide surfactant is provided in a composition comprising a combination of one or more viscosin-like lipopeptides as defined herein, wherein a single viscosin-like lipopeptide as defined herein accounts for at least 50% of the total combination of viscosin-like lipopeptides, such as from 50% to 99% of the total combination of viscosin-like lipopeptides, such as from 50% to 60%, such as from 60% to 70%, such as from 70% to 80%, such as from 80% to 90%, such as from 90% to 95%, such as from 95% to 99%.

In some embodiments, the single viscosin-like lipopeptide accounting for at least 50% of the total combination of viscosin-like lipopeptides comprises the general formula as described above, wherein L-leucine which is connected to X1 is also connected to a fatty acid, for example a fatty acid as disclosed herein, which is optionally C3-hydroxylated.

In some embodiment the lipopeptide surfactant is derived from a microbial source, such as a bacterium, a fungus or an achaea. Generally, the biosynthetic pathway encoding the lipopeptide surfactant within a given microbial strain leads to a single main lipopeptide surfactant and minor amounts of structurally related derivatives of the main lipopeptide surfactant. Useful and known bacterial lipopeptide biosurfactants includes for example massetolide A, massetolide B, massetolide C, massetolide D, massetolide E, massetolide F, massetolide G and massetolide H. Other useful and known bacterial lipopeptide biosurfactants includes putisolvin I and putisolvin II.

A further particularly useful viscosin-like lipopeptide biosurfactant is produced by and obtainable from the bacterium Pseudomonas fluorescens strain H6. Pseudomonas fluorescens strain H6 is also described in Liu et al. 2015 and a sample of the Pseudomonas fluorescens strain H6 was deposited on Nov. 1, 2017 under the Regulations of the Budapest Treaty in the CBS collection of the Westerdijk Fungal Biodiversity Institute with deposit number CBS 143505, which deposit is available by reference in the WO2019101739.

A further particularly useful viscosin-like lipopeptide biosurfactant is produced by and obtainable from the bacterium Pseudomonas fluorescens strain SDW1. A sample of the Pseudomonas fluorescens strain SDW1 was deposited on 6 Oct. 2021 under the Regulations of the Budapest Treaty in the DSMZ—German Collection of Microorganisms and Cell Cultures GmbH under deposit number DSMZ-34058.

In a particular embodiment the lipopeptide surfactant indeed comprises a viscosin or viscosin-like lipopeptide isolated from Pseudomonas fluorescens strain H6 or SDW1. In other embodiments lipopeptide biosurfactant of the invention comprises a massetolide, such as a massetolide lipopeptide surfactant obtainable from Pseudomonas fluorescens strain SS101 or a derivative thereof. In addition or in the alternative, the lipopeptide biosurfactant of the invention can also comprise a putisolvin, such as the putisolvin biosurfactant obtainable from Pseudomonas putida 267 or a derivative thereof. In addition or in the alternative, the lipopeptide biosurfactant of the invention can also comprise a viscosin lipopeptide, such as the viscosin lipopeptide obtainable from Pseudomonas fluorescens SBW25 or a derivative thereof. The isolation and characterization of the lipopeptide biosurfactant of Pseudomonas fluorescens strain H6 or SDW1 can be done as described by Liu et al. (2015) and is found to be clearly distinguished from the well-known lipopeptide biosurfactants of related strains such as the massetolide lipopeptide obtained from Pseudomonas fluorescens SS101 (described in De Bruijn et al (2008)), the viscosin lipopeptide obtained from Pseudomonas fluorescens SBW25 and the putisolvin lipopeptide obtained from Pseudomonas putida 267 (described in Kruijt et al (2008)).

The lipopeptide biosurfactant may be incorporated into a composition comprising a single lipopeptide biosurfactant compound or two or more lipopeptide compounds. Such compostion can further include one or more carriers, agents, adjuvants, additives and/or excipients. The composition of the invention can further be formulated into a desirable final dry or liquid formula such as a slow-release form capable of releasing the lipopeptide biosurfactant(s) over a prolonged period of time to a surrounding medium. Suitable formulations include spray dried, lyophilized, granulated extruded, liquid stabilized formulas,

In particular embodiments the lipopeptide biosurfactant is isolated from a microbial source such as strains of Pseudomonas including Pseudomonas fluorescens strain H6 or SDW1, Pseudomonas fluorescens strain SS101, Pseudomonas fluorescens strain SBW25 and/or Pseudomons putida strain 267, as a composition which contain only a limited amount of ammonia, such as below 10 mg ammonia/g LS. Ammonia, can advantageously be used to promote microbial production of the lipopeptide biosurfactant, but is undesirable in composition to be used for treatment of infections in subject because ammonia generally is toxic to fish.

In some cases, the pathogen or pest is contacted with the lipopeptide biosurfactant in an aqueous solution comprising an effective amount of the lipopeptide biosurfactant to kill, inactivate, or inhibit the pathogen or pest. Such aqueous solutions can be is a hypersaline (hyper saline lakes or trapped tidal coastal water bodies), a marine (sea water), a brackish (mixture of sea water and fresh water eg from rivers) or a freshwater solution (waters of lakes, rivers, bogs, puddles etc.).

In some cases, the pathogen or pest is a flagellated protist including dinoflagellate. Flagellated protists, particularly in the form of microalgae species can given the right conditions grow excessively in hypersaline, marine, brackish and/or freshwater environments known as a Harmful Algal Bloom (HAB), also for marine environments known as “red tide”. HAB is an algal bloom that causes negative impacts to other organisms including humans via production of natural algae-produced toxins, mechanical damage to other organisms, or by other means. HABs are sometimes defined as only those algal blooms that produce toxins, and sometimes as any algal bloom that can result in severely lower oxygen levels in natural waters, killing organisms in marine or fresh waters. More specifically, species causing HAB usually have one or more of the following properties and harmful impacts: i) non-toxic species but high biomass blooming species that can directly or indirectly kill marine organisms by deoxygenation of water bodies, or by their physical effects, ii) species producing toxins involved in food poisoning in humans with either neurological or gastrointestinal symptoms, iii) species causing no damage to humans but which are harmful to fish and marine invertebrates by mechanical effects. Such blooms can last from a few days to many months. After the bloom dies, the microbes that decompose the dead algae use up more of the oxygen, generating a “dead zone” which can cause fish die-offs. When these zones can cover a large area for an extended period of time, where neither fish nor plants are able to survive.

Described herein is an environment friendly solution to the problem of Harmful Algal Bloom, using natural fermented compounds killing, inactivating, or inhibiting the microalgaes causing the HAB. In a separate aspect the solution is a method for the killing, inactivating, or inhibiting of one or more flagellated protists comprising contacting the flagellated protists in an aqueous solution with an effective amount of a lipopeptide biosurfactant. The lipopeptide biosurfactants described herein is less toxic than the toxins naturally produced by the HAB microalgae.

In some embodiments the aqueous solution is a hypersaline solution, a marine solution, a brackish solution or a freshwater solution, and the flagellated protist is a micro algae, optionally a harmful microalgae capable of causing Harmful Algal Bloom (HAB) in hypersaline, marine solution, brackish or freshwater environments. The flagellated protist is suitably selected from one or more of the classes Dinophyceae, Pelagophyceae, Raphidophyceae, or Prymnesiophyceae. The Dinophyceae is suitably of the order Actiniscales, Akashiwales, Amphilothales, Apodiniales, Blastodiniales, Brachidiniales, Dinophysales, Dinotrichales, Gonyaulacales, Gymnodiniales, Haplozoonales, Nannoceratopsiales, Peridiniales, Phytodiniales, Prorocentrales, Ptychodiscales, orThoracosphaerales or a combination thereof. Within this embodiment the Gonyaulacales can be of the family Ostreopsidaceae, optionally of the genus Alexandrium, optionally of the species A. tamarense; and/or optionally of the genus Gambierdiscus, optionally the species G. toxicus. Additionally or alternatively, the Gymnodiniales can be of the family Kareniaceae; optionally of the genus Karenia; optiojally of the species K. brevis. The Pelagophyceae can be of the order Pelagomonadales or Sarcinochrysidales or a combination thereof, where the Pelagomonadales suitably is of the genus Aureococcus, optionally the species A. anophagefferens. The Raphidophyceae can be of the order Actinophryida, Chattonellales, Commatiida, or Raphidomonadales or a combination thereof, where the Chattonellales suitably is of the genus Heterosigma, optionally the species H. akashiwo. The Prymnesiophyceae can be of the order Coccolithales, Coccosphaerales, Isochrysidales, Phaeocystales, Prymnesiales, Syracosphaerales, or Zygodiscales, where the Prymnesiales suitably is of the genus Prymnesium, optionally the species P. parvum.

In some embodiments the aqueous solution is freshwater, and the pathogen or pest is:
a) a ciliate parasite selected from the genus Trichodina causing the disease trichodiniasis;
b) a ciliate parasite selected from the genus Chilodonella causing the disease chilodonellosis;
c) a ciliate parasite selected from the genus Tetrahymena causing the disease guppy disease;
d) a flagellated protist selected from the genus Ichthyobodo (Costia) causing the disease ichthyobodiasis (costiasis);
e) a flatworm selected from the genus Gyrodactylus causing the disease gyrodactylosis;
f) a flatworm selected from the genus Dactylogyrus causing the disease dactylogyrosis;
g) a bacterium selected from the genus Aeromonas causing the diseases furunculosis or tail rot or fin rot or enteritis or hemorrhagic septicemia;
h) a bacterium selected from the genus Flavobacterium causing the disease bacterial gill disease/rainbow trout fry syndrome/columnaris;
i) a bacterium selected from the genus Streptococcus causing the disease streptococcosis; and/or
j) an oomycete selected from i) the genus Achlya causing the disease saprolegniasis and/or ii) the genus Aphanomyces causing the disease epizootic ulcerative syndrome (EUS)/crayfish plague.

In some embodiments the aqueous solution is freshwater and the pathogen or pest is:
a) a ciliate parasite selected from the genus Chilodonella causing the disease chilodonellosis;
b) a ciliate parasite selected from the genus Tetrahymena causing the disease guppy disease;
c) a flagellated protist selected from the genus Ichthyobodo (Costia) causing the disease ichthyobodiasis (costiasis);
d) a bacterium selected from the genus Aeromonas causing the diseases furunculosis or tail rot or fin rot or enteritis or hemorrhagic septicemia;
e) a bacterium selected from the genus Flavobacterium causing the disease bacterial gill disease/rainbow trout fry syndrome/columnaris;
f) a bacterium selected from the genus Streptococcus causing the disease streptococcosis; and/or
g) an oomycete selected from i) the genus Aphanomyces causing the disease epizootic ulcerative syndrome (EUS)/crayfish plague.

In other embodiments the aqueous solution is hypersaline, marine, or brackish marine and the pathogen or pest is
a) a dinoflagellate parasite selected from the genus Amyloodinium causing the disease marine velvet;
b) a flatworm selected from the genus Sparicotyle causing the disease sparicotylosis;
c) an amoebae selected from the genus Neoparamoeba causing the disease amoebic gill disease;
d) a bacterium selected from the genus Photobacterium causing the disease pseudotuberculosis/fish pasteurellosis;
e) a bacterium selected from the genus Vibrio causing the disease vibriosis; and/or
f) a virus selected from the genus Novirhabdovirus causing the disease viral hemorrhagic septicemia (VHS)/infectious hematopoietic necrosis (IHN).

In other embodiments the aqueous solution is hypersaline, marine, or brackish marine and the pathogen or pest is
a) a bacterium selected from the genus Photobacterium causing the disease pseudotuberculosis/fish pasteurellosis;
b) a bacterium selected from the genus Vibrio causing the disease vibriosis; and/or
c) a virus selected from the genus Novirhabdovirus causing the disease viral hemorrhagic septicemia (VHS).

In further embodiments the pathogen or pest is causing disease in mollusks and the pathogen or pest is:
a) a dinoflagellate parasite selected from the genus Perkinsus;
b) a bacterium selected from the genus Nocardia causing the disease pacific oyster nocardiosis; and/or
c) an oomycete selected from the genus Halioticida causing the disease abalone tubercle mycosis.

In still other cases the pathogen or pest is a virus selected from the genus Whispovirus causing white spot syndrome in whiteleg shrimp

Additionally or alternatively the pathogen or pest can also be a plant pathogen or pest, such as
a) a bacterium selected from the genus Candidatus causing the disease citrus greening disease;
b) an oomycete selected from the genus Pythium causing the disease damping off;
c) an oomycete selected from the genus Phytophthora causing the disease potato late blight/Phytophthora blight/root & stem rot/downy mildew/black shank; and/or
d) a fungus selected from i) the genus Fusarium causing the disease Fusarium wilt/Panama disease, ii) the genus Verticillium causing the disease verticillium wilt, iii) the genus Rhizoctonia causing the disease root rot, and/or iv) the genus Botrytis causing the disease gray mold.

Additionally or alternatively the pathogen or pest can also bea human pathogen or pest such as a flatworm selected from the genus Schistosoma causing the disease schistosomiasis in humans and wherein the pathogen or pest is contacted with the lipopeptide biosurfactant in an amount effective of preventing and/or inhibiting the proliferation of the flatworm.

Additionally or alternatively the pathogen or pest can also be a plant pathogen or pest, such as
a) an oomycete selected from the genus Pythium causing the disease damping off;
b) an oomycete selected from the genus Phytophthora causing the disease potato late blight/Phytophthora blight/root & stem rot/downy mildew/black shank; and/or
c) a fungus selected from i) the genus Fusarium causing the disease Fusarium wilt/Panama disease, ii) the genus Verticillium causing the disease verticillium wilt

Additionally or alternatively the pathogen or pest can also be a human pathogen or pest such as a sporozoan selected from the genus Cryptosporidium contaminating water causing the disease cryptosporidiosis in humans and wherein the pathogen or pest is contacted with the lipopeptide biosurfactant as a disinfectant in an amount effective of preventing and/or inhibiting the excystation of the oocysts.

Additionally or alternatively the pathogen or pest can also be an avian pathogen or pest such as a sporozoan selected from the genus Eimeria causing the disease coccidiosis in avians and wherein the pathogen or pest is contacted with the lipopeptide biosurfactant as a disinfectant in an amount effective of preventing and/or inhibiting the sporulation of the oocysts.

The effective amount of the lipopeptide biosurfactant is suitably a concentration in the aqueous solution of from 5 μg/mL to 1000 mg/L. In some embodiments, in particular in the treatment of micro-algae causing red/green tide, the concentration of the lipopeptide biosurfactant in the aqueous solution is preferably between 0.1 to 1000 mg/L, optionally 0.5 to 500 mg/L, optionally 1 to 100 mg/L, optionally 2 to 50 mg/l, optionally 5 to 25 mg/L.

Also described herein are lipopeptide biosurfactants effective in treating infections in a subject resulting by one or more pathogens or pests selected from Ciliates, which is not Ichthyophthirius multifiliis; Flagellated protists, including dinoflagellates; Flatworms; Amoebae; Bacteria, including cyanobacteria; Viruses; Oomycetes; and/or Fungi.

In some embodiments the subject to be treated is a fish. The fish may be a hypersaline, a marine, a brackish or a freshwater fish, preferably useful for farming for consumption or as an ornamental fish.

For freshwater fish, the lipopeptide surfactant of the invention has been found to be effective in the treatment of
a) trichodiniasis, where the pathogen or pest is a ciliate parasite of the genus Trichodina;
b) chilodonellosis, where the pathogen or pest is a ciliate parasite of the genus Chilodonella;
c) guppy disease, where the pathogen or pest is a ciliate parasite of the genus Tetrahymena;
d) ichthyobodiasis (costiasis), where the pathogen or pest is a flagellated protist of the genus Ichthyobodo (Costia);
e) gyrodactylosis, where the pathogen or pest is a flatworm of the genus Gyrodactylus;
f) dactylogyrosis, where the pathogen or pest is a flatworm of the genus Dactylogyrus;
g) furunculosis or tail rot or fin rot or enteritis or hemorrhagic septicemia, where the pathogen or pest is a bacterium of the genus Aeromonas;
h) bacterial gill disease or rainbow trout fry syndrome or columnaris, where the pathogen or pest is a bacterium of the genus Flavobacterium;
i) streptococcosis, where the pathogen or pest is a bacterium selected from the genus Streptococcus; and/or
j) saprolegniasis, where the pathogen or pest is an oomycete selected from the genus Saprolegnia or Achlya.
k) epizootic ulcerative syndrome (EUS) or crayfish plague, where the pathogen or pest is an oomycete selected from the genus Aphanomyces.

For freshwater fish, the lipopeptide surfactant is in some embodiments effective in the treatment of trichodiniasis, where the pathogen or pest is a ciliate parasite of the genus Trichodina;
a) chilodonellosis, where the pathogen or pest is a ciliate parasite of the genus Chilodonella;
b) guppy disease, where the pathogen or pest is a ciliate parasite of the genus Tetrahymena;
c) ichthyobodiasis (costiasis), where the pathogen or pest is a flagellated protist of the genus Ichthyobodo (Costia);
d) furunculosis or tail rot or fin rot or enteritis or hemorrhagic septicemia, where the pathogen or pest is a bacterium of the genus Aeromonas;
e) bacterial gill disease or rainbow trout fry syndrome or columnaris, where the pathogen or pest is a bacterium of the genus Flavobacterium;
f) streptococcosis, where the pathogen or pest is a bacterium selected from the genus Streptococcus; and/or
g) saprolegniasis, where the pathogen or pest is an oomycete selected from the genus Saprolegnia.
h) epizootic ulcerative syndrome (EUS) or crayfish plague, where the pathogen or pest is an oomycete selected from the genus Aphanomyces.

For marine fish, the lipopeptide surfactant of the invention has been found to be effective in the treatment of
a) marine velvet, where the pathogen or pest is a dinoflagellate parasite of the genus Amyloodinium;
b) sparicotylosis, where the pathogen or pest is a flatworm of the genus Sparicotyle;
c) amoebic gill disease, where the pathogen or pest is an amoeba of the genus Neoparamoeba;
d) pseudotuberculosis or fish pasteurellosis, where the pathogen or pest is a bacterium of the genus Photobacterium;
e) vibriosis, where the pathogen or pest is a bacterium of the genus Vibrio; and/or
f) viral hemorrhagic septicemia (VHS) or infectious hematopoietic necrosis (IHN) where the pathogen or pest is a virus of genus Novirhabdovirus.

For marine fish, the lipopeptide surfactant is in some embodiments effective in the treatment of
a) pseudotuberculosis or fish pasteurellosis, where the pathogen or pest is a bacterium of the genus Photobacterium;
b) vibriosis, where the pathogen or pest is a bacterium of the genus Vibrio; and/or
c) viral hemorrhagic septicemia (VHS) where the pathogen or pest is a virus of genus Novirhabdovirus.

In some embodiments the subject to be treated is a mollusk. The mollusk may be a marine or a freshwater mollusk useful for farming and consumption. For mollusks the lipopeptide surfactant of the invention has been found to be effective in the treatment of
a) dermo disease, where the pathogen or pest is a dinoflagellate parasite of the genus Perkinsus;
b) pacific oyster nocardiosis, where the pathogen or pest is a bacterium of the genus Nocardia; and/or
c) abalone tubercle mycosis, where the pathogen or pest is an oomycete selected from the genus Halioticida.

In some embodiments the subject to be treated is a crustacean. The crustacean may be a marine or a freshwater crustacean preferably useful for farming and consumption. For crustaceans such as shrimps, in particular whiteleg shrimps, the lipopeptide surfactant of the invention has been found to be effective in the treatment of white spot syndrome, where the pathogen or pest is a virus of the genus Whispovirus.

In some embodiments the subject to be treated is a cultivated plant. For plants the lipopeptide surfactant of the invention has been found to be effective in the treatment of
a) citrus greening disease in citrus trees, where the pathogen or pest is a bacterium of the genus Candidatus;
b) damping off in crop plants, where the pathogen or pest is an oomycete of the genus Pythium;
c) potato late blight or Phytophthora blight or root & stem rot or downy mildew or black shank in crop plants, where the pathogen or pest is an oomycete of the genus Phytophthora;
d) Fusarium wilt or Panama disease in crop plants, where the pathogen or pest is a fungus of the genus Fusarium;
e) Verticillium wilt, where the pathogen or pest is a fungus of the genus Verticillium;
f) root rot in crop plants, where the pathogen or pest is a fungus of the genus Rhizoctonia; and/or
g) gray mold in plants, where the pathogen or pest is a fungus of the genus Botrytis.

In some embodiments the subject to be treated is an animal. For animals, including humans, the lipopeptide surfactant of the invention has been found to be effective in the treatment of schistosomiasis, where the pathogen or pest is a flatworm of the genus Schistosoma, and wherein the pathogen or pest is contacted with a lipopeptide biosurfactant in an amount effective of killing and/or preventing and/or inhibiting the proliferation of the flatworm.

The lipopeptide biosurfactant useful for the treatment of diseases in animals or plants are described supra.

The lipopeptide surfactant of the invention is applied to the subject in a suitable manner enabling effective treatment of a subject. Where the subject to be treated is aquacultured species, fish, mollusks etc, the lipopeptide biosurfactant of the invention can be added to the water where the subject is kept and cultured. Alternatively, the lipopeptide biosurfactant can also be produced in situ by adding and cultivating a microbial source producing the lipopeptide biosurfactant to the water where the subject is kept and cultured. The lipopeptide biosurfactant can also be administered by spiking the lipopeptide biosurfactant of the invention into a feed for the subject which is then ingested by the subject. Concentrations of the lipopeptide biosurfactant in the water should be kept between 5 to 1000 μg/ml, such as 10 to 100 μg/ml of the lipopeptide biosurfactant. In some embodiments suitable concentrations can range from 30 to 70 μg/ml, especially about 50 μg/ml. In other embodiments further embodiments suitable concentrations can be at least 10 μg/ml, especially at least 30 μg/ml, such as at least 50 μg/ml. In still further embodiments suitable concentrations can be up to 500 μg/ml, especially up to 200 μg/ml, such as up to 100 μg/ml.

Where the subject is a plant the lipopeptide biosurfactant can be applied administering a powder or a solution comprising the lipopeptide biosurfactant of the invention to the plant, or alternatively administering a microbial source producing the lipopeptide biosurfactant in situ on the plant to be treated.

Where the subject is an animal the lipopeptide biosurfactant of the invention can be administered enterally, parenterally or topically.

Suitable administration of the lipopeptide surfactant of the invention includes one-time administration or repeated administrations and can also include bolus administrations to achieve peak concentrations, optionally supplemented with lower concentration maintenance administrations.

In some aspects, the a lipopeptide biosurfactant as defined herein is used in a closed or semi-closed water flow system that comprises a bacterial water filter, whereby the lipopeptide biosurfactant works on a target pathogen but the bacterial water filter is unharmed.

### Items

1. A method for the killing, inactivating, or inhibiting of one or more pathogens or pests selected from
a) Ciliates, which is not Ichthyophthirius multifiliis or Cryptocaryon irritans;
b) flagellated protists, including dinoflagellates;
c) Flatworms;
d) Amoebae;
e) Bacteria, Including cyanobacteria;
f) Algae;
g) Viruses;
h) Oomycetes, which is not Saprolegnia diclina; and/or
i) Fungi,
   comprising contacting the pathogen or pest with an effective amount of a lipopeptide biosurfactant.
   2. The method of any preceding item, wherein the lipopeptide biosurfactant comprises
a) a viscosin or viscosin-like lipopeptide or a derivative thereof;
b) a massetolide or a derivative thereof and/or
c) a putisolvin or a derivative thereof
   or any combination thereof
   3. The method of any preceding item, wherein the lipopeptide biosurfactant is isolated from a microbial source, optionally from a bacterium, a fungus or an algae.
   4. The method of item 3, wherein the microbial source is a bacterium, optionally of the genus Pseudomonas fluorescens, optionally Pseudomonas fluorescens strain H6 or the Pseudomonas fluorescens strain SDW1 deposited under deposit number DSMZ-34058.
   5. The method of item 1 to 4, wherein the lipopeptide biosurfactant is a viscosin or viscosin-like lipopeptide or a derivative thereof.
   6. The method of item 5, comprising wherein a viscosin-like lipopeptide or a derivative thereof isolated from Pseudomonas fluorescens strain H6 or DSMZ-34058.
   7. The method of any preceding item, wherein the pathogen or pest is contacted with the lipopeptide biosurfactant in an aqueous solution comprising an effective amount of the lipopeptide biosurfactant to kill, inactivate, or inhibit the pathogen or pest.
   8. The method of item 7, wherein the aqueous solution is a marine solution or a freshwater solution or a brackish solution or a hypersaline solution.
   9. The method of item 7 to 8, wherein the pathogen or pest is a cyanobacterium or an algae.
   10. The method of item 9, wherein the cyanobacterium or algae is a harmful cyanobacterium or algae capable of causing Harmful Algal Bloom (HAB) in marine, brackish or freshwater environments.
   11. The method of item to 10, wherein the algae is selected from the class Dinophyceae, Pelagophyceae, Raphidophyceae, or Prymnesiophyceae or a combination thereof.
   12. The method of item 11, wherein the Dinophyceae is of the order Actiniscales, Amphidiniales, Amphilothales, Blastodiniales, Brachidiniales, Coccidiniales, Dinophysiales, Gloeodiniales, Gonyaulacales, Gymnodiniales, Lophodiniales, Noctilucales, Oxyrrhinales, Peridiniales, Phytodiniales, Prorocentrales, Pyrocystales, Suessiales, Syndiniales, Thoracosphaerales, Torodiniales or Tovelliales or a combination thereof.
   13. The method of item 12, wherein the
a) Gonyaulacales is of i) the family Ostreopsidaceae, optionally of the genus Alexandrium, optionally of the species A. tamarense; optionally of the genus Gambierdiscus, optionally the species G. toxicus, or ii) the family Ceratiaceae, optionally the genus Ceratium;
b) Noctilucales is of the family Noctilucaceae, optionally of the genus Noctiluca; and/or
c) Gymnodiniales is of the family i) Kareniaceae, optionally the genus Karenia; optionally of the species K. brevis; or ii) Gymnodiniaceae, optionally the genus Cochlodinium.
   14. The method of item 11, wherein the Pelagophyceae is of the order Pelagomonadales or Sarcinochrysidales or a combination thereof.
   15. The method of item 14, wherein the Pelagomonadales is of the genus Aureococcus, optionally the species A. anophagefferens.
   16. The method of item 11, wherein the Raphidophyceae is of the order Actinophryida, Chattonellales, Commatiida, or Raphidomonadales or a combination thereof.
   17. The method of item 16, wherein the Chattonellales is of the genus Heterosigma, optionally the species H. akashiwo.
   18. The method of item 11, wherein the Prymnesiophyceae is of the order Coccolithales, Coccosphaerales, Isochrysidales, Phaeocystales, Prymnesiales, Prymnesiophyceae incertae sedis, Syracosphaerales, or Zygodiscales.
   19. The method of item 18, wherein the Prymnesiales is of the genus Prymnesium, optionally the species P. parvum.
   20. The method of item 10, wherein the cyanobacterium is of the order Chroococcales, optionally of the family Microcystaceae, optionally of the genus Microcystis.
   21. The method of item 1 to 8, wherein the aqueous solution is freshwater and wherein the pathogen or pest is
a) a ciliate parasite selected from the genus Trichodina causing the disease trichodiniasis;
b) a ciliate parasite selected from the genus Chilodonella causing the disease chilodonellosis;
c) a ciliate parasite selected from the genus Tetrahymena causing the disease guppy disease;
d) a flagellated protists selected from the genus Ichthyobodo (Costia) causing the disease ichthyobodiasis (costiasis);
e) a flatworm selected from the genus Gyrodactylus causing the disease gyrodactylosis;
f) a flatworm selected from the genus Dactylogyrus causing the disease dactylogyrosis; and/or
g) an oomycete selected from i) the genus Achlya causing the disease saprolegniasis and/or ii) the genus Aphanomyces causing the disease epizootic ulcerative syndrome (EUS)/crayfish plague.
   22. The method of item 1 to 8, wherein the aqueous solution is marine and wherein the pathogen or pest is
a) a dinoflagellate parasite selected from the genus Amyloodinium causing the disease marine velvet;
b) a flatworm selected from the genus Sparicotyle causing the disease sparicotylosis;
c) an amoebae selected from the genus Neoparamoeba causing the disease amoebic gill disease; and/or
e) a virus selected from the genus Novirhabdovirus causing the disease viral hemorrhagic septicemia (VHS)/infectious hematopoietic necrosis (IHN).
   23. The method of item 1 to 8, wherein the pathogen or pest is:
a) a dinoflagellate parasite selected from the genus Perkinsus causing the disease dermo disease in mollusks;
b) a bacterium selected from the genus Nocardia causing the disease pacific oyster nocardiosis in mollusks; and/or
c) an oomycete selected from the genus Halioticida causing the disease abalone tubercle mycosis in mollusks.
   24. The method of item 1 to 8, wherein the pathogen or pest is a virus selected from the genus Whispovirus causing the disease white spot syndrome in whiteleg shrimp
   25. The method of item 1 to 6, wherein the pathogen or pest is:
a) a bacterium selected from the genus Candidatus causing the disease citrus greening disease in plants;
b) an oomycete selected from the genus Pythium causing the disease damping off in plants;
c) an oomycete selected from the genus Phytophthora causing the disease potato late blight/Phytophthora blight/root & stem rot/downy mildew/black shank in plants; and/or
d) a fungus selected from i) the genus Fusarium causing the disease Fusarium wilt/Panama disease in plants, ii) the genus Verticillium causing the disease verticillium wilt in plants, iii) the genus Rhizoctonia causing the disease root rot in plants, and/or iv) the genus Botrytis causing the disease gray mold in plants.
   26. The method of item 1 to 8, wherein the pathogen or pest is a flatworm selected from the genus Schistosoma causing the disease schistosomiasis in humans and wherein the pathogen or pest is contacted with a lipopeptide biosurfactant in an amount effective of preventing and/or inhibiting the proliferation of the flatworm.
   27. The method of item 7 to 24 any preceding item, wherein concentration of lipopeptide biosurfactant in the aqueous solution is from 5 μg/mL to 1000 mg/L.
   28. The method of item 27, wherein the effective amount of the lipopeptide biosurfactant is between 0.1 to 1000 mg/L, optionally 0.5 to 500 mg/L, optionally 1 to 100 mg/L, optionally 2 to 50 mg/l, optionally 5 to 25 mg/L.
   29. A lipopeptide biosurfactant for use in the treatment of an infection in a subject by one or more pathogens or pests selected from
a) Ciliates, which is not Ichthyophthirius multifiliis' or Cryptocaryon irritans;
b) flagellated protists, including dinoflagellates;
c) Flatworms;
d) Amoebae;
e) e) Bacteria, including cyanobacteria;
f) Viruses;
g) Oomycetes, which is not Saprolegnia diclina; and/or
h) Fungi.
   30. The lipopeptide biosurfactant of item 29, wherein the subject is a freshwater fish (including ornamental fish and aquaculture fish) and wherein
a) the pathogen or pest is a ciliate parasite selected from the genus Trichodina, and the disease is trichodiniasis;
b) the pathogen or pest is a ciliate parasite selected from the genus Chilodonella, and the disease is chilodonellosis;
c) the pathogen or pest is a ciliate parasite selected from the genus Tetrahymena, and the disease is guppy disease;
d) the pathogen or pest is a flagellated protist selected from the genus Ichthyobodo (Costia), and the disease is ichthyobodiasis (costiasis);
e) the pathogen or pest is a flatworm selected from the genus Gyrodactylus, and the disease is gyrodactylosis;
f) the pathogen or pest is a flatworm selected from the genus Dactylogyrus, and the disease is dactylogyrosis;
g) the pathogen or pest is a bacterium selected from the genus Aeromonas, and the disease is furunculosis or tail rot or fin rot or enteritis or hemorrhagic septicemia;
h) the pathogen or pest is a bacterium selected from the genus Flavobacterium, and the disease is bacterial gill disease/rainbow trout fry syndrome/columnaris;
i) the pathogen or pest is a bacterium selected from the genus Streptococcus, and the disease is streptococcosis; and/or
j) the pathogen or pest is an oomycete selected from i) the genus Achlya, and the disease is saprolegniasis and/or ii) the genus Aphanomyces, and the disease is epizootic ulcerative syndrome (EUS)/crayfish plague.
   31. The lipopeptide biosurfactant of item 29, wherein the subject is a marine fish (including ornamental fish and aquaculture fish) and wherein
a) the pathogen or pest is a dinoflagellate parasite selected from the genus Amyloodinium, and the disease is marine velvet;
b) the pathogen or pest is a flatworm selected from the genus Sparicotyle, and the disease is sparicotylosis;
c) the pathogen or pest is an amoebae selected from the genus Neoparamoeba, and the disease is amoebic gill disease;
d) the pathogen or pest is a bacterium selected from the genus Photobacterium, and the disease is pseudotuberculosis/fish pasteurellosis;
e) the pathogen or pest is a bacterium selected from the genus Vibrio, and the disease is vibriosis; and/or
f) the pathogen or pest is a virus selected from the genus Novirhabdovirus, and the disease is viral hemorrhagic septicemia (VHS)/infectious hematopoietic necrosis (IHN).
   32. The lipopeptide biosurfactant of item 28, wherein the subject is a mollusk and wherein
a) the pathogen or pest is a dinoflagellate parasite selected from the genus Perkinsus, and the disease is dermo disease;
b) the pathogen or pest is a bacterium selected from the genus Nocardia, and the disease is pacific oyster nocardiosis; and/or
c) the pathogen or pest is an oomycete selected from the genus Halioticida, and the disease is abalone tubercle mycosis.
   33. The lipopeptide biosurfactant of item 29, wherein the subject is a whiteleg shrimp and wherein the pathogen or pest is a virus selected from the genus Whispovirus, and the disease is white spot syndrome.
   34. The lipopeptide biosurfactant of item 29, wherein the subject is human and wherein the pathogen or pest is a flatworm selected from the genus Schistosoma, and the disease is schistosomiasis and wherein the pathogen or pest is contacted with a lipopeptide biosurfactant in an amount effective of preventing and/or inhibiting the proliferation of the flatworm.
   35. The lipopeptide biosurfactant of any preceding item, wherein the lipopeptide biosurfactant comprises
a) a viscosin or viscosin-like lipopeptide or a derivative thereof;
b) a massetolide or a derivative thereof and/or
c) a putisolvin or a derivative thereof or any combination thereof
   36. The lipopeptide biosurfactant of any preceding item, wherein the lipopeptide biosurfactant is isolated from a microbial source, optionally from a bacterium, a fungus or an algae.
   37. The lipopeptide biosurfactant of item 36, wherein the microbial source is a bacterium, optionally of the genus Pseudomonas fluorescens, optionally Pseudomonas fluorescens strain H6 or the Pseudomonas fluorescens strain SDW1 deposited under deposit number DSMZ-34058.
   38. The lipopeptide biosurfactant item 37, wherein the lipopeptide biosurfactant is a viscosin or viscosin-like lipopeptide or a derivative thereof.
   39. The lipopeptide biosurfactant of item 38, comprising wherein a viscosin-like lipopeptide or a derivative thereof isolated from Pseudomonas fluorescens strain H6 or DSMZ-34058.
   40. The lipopeptide biosurfactant or the method of any preceding item, wherein the pathogen or pest is contacted with a composition comprising from 5-1000 μg/ml of the lipopeptide biosurfactant.
   41. A bacterial isolate for use in the killing, inactivating, or inhibiting of one or more pathogens or pests selected from
a) Ciliates, which is not Ichthyophthirius multifiliis or Cryptocaryon irritans;
b) Flagellates, including dinoflagellates;
c) Flatworms;
d) Amoebae;
e) Bacteria, including cyanobacteria;
f) Viruses;
g) Oomycetes, which is not Saprolegnia diclina; and/or
h) Fungi;
   wherein the bacterial isolate comprises bacteria that produce a lipopeptide surfactant.
   42. The bacterial isolate of item 41 wherein the bacteria produce a massetolide or derivative thereof.
   43. The bacterial isolate of item 41 to 42, wherein the bacteria produce a putisolvin or a derivative thereof.
   44. The bacterial isolate of item 41 to 43, wherein the bacteria comprise Pseudomonas sp. strain H6 or or DSMZ-34058.
   45. Use of a lipopeptide biosurfactant as defined in any one of the preceding items in a closed or semi-closed water flow system that comprises a bacterial water filter, whereby the lipopeptide biosurfactant works on a target pathogen but the bacterial water filter is unharmed.

### EXAMPLES

### Materials and Methods

### Materials

Chemicals used in the examples herein e.g. for buffers and substrates are commercial products of at least reagent grade. (NH₄)₂SO₄, Na₂HPO₄, KH₂PO₄, NaCl, HCl, NaOH, NH₃·H₂O and corn steep liquor are used in the fermentation medium and purification reagent.

### Analytical Procedures

Detection and quantification of lipopeptide surfactant (LS) was performed on Agilent Technologies 1200 series High Performance Liquid Chromatogram (HPLC) equipped with Luna® 5 μm C18(2) 100 Å, LC Column 150×2 mm (Part No. #00F-4252-BO, Phenomenex). Mobile phases of water with 0.1% (v/v) trifluoroacetic acid (TFA) (Solution A) and acetonitrile (ACN) with 0.085% (v/v) TFA (Solution B) were used. The absorbance at 210 nm was used to detect LS. One to five microliters of sample were injected onto the column. The flow rate was set at 0.9 ml/min. The HPLC running program was started with 90% of the solution A and 10% of the solution B, after running 2 minutes, the solution B was increased to 80% with gradient and the solution A reduced to 20%. After 8 minutes, the solution B was increased to 100% and kept running for 1 minute, between the 9 minutes to 10 minutes, the solution B was reduced back from 100% to 10% and the solution A was increased from 0% to 90%. LS purified by AnalytiCon Discovery GmbH (Germany) using preparative HPLC was used as standard LS with >99.5% purity for quantification.

### Preparation of Fish Subjects for In Vivo Testing

In all in vivo efficacy tests, fish were acclimatized, regarding the light/dark cycle and temperature, to the experimental conditions for 14 days prior to performing the experiments.

### Example 1—Culturing Pseudomonas sp. Producing Lipopeptide Biosurfactant

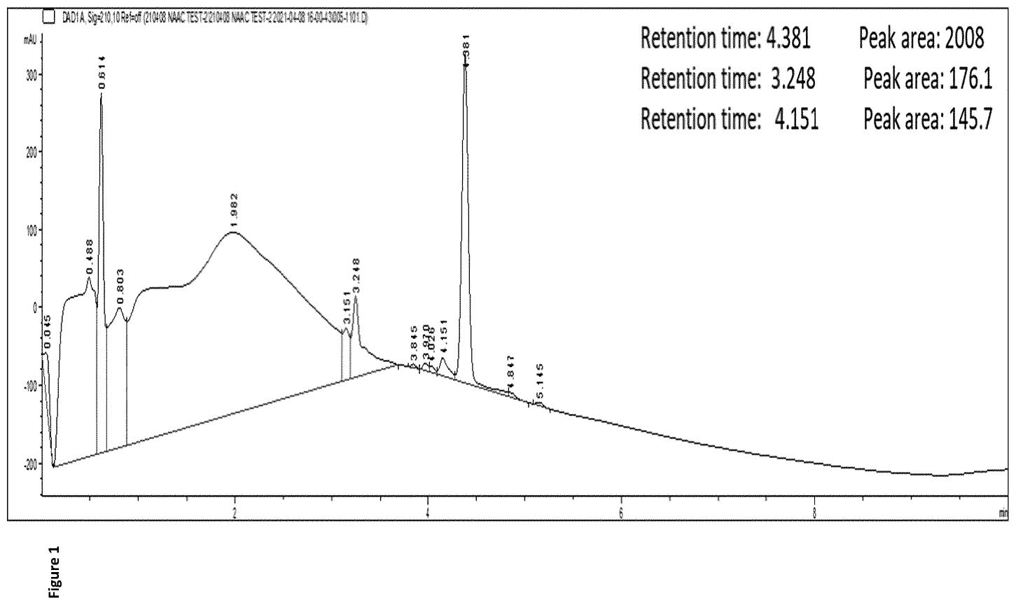

A strain of Pseudomonas sp., strain LMG 5329 available from Belgiun Coordinated Collections of Microorganisms (BCCM)—see: https://bccm.belspo.be/catalogues/lmg-strain-details?NUM=5329&COLTYPE=&LIST1=STRNUM&TEXT1=5329&LIST2=SPECIES&TEXT2=&LIST3=ORIG SUBST&TEXT3=&LIST4=ORIGIN&TEXT4=&LIST5=ALL%20FIELDS&TEXT5=&CONJ=OR&RANGE=20 was fermentated and lipopeptide biosurfactant was isolated and purified using the methodology of Liu et al 2015 and/or in WO2019101739. This strain is also known from Rokni-Zadeh et al 2013 and Oni et al 2020 to produce the lipopeptide White Line Inducing Factor (WLIP). HPLC analysis of the resulting isolate revealed a major product peak of lipopeptide (FIG. 1).

A stock solution of the lipopeptide surfactant (LS) was prepared prior to the tests by dissolving the freeze-dried LS in distilled water and preparing a dilution series ranging from 5 to 500 μg/ml for pathogen or pest exposures. Negative control (pathogen or pest and distilled water), blank control (distilled water and media), and turbidity control (LS and media) were included in all tests.

### Example 2—Culturing Deposited Pseudomonas fluorescens H6 Producing Lipopeptide Biosurfactant

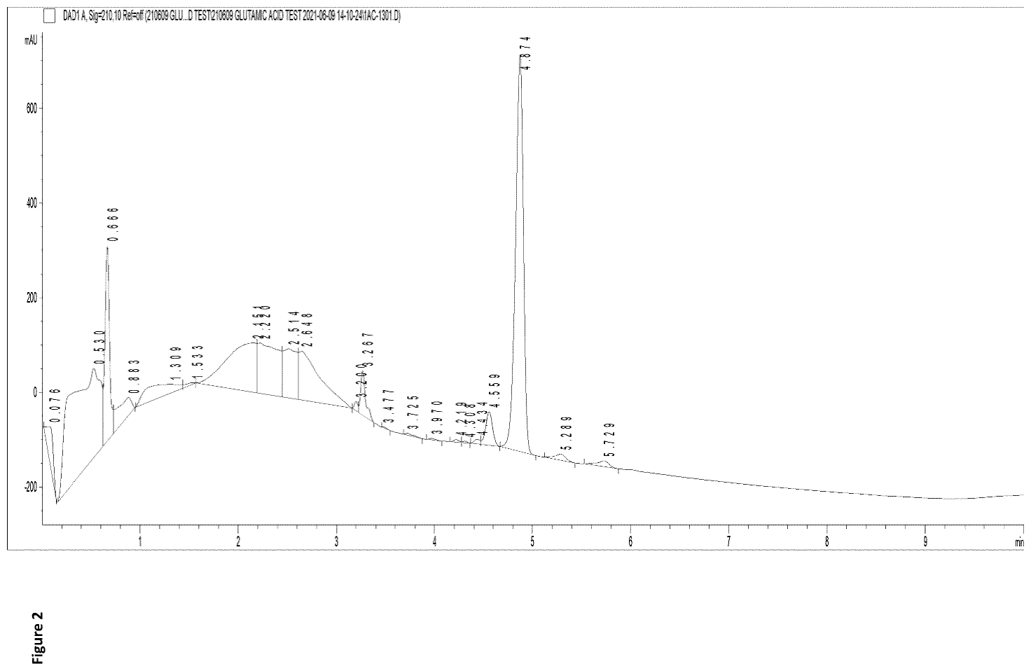

Lipopeptide biosurfactant of Pseudomonas fluorescens strain H6 was extracted according to the method described by Liu et al. (2015) and/or WO2019/101739. The Pseudomonas fluorescens strain H6 deposited under CBS 143505 was grown on Pseudomonas agar plates for 48 hours at 25° C. Cells of strain H6 were collected from the agar plates. Cells were collected from the agar plates and suspended in sterile de-mineralized water and mixed to homogenize the cell suspension. Cell suspensions were then centrifuged twice for 10 min at 9,000 rpm at 4° C. and the supernatant was filter-sterilised with 0.2 um filters. The lipopeptide biosurfactant present in the cell-free culture supernatant was precipitated by acidification of the supernatant with 9% (v/v) HCl to pH 2.0. Precipitation was allowed for 1 hour on ice. The precipitate was collected by centrifugation at 12000 g, 4° C. for 15 min speed and washed with acidified (pH 2.0) demineralized water. Demineralized water was added to the washed precipitate and the pH was adjusted to 8.0 with 0.2 M NaOH to allow the precipitate to dissolve. The resulting solution of lipopeptide biosurfactant was freeze-dried in a vacuum freeze dryer. A HPLC analysis chromatogram is shown in FIG. 2, showing a retention time of the lipopeptide surfactant of 4.874 minutes.

A stock solution of the lipopeptide surfactant (LS) was prepared prior to the tests by dissolving the freeze-dried LS in distilled water and preparing a dilution series ranging from 5 to 500 μg/ml for pathogen or pest exposures. Negative control (pathogen or pest and distilled water), blank control (distilled water and media), and turbidity control (LS and media) were included in all tests.

### Example 3—Culturing Deposited Pseudomonas fluorescens SDW1 (DSMZ-34058) Producing Lipopeptide Biosurfactant

Lipopeptide biosurfactant of Pseudomonas fluorescens strain SDW1 was extracted according to the method described by Liu et al. (2015) and/or WO2019/101739. The Pseudomonas fluorescens strain SDW1 was grown on Pseudomonas agar plates for 48 hours at 25° C. Cells of strain SDW1 were collected from the agar plates. Cells were collected from the agar plates and suspended in sterile de-mineralized water and mixed to homogenize the cell suspension. Cell suspensions were then centrifuged twice for 10 min at 9,000 rpm at 4° C. and the supernatant was filter-sterilised with 0.2 um filters. The lipopeptide biosurfactant present in the cell-free culture supernatant was precipitated by acidification of the supernatant with 9% (v/v) HCl to pH 2.0. Precipitation was allowed for 1 hour on ice. The precipitate was collected by centrifugation at 12000 g, 4° C. for 15 min speed and washed with acidified (pH 2.0) demineralized water. Demineralized water was added to the washed precipitate and the pH was adjusted to 8.0 with 0.2 M NaOH to allow the precipitate to dissolve. The resulting solution of lipopeptide biosurfactant was freeze-dried in a vacuum freeze dryer.

A stock solution of the lipopeptide surfactant (LS) was prepared prior to the tests by dissolving the freeze-dried LS in distilled water and preparing a dilution series ranging from 5 to 500 μg/ml for pathogen or pest exposures. Negative control (pathogen or pest and distilled water), blank control (distilled water and media), and turbidity control (LS and media) were included in all tests.

### Example 4—In Vitro Testing of the Lipopeptide Surfactant Effect on Ciliates

Ciliates are propagated in a T-25 flask containing a suitable culture medium and harvested 1-2 days after reaching peak density (quantified by hemacytometer). Volumes of 100 μl of diluted ciliate culture are aliquoted into tubes containing 100 μl of LS from example 1, 2 and 3 at concentrations ranging from 5 to 160 μg/ml (total volume 200 μl), vortexed, and transferred into 12-well cell culture plates together with controls (each sample in quadruplicate). Plates are covered, incubated at room temperature (22-25° C.), and observed at 0, 15, 30, 45, 60, 90, and 120 minutes for motility and MIC confirmation under a stereomicroscope based on observations of ciliate motility. Non-motile and lysed ciliates are considered dead.

### Results

LS concentrations between 10 and 30 μg/ml and above are lethal for tested ciliates within 30-60 min of exposure, whereas concentrations lower than 10 μg/ml shows little or no effect on the pathogens or pests.

### Example 5—In Vitro Testing of the Lipopeptide Surfactant Effect on Flagellated Protists/Dinoflagellates

Flagellated protists/dinoflagellates are propagated in a T-25 flask containing a suitable culture medium and harvested 1-2 days after reaching peak density (quantified by hemacytometer). Volumes of 100 μl of diluted flagellated protist culture are aliquoted into tubes containing 100 μl of LS from example 1, 2 and 3 at concentrations ranging from 5 to 160 μg/ml (total volume 200 μl), vortexed, and transferred into 12-well cell culture plates together with controls (each sample in quadruplicate). Plates are covered, incubated at room temperature (22-25° C.), and observed at 0, 15, 30, 45, 60, 90, and 120 minutes for motility and MIC confirmation under a dissecting microscope based on observations of flagellated protist motility. Non-motile flagella and lysed cells are considered dead.

### Results

LS concentrations between 10 and 30 μg/ml and above are lethal for tested flagellated protists/dinoflagellates within 30-60 min of exposure, whereas concentrations lower than 10 μg/ml shows no effect on the pathogens or pests.

### Example 6—In Vitro Testing of the Lipopeptide Surfactant Effect on Flatworms

To provide the source of flatworms (monogeneans) for tests, a laboratory infection is established under controlled conditions. To obtain parasites, fish from laboratory infection are anaesthetized and live parasites are removed from the fish surface and transferred into 12-well cell culture plates. Volumes of 200 μl of LS from example 1, 2 and 3 at concentrations ranging from 5 to 160 μg/ml are added into the plate wells together with controls (each sample in quadruplicate). Plates are incubated at room temperature and observed for survival and egg production under a stereomicroscope after 1 h, 2 h, 3 h, 4 h, 8 h, 12 h, and 24 h of exposure. Parasite showing no signs of motion and failing to respond to tactile stimulus are considered dead (Trasviña-Moreno et al. 2017).

### Results

LS concentrations between 10 and 30 μg/ml and above are lethal for tested flatworm within 30-60 min of exposure, whereas concentrations lower than 10 μg/ml shows no effect on the pathogens or pests.

### Example 7—In Vitro Testing of the Lipopeptide Surfactant Effect on Amoebae

Isolated amoebae from infected fish are cultured on malt yeast agar (0.01% malt, 0.01% yeast, 2% Bacto agar, 0.2 am filtered sea water with 35% salinity) overlaid with 0.2 am filtered sea water. Plates are incubated at 18° C. (Cano et al. 2019). Amoebae are subcultured by transferring amoebae by pipetting from the agar plate to wells in a 24-well cell culture plate containing malt yeast agar (1 ml in each well) and moistened with Neff's amoeba saline (Jensen et al. 2020). Subcultures are allowed to establish over weeks and cell counting is performed in a haemocytometer. When dense amoeba layers (more than 20 live amoebae in a 20 μl) is achieved, volumes of 500 μl of LS from example 1, 2 and 3 at concentrations ranging from 5 to 500 μg/ml are added into the plate wells together with controls (each sample in quadruplicate). Plates are incubated for 24 h, and viability of amoeba are recorded at 5 min, 30 min, 1 h, 2 h, 24 h. Amoebae with shrunken appearance, lack of movements, and no cytoplasmic activity are considered dead (Jensen et al. 2020).

### Results

After 24 h, all amoebae exposed to 250 μg/ml and above are dead. Both live and dead amoebae are observed in wells exposed to 125 μg/ml.

### Example 8—Testing of the Lipopeptide Surfactant Effect on Nitrifying Bacteria

Lipopeptide biosurfactant of DSMZ 34058 (hereinafter referred to as LP34058) obtained in example 3 was tested for impacts on nitrifying bacteria in biofilters.

Biofilter bacteria in fish tanks have the key function of nitrifying the ammonia derived from fish feces and leftover food and converting it into the less toxic nitrite and subsequently nitrate.

### Methods:

To evaluate the effect of LP34058 on nitrifying bacteria, biofilters from an earlier fish challenge study treated with low dose (6.3 mg active compound/L) and high dose (12.6 mg active compound/L) of LP34058 were used for a new study targeting the water quality. In this study the total ammonia nitrogen (TAN), nitrite and nitrate were measured for 15 consecutive days using test strips.

The study consisted of 6 arms: A) No fish, no biofilter; B) No fish+biofilter not exposed to LP34058; C) Six fish+biofilter previously exposed to “low dose” LP34058; D) No fish+biofilter, previously exposed to “low dose” LP34058”); E) Six fish+biofilter, previously exposed to “high dose” LP34058; and F) No fish+biofilter, previously exposed to “high dose” LP34058. Feed was added every second day, and no water exchange was performed during the study.

### Results:

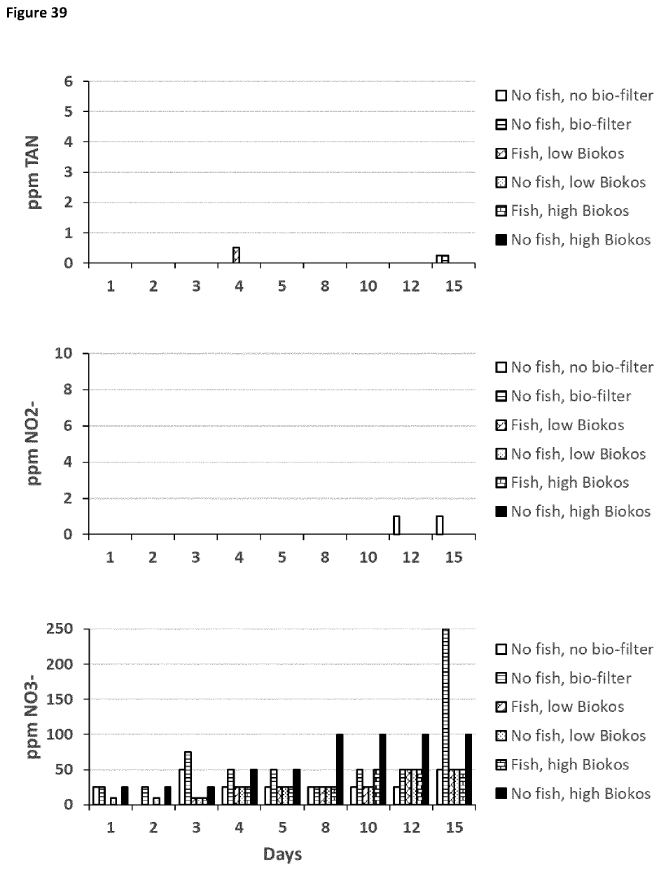

No fish from arms C and E showed any signs of intoxication, indicating good water quality and full functionality of biofilter. These observations were in accordance with the concentrations of TAN, nitrite and nitrate measured (FIG. 39). The sensitivity level for the strip for the TAN assay is 6 ppm, and levels of 0.25 ppm and 0.5 ppm were recorded at two and one time point, respectively. These values are considered safe for fish at the pH levels measured (between 6.8 and 7.8). For nitrite, a value above the sensitivity of the assay, namely 1 ppm, was measured only in 2 cases, both in group A with neither fish nor biofilter. Finally, for nitrate, values up to 100 ppm (group F) and an outlier at 250 ppm (group B) were detected. For all groups containing fish in the tank, values did not exceed 50 ppm. This value is at the end of the margin of safety for fish, but the absence of growing plants in the tanks and water change should also be considered.

### Conclusion:

The study confirms that LP34058 does not affect biofilter performance.

### Example 9—In Vitro Testing of the Lipopeptide Surfactant Effect on Oomycetes/Fungi

Oomycete/fungus isolates are grown on agar dish containing glucose and yeast extract until covering the dish. A small section of agar is cut out and inverted onto a polycarbonate filter membrane and incubated until mycelium reaches the edge of the membrane. Mycelia and filters are transferred in quadruplicate to sterile petri dishes containing different concentrations of LS from example 1 and 2 ranging from 10 to 320 μg/ml. For each concentration, a control dish containing distilled water only (no LS) and a control dish containing no oomycete is included. Membranes are kept in LS solution for 1 day, and then washed with distilled water and transferred to an agar plate. Agar plates are then incubated at respective conditions regarding temperature and incubation time. Following incubation, hyphal growth is measured, and morphological abnormalities are evaluated under stereomicroscope.

### Results

Exposure to LS show almost complete growth-inhibitory activity at 80 μg/ml. Hyphal growth is partially inhibited at the concentration of 40 μg/ml and below. Larger hyphal diameter is observed in oomycetes grown under exposure with LS with higher number of branches compared to the control.

### Example 10—In Vivo Testing of the Lipopeptide Surfactant on Fish Subjects Infected with Parasites

Three experimental groups are established having 2 tanks per group each containing 5 fish subjects, divided as follows:
Group A) Control fish (no parasite, no LS)
Group B) Fish infected with parasite (only)
Group C) Fish infected with the parasite and treated with LS

Prior to testing the treatment of fish with the LS, parasites are introduced into the tank water of group B and C. The time from introduction of the parasite to testing treatment effect depends on the parasite species and its life cycle and varies from hours to days. When the infection of the fish by the parasite is established, group C is treated with LS from example 1 and 2.

For infections with ciliates, flagellated protists/dinoflagellates, flatworms, and amoeba, the fish receive a bath exposure of LS at the effective concentration (based on the results from MIC tests) for 6 h.

Afterwards, fish are transferred to a tank with plain water. After the treatment, the fish subjects are euthanized and checked under a stereomicroscope for the infection status in the gills, fins, and skin.

### Results

After the course of treatment, fish subjects in group C has no detectable parasite infection, while fish in group B had parasite infection.

### Example 11—In Vivo Testing of the Lipopeptide Surfactant on Fish Subjects Infected with Bacteria

Three experimental groups are established having 2 tanks per group each containing 5 fish subjects, divided as follows:
Group A) Control fish (no bacterium, no LS)
Group B) Fish infected with bacterium (only)
Group C) Fish infected with the bacterium and treated with LS

Prior to testing the treatment of bacterium-infected fish with the LS, the fish in group B and C are challenged by bath exposure to the bacteria in 5 L water volume for 6 h, and then, the tank water level is raised up to 20 L diluting the bacterial concentration. The fish swim in the bacterial solution for 18 h after which water is totally replaced with bacterium free water. Subsequently, fish from group C are subjected to daily baths of 2-3 hours with LS from example 1 and 2 at the effective concentration for 4 days, consecutively.

Upon termination of the experiment, headkidney swabs from fish in group B and C are collected and cultured on blood agar to re-isolate the bacteria from group B to confirm that the bacterium causing disease was identical to the challenge strain, and also to confirm the absence of bacterial infection in treated fish with LS.

### Results

After the course of treatment, no bacterial infection is detected in the fish treated with LS.

### Example 12—In Vivo Testing of the Lipopeptide Surfactant on Subject Infected with Oomycetes

For infection model with oomycete (Saprolegnia/Achlya) salmon eggs are used. Live eggs are placed in 97-98° C. distilled water and incubated for 80-150 s. The dead eggs are drained and placed on potato dextrose agar plates previously grown with oomycete isolates and incubated overnight at 25° C. Afterwards, the eggs are transferred into a 24-well cell culture plate and incubated for 1 day at 15-25° C. until colonization of oomycete hyphae is visible.

Five experimental groups are divided as follows:
Group A) Control salmon eggs (no oomycete, no LS).
Group B) salmon eggs infected with oomycete only (negative control).
Group C) salmon eggs infected with oomycete and treated with LS from example 1 and 2 (concentration 40 μg/ml)
Group D) salmon eggs infected with oomycete and treated with LS from example 1 and 2 (concentration 80 μg/ml)
Group E) salmon eggs infected with oomycete and treated with malachite green (positive control)

Each experimental group is divided in two incubation units each containing three perforated cups with ≈50 live salmon eggs per cup. In the infection groups, two of the dead infected eggs are added to each cup. The infected eggs receive treatment with LS and malachite green every 2-3 days for 90-120 min with aeration. Then, the treated water is removed, eggs are rinsed, and fresh water is replaced. Hyphal expansion on eggs is measured every 48 h and hyphal attachment is evaluated by lifting infection inocula and counting the number of eggs attached to the hyphal patch at 18 days post infection (Liu et al. 2014, Liu et al. 2015).

### Results

Exposure to LS showed partial growth-inhibitory activity at 40 μg/ml and almost complete inhibitory effect at 80 μg/ml.

### Example 13—In Vivo Testing of Lipopeptide Surfactant on Harmful Marine Algal Species

Lipopeptide biosurfactant of DSMZ 34058 (hereinafter referred to as LP34058) obtained in example 3 was tested for effectiveness in inhibiting the growth rate and cell yield of marine harmful microalgae species. The tests were conducted on the microalgae strains following the basic outline of OECD Method No. 201, incorporated herein by reference. Briefly, a single parent culture for each strain, except for Gambierdiscus toxicus (Strain no.: CCMP3466), was grown to mid/late exponential phase in a 500 mL glass flask, this single culture was divided equally across 12 sterile glass test tubes; triplicate test tubes with maintenance media (as a control) and triplicate test tubes with LP34058 in maintenance media at each of ˜16.7, ˜66.7 and ˜333.3 mg/L (active Lipopeptide biosurfactant concentrations of ˜5, ˜20 and ˜100 mg/L concentrations) with a final volume of 18 mL for each replicate. CCMP3466 does not grow well in glass test tubes, and thus grown in a 500 mL glass flask and transferred to sterile 50 mL slant neck tissue culture flasks laying on their side for the test experiment. Culture test tubes/t-flasks were then incubated at the standard growth temperature and irradiance (50-100 μmol photons m-2 s-1) for each strain. Cell growth was assessed by daily readings of in vivo chlorophyll fluorescence (except CCMP3466) and direct counts of cell abundance for 96 h. Cell abundance samples were counted using an Improved Neubauer Hemocytometer, with ˜200 cells counted for each time point.

Growth rates from the in vivo chlorophyll fluorescence and daily cell count estimates were calculated over the entire experimental period using a standard equation (eq. 1) for each replicate in the control and treatments.

$$mu \frac{LN ( \frac{N 3}{N 1} )}{( T 2 - T 1 )} \qquad ( Eq . 1 )$$

where N2 and N1 are fluorescence readings/cell counts at times T2 (96 h) and T1 (0 h). Time course plots of fluorescence and cell counts were plotted and examined to confirm that the control treatment for all phytoplankton strains were growing exponentially throughout the entire test period.

Cell yield, final minus initial cell abundance, was calculated for each strain, as the other evaluation metric noted in OECD Method No. 201. For each strain, differences between the cell count based growth rates and cell yield in each treatment versus the respective control were statistically assessed using a 1-way ANOVA followed by Tukey's test if the normality assumption was met, or the Holm-Sidak Test if it was not. All pairwise comparisons were evaluated against a probability value of 0.05.

### Treatments:

LP34058 was fully dissolved in Milli-Q water to make a 10.04 mg/ml stock solution. The LP34058 solution was sterile filtered (0.2 μm syringe filter) and added to the test tube containing each strain as outlined in Table 13-1.

TABLE 13-1
Calculated concentration of LP34058 in each
experimental treatment.

| [Stock] | Vol. | Vol. | |
|---|---|---|---|
| mg/ml | Stock | culture | [LP34058-trtmt] |
| Q-water | (ml) | (ml) | mg/L |
| 10.04 | 0.6 | 18 | 334.52 |
| 10.04 | 0.12 | 18 | 66.90 |
| 10.04 | 0.03 | 18 | 16.73 |

### Strains Used:

The following marine phytoplankton strains from the NCMA Culture Collection were used in these growth assays (Table 13-2).

TABLE 13-2
List of test strains, growth temperatures and media formulation
to be used in this test experiment.

| | | Growth | | |
|---|---|---|---|---|
| | Taxonomic | Temp. | | NCMA |
| Genus species | affiliation | (° C.) | Medium | strain ID |
| Alexandrium | Dinophyceae | 14 | L1-Si | CCMP1771 |
| tamarense | | | | |
| Aureococcus | Pelagophyceae | 14 | L1-Si | CCMP1984 |
| anophagefferens | | | | |
| Gambierdiscus | Dinophyceae | 24 | Modified | CCMP3466 |
| toxicus | | | K | |
| Heterosigma | Raphidophyceae | 14 | L1-Si | CCMP3149 |
| akashiwo | | | | |
| Karenia brevis | Dinophyceae | 24 | L1-Si | CCMP2281 |
| Prymnesium | Prymnesiophyceae | 14 | L1-Si | CCMP3037 |
| parvum | | | | |

### Results:

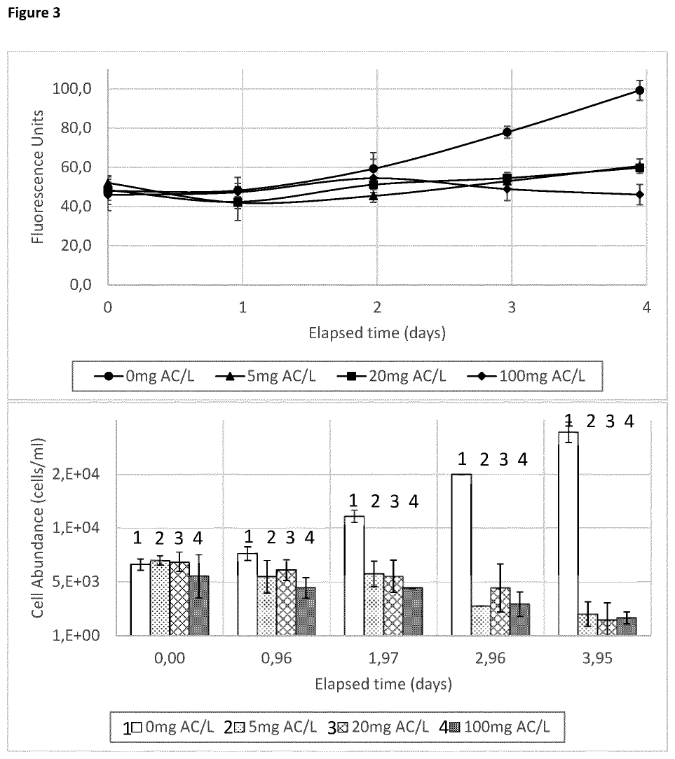

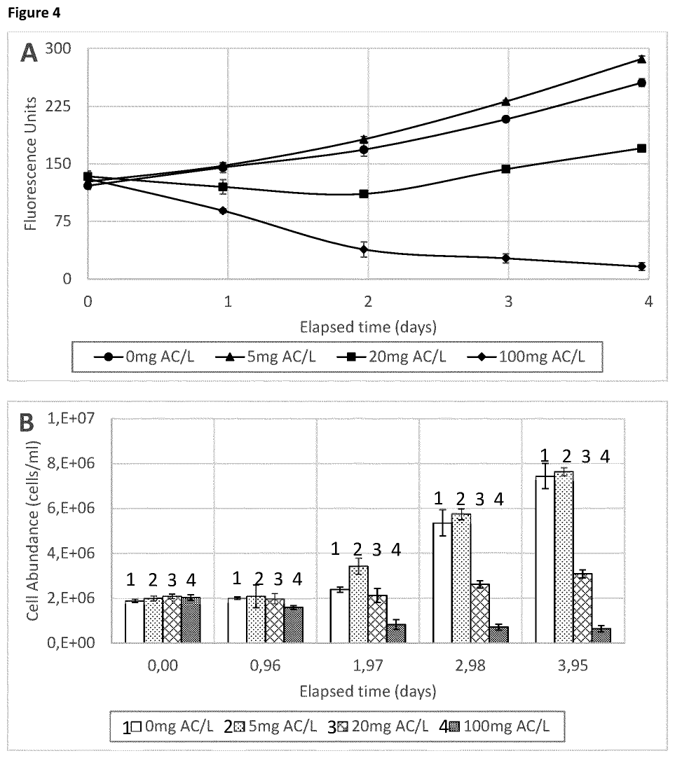

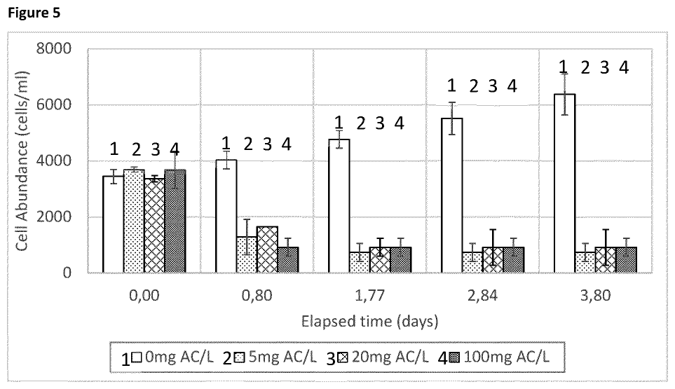

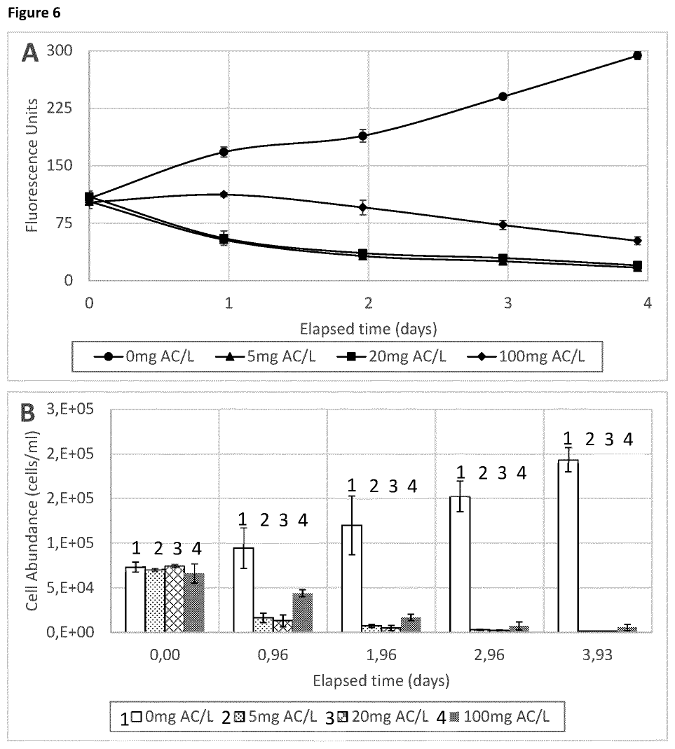

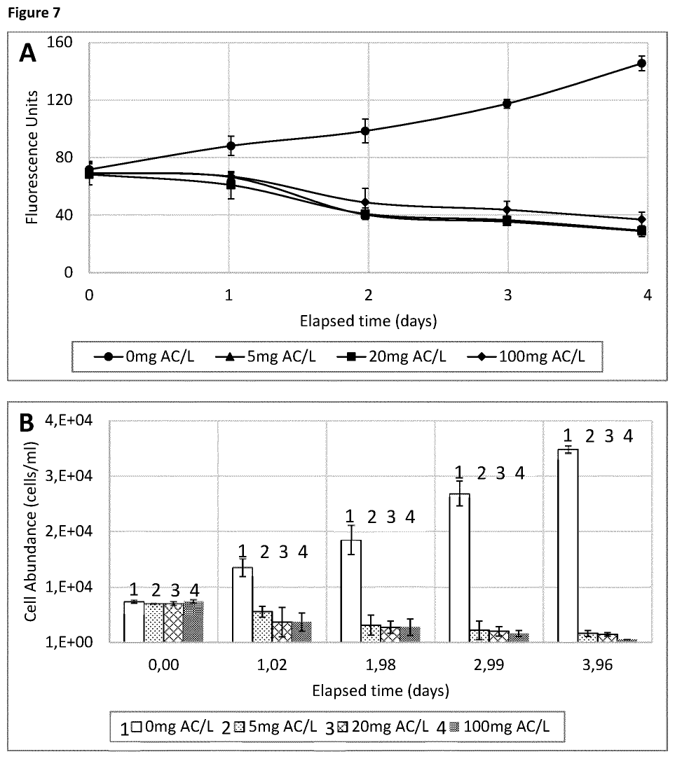

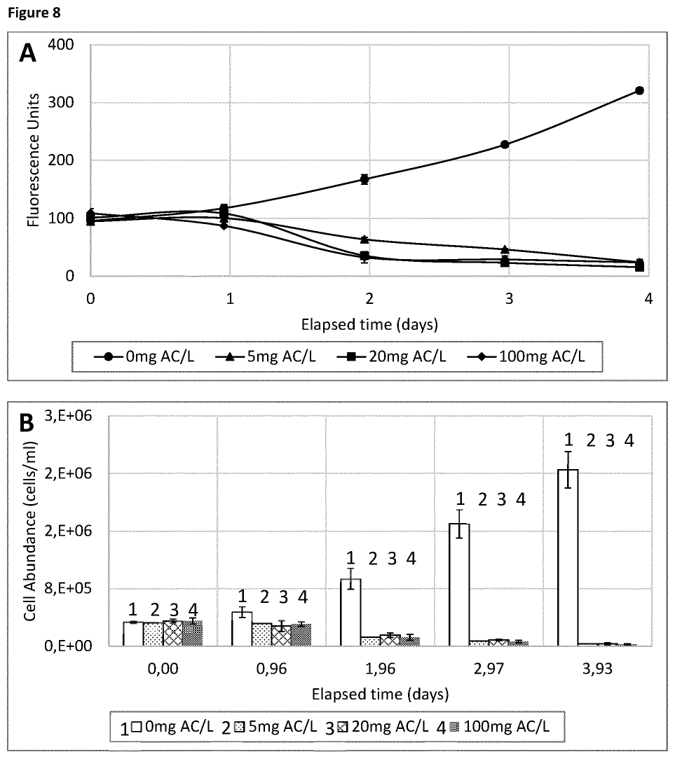

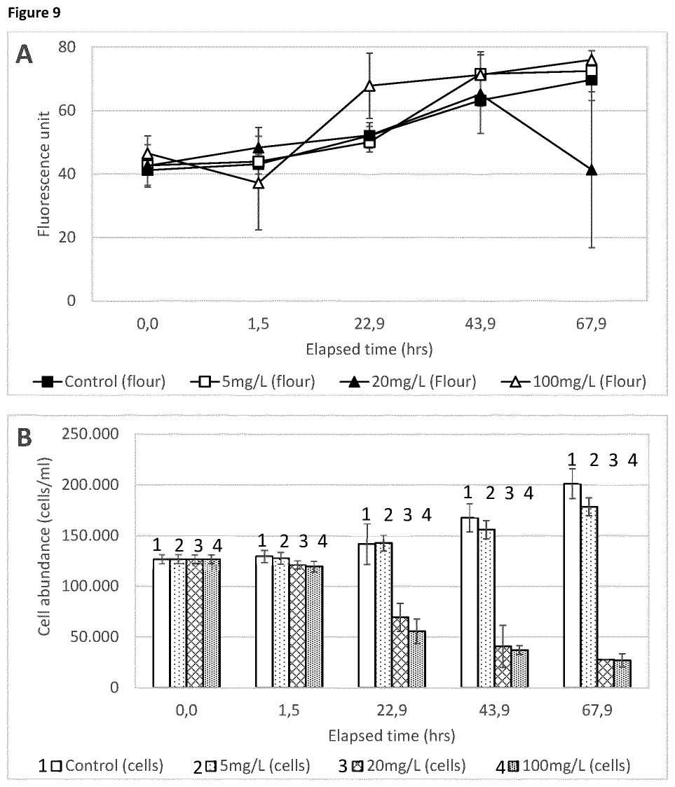

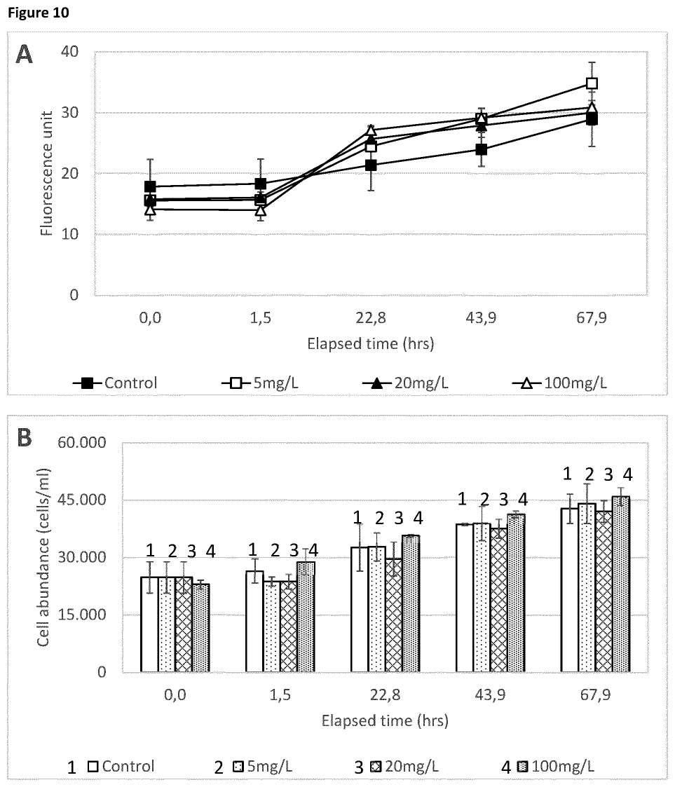

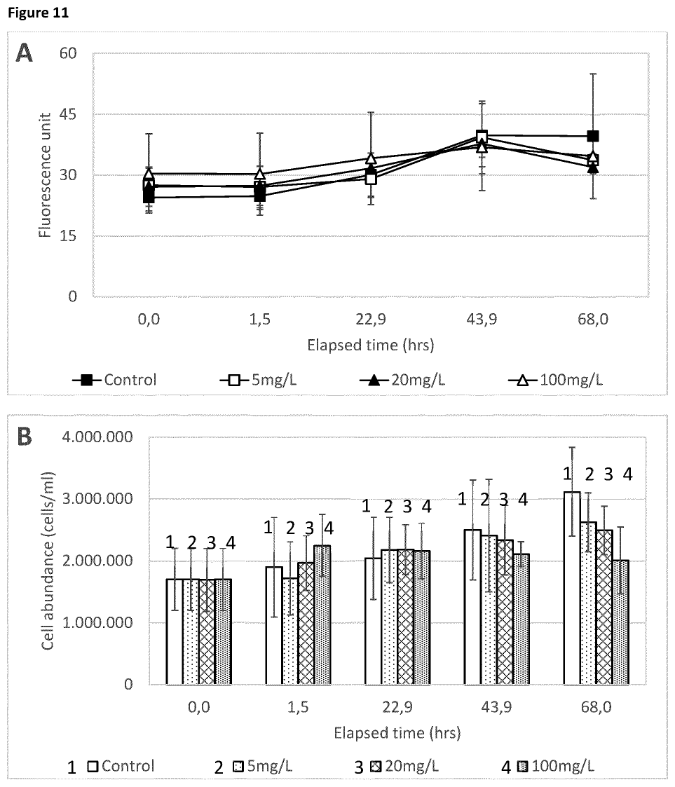

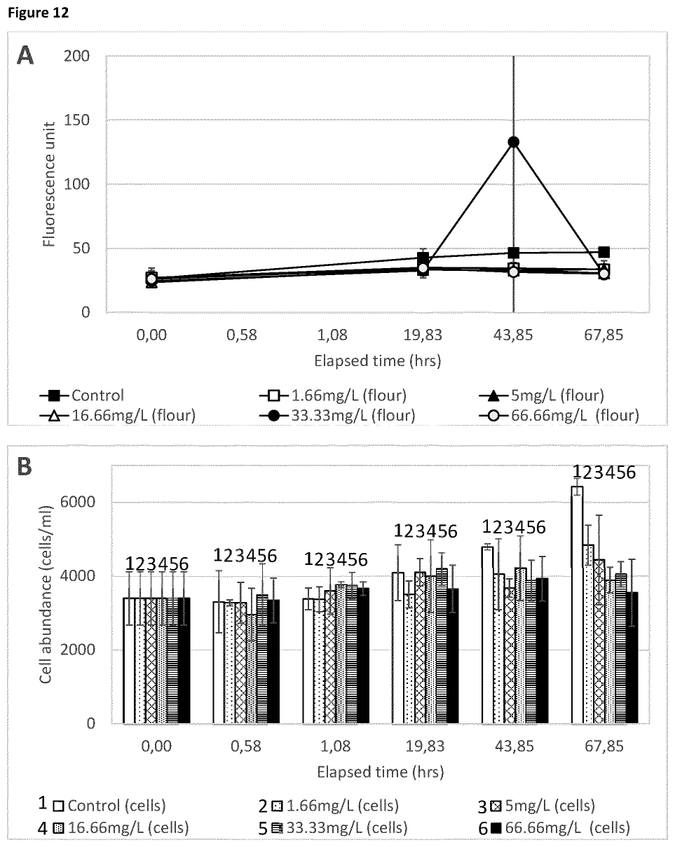

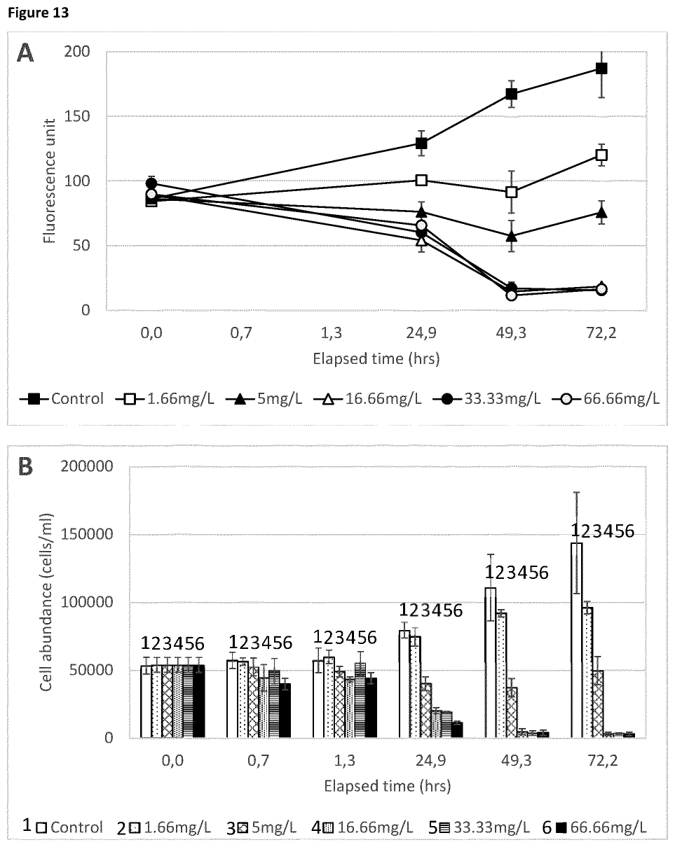

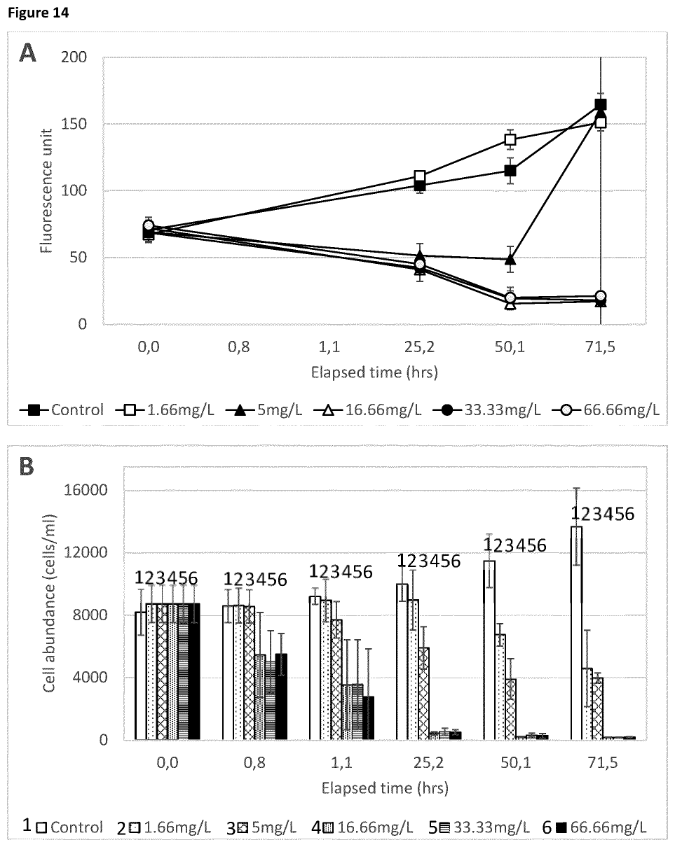

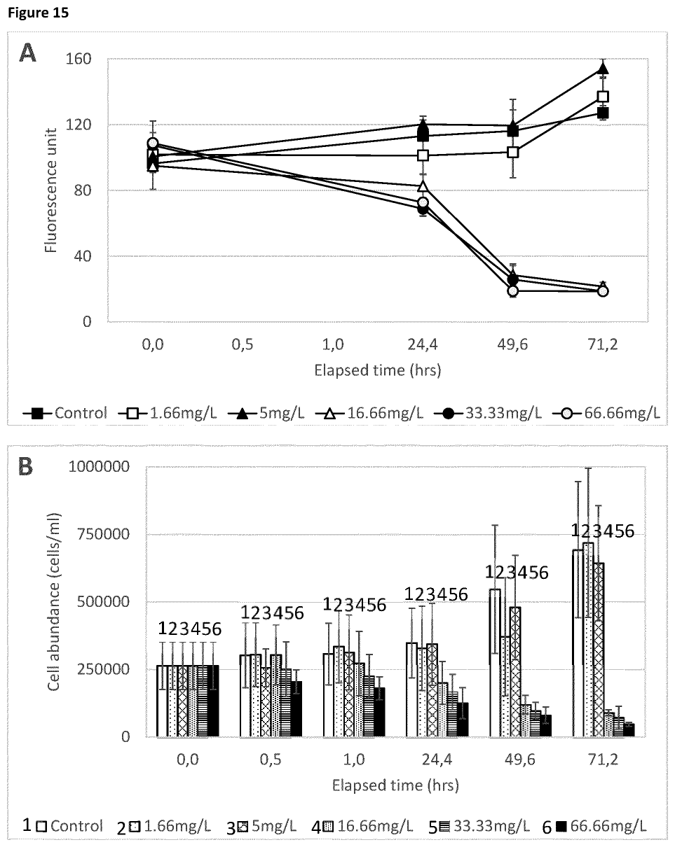

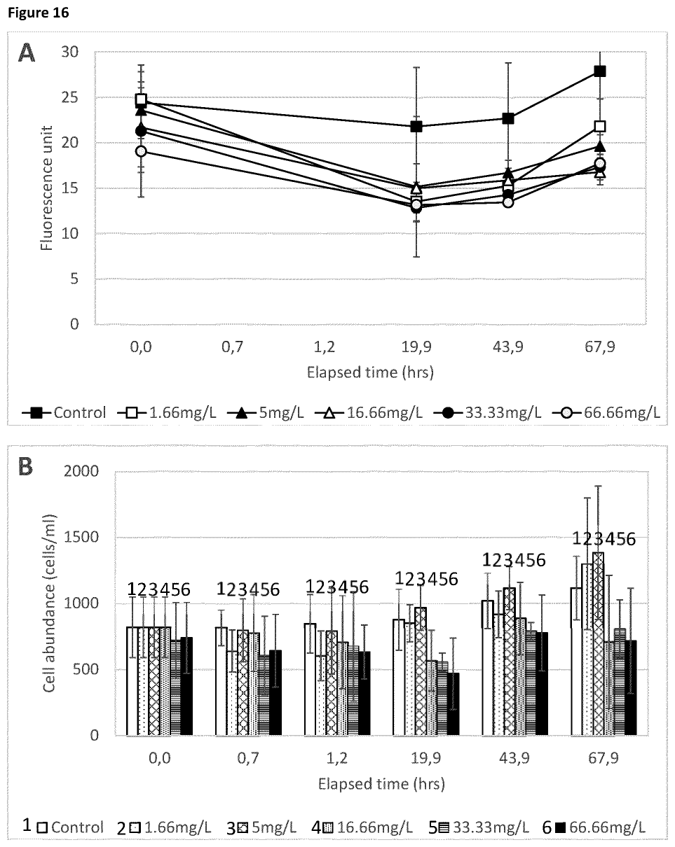

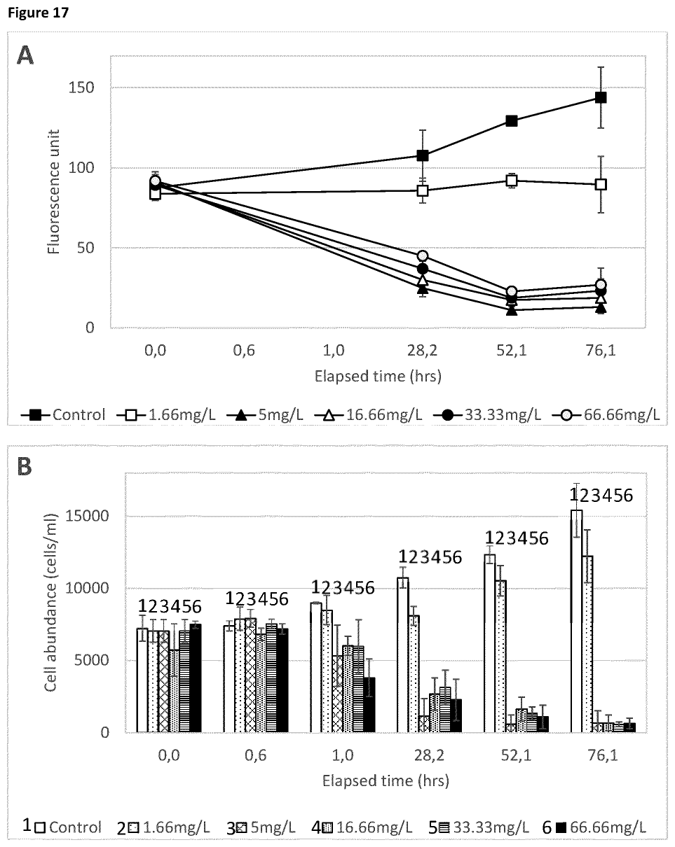

All strains grew in the control treatments. Based upon the examination of the time course plots, all strains were in exponential growth throughout the experiment (ie., linear increase in fluorescence/cell count with time when plotted as the natural log). Growth rates were all significantly greater than zero (Table 13-2). As predicted, growth rates were much lower than those of the ‘green weeds’ recommended in the OECD 201 method, and thus fold-increases in biomass ranged from 1.8-7.4, averaging 3.9. Gambierdiscus toxicus (CCMP3466) grew the slowest and showed the least fold-increase in biomass (1.84), regardless, it was very clear that the LP34058 treatments had the desired effect on this strain. Responses to the LP34058 treatment varied between strains, but all strains were susceptible to LP34058 with at least one treatment concentration, showing a lethal effect and loss of cells. FIG. 3-8 shows in vivo chlorophyll fluorescence (Graph A) and cell abundance (Graph B) of the six marine phytoplankton strains exposed to the three different LP34058 concentration over the test period of 4 days.

### Growth Response:

For all strains, a consistent growth pattern was observed between in vivo chlorophyll fluorescence and cell abundance time courses in the controls and treatments (FIG. 3-8). The only minor exception was for CCMP1771 (Alexandrium tamarense) which showed a reduced, relative to the control, but stable chlorophyll time course in the LP34058 treatments. This suggests that the chlorophyll per cell was increasing over time while the cell number was slowly declining, however the effect of the LP34058 treatment was still quite evident as the cultures did not grow and cell yield ultimately declined (FIG. 3 A, B).

For most strains, even the lowest LP34058 treatment concentration, 16.67 mg LP34058/L & 5 mg active component/L, resulted in a significant reduction in growth rate and cell yield (Table 13-3), the one exception being Aerococcus anophagefferens (CCMP1984). A. anophagefferens did not show any negative impact at the lowest LP34058 concentration, while the mid-level concentration appeared to be sublethal as the culture recovered in the middle of the test and continued to grow but at a slower rate. The highest LP34058 concentration was clearly toxic resulting in significant cell loss. It is worth noting that the majority of the treatments were found to have large negative growth rates, meaning cells were not only not growing, but their cell membrane integrity was being compromised leading to the lysis of cells and thus the dramatic loss of cells; near quantitative loss in many cases. This was very evident for G. toxicus and A. tamarense as few live cells were observed in the LP34058 treatments, but empty thecae were very abundant confirming that there had been live cells in the culture at the beginning of the experiment. For H. akashiwo, K. brevis, and P. parvum the cells become increasingly ‘ratty’ looking, with cell membranes clearly being compromised and the substantial increase in detritus associated with the lysis of cells. Furthermore, for most strains, the LP34058 treatments led to increased settling of cells in the test tube compared to the respective controls, and in some cases, it was very difficult to get the cells resuspended. Overall, it appears that the LP34058 compound showed the desired response—significant reduction in harmful algae growth and cell abundance. The reduced data are shown in Table 13-3, and includes calculated growth rates for controls and treatments, percent inhibition calculations, and statistical analyses.

TABLE 13-3
Summary statistics of fluorescence-based growth rate (FL mu), cell count-
based growth rate (CC mu), and cell yield at 96h. CC mu inhibition and
yield inhibition were assessed relative to the respective control.

| | | | | | CC mu | | Yield | |
|---|---|---|---|---|---|---|---|---|
| | Treatment | FL mu | CC mu | yield | inhibition | P- | Inhibition | P- |
| Strain | (mg/L) | (/d) | (/d) | (c/ml) | (%) | value | (%) | value |
| CCMP | Control | 0.18 | 0.26 | 18900 | n/a | n/a | n/a | n/a |
| 1771 | 16.67 | 0.04 | −0.34 | 2017 | −228.1 | 0.002 | −89.5 | 0.002 |
| | 66.67 | 0.07 | −0.48 | 1467 | −282.9 | <0.001 | −92.4 | <0.001 |
| | 333.33 | 0.00 | −0.30 | 1650 | −214.0 | 0.003 | −91.3 | 0.003 |
| CCMP | Control | 0.19 | 0.34 | 7625000 | n/a | n/a | n/a | n/a |
| 1984 | 16.67 | 0.20 | 0.33 | 7500000 | −1.6 | NS | 2.9 | NS |
| | 66.67 | 0.06 | 0.10 | 3100000 | −71.2 | <0.05 | −58.3 | <0.05 |
| | 333.33 | −0.52 | −0.29 | 602500 | −185.8 | <0.05 | −91.3 | <0.05 |
| CCMP | Control | n/d | 0.15 | 6375 | n/a | n/a | n/a | n/a |
| 3466 | 16.67 | n/d | −0.42 | 733 | −383.1 | <0.001 | −88.2 | <0.001 |
| | 66.67 | n/d | −0.36 | 917 | −360.0 | <0.001 | −86.2 | <0.001 |
| | 333.33 | n/d | −0.36 | 917 | −333.8 | <0.001 | −85.1 | <0.001 |
| CCMP | Control | 0.25 | 0.24 | 193483 | n/a | n/a | n/a | n/a |
| 3149 | 16.67 | −0.46 | −0.94 | 1650 | −488.8 | <0.001 | −99.1 | <0.001 |
| | 66.67 | −0.44 | −0.99 | 1467 | −508.8 | <0.001 | −99.2 | <0.001 |
| | 333.33 | −0.17 | −0.65 | 5533 | −366.5 | <0.001 | −97.2 | <0.001 |
| CCMP | Control | 0.18 | 0.39 | 34817 | n/a | n/a | n/a | n/a |
| 2281 | 16.67 | −0.22 | −0.37 | 1650 | −195.7 | <0.05 | −95.3 | <0.05 |
| | 66.67 | −0.21 | −0.40 | 1467 | −202.2 | <0.05 | −95.8 | <0.05 |
| | 333.33 | −0.16 | −0.65 | 550 | −267.7 | <0.05 | −98.4 | <0.05 |
| CCMP | Control | 0.30 | 0.50 | 2300000 | n/a | n/a | n/a | n/a |
| | 16.67 | −0.34 | −0.60 | 29550 | −220.4 | <0.001 | −98.7 | <0.001 |
| | 66.67 | −0.47 | −0.61 | 29917 | −223.2 | <0.001 | −98.7 | <0.001 |
| | 333.33 | −0.39 | −0.66 | 23783 | −232.1 | <0.001 | −99.0 | <0.001 |

n/a = not applicable, controls were not compared to themselves.
NS = not significant.

### Lethal Doses 50% (LD50) Estimation:

LD50 values (i.e. the concentration at which 50% of algal cells will be killed after 96 hrs) were estimated using the AAT Bioquest online calculator2 (incorporated herein by reference), with the results given in Table 13-4. Given that for most strains, all LP34058 treatments results in near total cell loss even at the lowest treatment concentration estimated LD50's should be considered with a great degree of caution.

However, for A. tamarense (CCMP1771) and A. anophagefferens (CCMP1984), these estimates appear reasonably robust.

TABLE 13-4
Summary of calculated LD50 values, based upon cell
number in treatments relative to respective controls
(i.e., a calculated percent survivability value).

| | Calculated |
|---|---|
| | LD50 |
| Strain | (mg/L) |
| A. tamarense (CCMP1771) | 9.8 |
| A. anophagefferens (CCMP1984) | 62.2 |
| G. toxicus (CCMP3466) | 0.3 |
| H. akashiwo (CCMP3149) | 0.4 |
| K. brevis (CCMP2281) | 0.3 |
| P. parvum (CCMP3037) | Calculation failed |

### Example 14 In Vivo Testing of Lipopeptide Surfactant on Harmful Marine Algal and Cyanobacterial Species

Lipopeptide biosurfactant of DSMZ 34058 (hereinafter referred to as LP34058) obtained in example 3 was tested for effectiveness in inhibiting the growth rate and cell yield of marine harmful microalgae species and freshwater/brackish water cyanobacteria species by the National Center for Marine Algae and Microbiota (NCMA) at Bigelow Laboratory. The tests were conducted on the strains following the basic outline of OECD Method No. 201, incorporated herein by reference. Briefly, a single parent culture for each strain was grown to mid/late exponential phase in a 500 mL glass flask, this single culture was divided equally across sterile glass test tubes; triplicate test tubes with maintenance media (as a control) and triplicate test tubes with LP34058 in maintenance media at each of concentrations listed in Table 14-1. Culture test tubes/t-flasks were then incubated at the standard growth temperature and irradiance (50-100 μmol photons m-2 s-1) for each strain. Cell growth was assessed by readings of in vivo chlorophyll fluorescence and direct counts of cell abundance for 72 h.

Cell abundance samples were counted using an Improved Neubauer Hemocytometer or a Palmer Maloney counting chamber.

Growth rates from the in vivo chlorophyll fluorescence and cell count estimates were calculated over the entire experimental period using a standard equation (eq. 1) for each replicate in the control and treatments.

$$mu \frac{LN ( \frac{N 3}{N 1} )}{( T 2 - T 1 )} \qquad ( Eq . 1 )$$

Where N2 and N1 are fluorescence readings/cell counts at times T2 (72 h) and T1 (0 h). Time course plots of fluorescence and cell counts were plotted and examined to confirm that the control treatment for all phytoplankton strains were growing exponentially throughout the entire test period. Cell yield, final minus initial cell abundance, was calculated for each strain, as the other evaluation metric noted in OECD Method No. 201. For each strain, differences between the cell count based growth rates and cell yield in each treatment versus the respective control were statistically assessed using a 1-way ANOVA followed by Tukey's test if the normality assumption was not met, or the Holm-Sidak Test if it was. All pairwise comparisons were evaluated against a probability value of 0.05.

### Treatments:

LP34058 was made up at 10.04 mg/ml in Milli-Q water. The compound fully dissolved and no ‘pellet’ was observed at the bottom and thus no sodium hydroxide was added (per dilution protocol provided by Sundew). The LP34058 solution was sterile filtered (0.2 μm syringe filter) and added to the test tube containing each strain as outlined in Table 14-1.

TABLE 14-1
Calculated concentration of LP34058 in each experimental treatment.
Cyanobacteria testing concentrations are highlighted in bold.

| [Stock] mg/ml | Vol. Stock | Vol. culture | [LP34058-trtmt] |
|---|---|---|---|
| Q-water | (ml) | (ml) | mg/L |
| 10.04 | 0.6 | 18 | 334.52 |
| 10.04 | 0.12 | 18 | 66.90 |
| 10.04 | 0.03 | 18 | 16.73 |
| 10.04 | 0.12 | 18 | 66.90 |
| 10.04 | 0.06 | 18 | 33.45 |
| 10.04 | 0.03 | 18 | 16.73 |
| 10.04 | 0.01 | 18 | 5.58 |
| 10.04 | 0.003 | 18 | 1.67 |

### Strains Used:

The following marine phytoplankton strains from the NCMA Culture Collection were used in these growth assays (Table 14-2).

TABLE 14-2
List of test strains, growth temperatures and media
formulation to be used in this test experiment.

| | | | | |
|---|---|---|---|---|
| | Taxonomic | Growth Temp. | | |
| Genus species | affiliation | (° C.) | Medium | NCMA strain ID |

LD50 resolution: Marine Strains to be tested at 1.66, 5, 16.66, 33.33 and 66.66 mg LP34058/L
(active compound concentration of 0.5, 1.5, 5, 10 and 20 mg/L, respectively),
with data recorded at 0, 0.5, 1, 24, 48, 72 hrs after the addition of LP34058.

| | | | | |
|---|---|---|---|---|
| Alexandrium | Dinophyceae | 14 | L1-Si | CCMP1771 |
| tamarense | | | | |
| Heterosigma | Raphidophyceae | 14 | L1-Si | CCMP3149 |
| akashiwo | | | | |
| Karenia brevis | Dinophyceae | 24 | L1-Si | CCMP2281 |
| Prymnesium parvum | Prymnesiophyceae | 14 | L1-Si | CCMP3037 |
| Prorocentrum lima | Dinophyceae | 24 | L1-Si | CCMP684 |
| Chattonella marina | Raphidophyceae | 24 | L1-Si | CCMP2962 |

New Test Spp.: Freshwater cyanobacteria strains to be tested at 16.66, 66.66 and 333.3 mg
LP34058/L (active compound concentrations of 5, 20 and 100 mg/L, respectively),
with data recorded at 0, 1, 24, 48, 72 hrs after the addition of LP34058.

| | | | | |
|---|---|---|---|---|
| Nostoc sp. | Cyanobacteria | 20 | Black Sea | CCMP3413 |
| Aphanizomenon sp. | Cyanobacteria | 14 | DYV-m | CCMP2764 |
| Microcystis cf. | Cyanobacteria | 20 | AF6 | CCMP3462 |
| aeruginosa | | | | |

### Results:

All strains grew in the control treatments, although growth rates were low to moderate. Based upon the examination of the time course plots, all strains were in exponential growth throughout the experiment (ie., linear increase in fluorescence/cell count with time when plotted as the natural log). As predicted, growth rates were much lower than those of the ‘green weeds’ recommended in the OECD 201 method, and thus fold-increases in biomass in the control treatments was <3-fold. Responses to the LP34058 treatment varied between strains, but all strains were susceptible to LP34058 with at least one treatment concentration, showing a lethal effect and loss of cells (Tables 14-3 & 14-4).

### General Growth Response:

In this experiment, with the finer temporal resolution of 0.5-1 h measurements highlighted that there appeared to be an autofluorescence in the LP34058 compound. Without further study we cannot determine which compound may be autofluorescing, understand its decay characteristics, and ultimately its impact on fluorescence in the Chlorophyll emission wavelength range. Thus, for this experiment, while we include the chlorophyll a fluorescence data, it should be interpreted with care.

For most strains in the ‘low test concentration’ range, even the lowest LP34058 treatment concentration resulted in a substantial reduction in growth rate and cell yield (Table 14-4). The same was not the case with the cyanobacteria (Table 14-3), where two of the three strains tested did not show a significant impact of the LP34058 treatment. Why these two strains were not sensitive to the LP34058 compound is unknown. For CCMP2764 (Nostoc sp.) it appears that the LP34058 compound had the effect of disrupting the compounds holding filaments together, but not the cell walls themselves, thus there was an impact on growth morphology, see next subsection, but not on growth rate.

Higher LP34058 concentrations were clearly toxic resulting in significant cell loss for many non-cyanobacteria strains. It is worth noting that the majority of the treatments were found to have large negative growth rates, meaning cells were not only not growing, but their cell membrane integrity was being compromised leading to the lysis of cells and thus the dramatic loss of cells; near quantitative loss in many cases. This was very evident for A. tamarense as few live cells were observed, and counted, in the LP34058 treatments but empty thecae were very abundant confirming that there had been live cells in the culture at the beginning of the experiment. For H. akashiwo, K. brevis, and P. parvum the cells become increasingly ‘ratty’ looking, with cell membranes clearly being compromised and the substantial increase in detritus associated with the lysis of cells. Furthermore, for most non-cyanobacteria strains, the LP34058 treatments led to increased settling of cells in the test tube compared to the respective controls, and in some cases, it was very difficult to get the cells resuspended. Overall, it appears that the LP34058 compound showed the desired response—significant reduction in harmful algae growth and cell abundance.

The reduced data are shown in Table 14-3 and 14-4, and includes calculated growth rates for controls and treatments, percent inhibition calculations, and statistical analyses.

TABLE 14-3
Summary statistics of fluorescence-based growth rate (FL mu), cell count-based
growth rate (CC mu), and cell yield at 72 h for cyanobacteria strains. CC mu inhibition
and yield inhibition were assessed relative to the respective control.

| | Treatment | FL mu | CC mu | yield | CC mu | | Yield Inhibition | |
|---|---|---|---|---|---|---|---|---|
| Strain | (mg/L) | (/d) | (/d) | (c/ml) | inhibition (%) | P-value | (%) | P-value |
| CCMP3413 | Control | 0.22 | 0.14 | 167593 | n/a | n/a | n/a | n/a |
| | 16.67 | 0.26 | 0.10 | 155926 | −24.7 | NS | −6.8 | NS |
| | 66.67 | 0.20 | −0.62 | 40741 | −531.1 | <0.05 | −74.9 | <0.05 |
| | 333.33 | 0.21 | −0.62 | 37037 | −557.7 | <0.05 | −77.8 | <0.05 |
| CCMP2764 | Control | 0.15 | 0.23 | 38704 | n/a | n/a | n/a | n/a |
| | 16.67 | 0.31 | 0.23 | 38889 | 0.8 | NS | 0.4 | NS |
| | 66.67 | 0.28 | 0.21 | 37593 | −6.9 | NS | −2.9 | NS |
| | 333.33 | 0.37 | 0.29 | 41296 | 39.5 | NS | 6.7 | NS |
| CCMP3462 | Control | 0.24 | 0.19 | 2500000 | n/a | n/a | n/a | n/a |
| | 16.67 | 0.17 | 0.16 | 2408333 | −34.2 | NS | −4.7 | NS |
| | 66.67 | 0.17 | 0.17 | 2333333 | −1.9 | NS | −4.6 | NS |
| | 333.33 | 0.10 | 0.12 | 2108333 | −15.4 | NS | −9.4 | NS |

n/a = not applicable, controls were not compared to themselves.
NS = not significant.

TABLE 14-4
Summary statistics of fluorescence-based growth rate (FL mu), cell count-based growth
rate (CC mu), and cell yield at 72 h for non-cyanobacteria strains. CC mu inhibition
and yield inhibition were assessed relative to the respective control.

| | Treatment | FL mu | CC mu | yield | CC mu | | Yield Inhibition | |
|---|---|---|---|---|---|---|---|---|
| Strain | (mg/L) | (/d) | (/d) | (c/ml) | inhibition (%) | P-value | (%) | P-value |
| CCMP | Control | 0.20 | 0.22 | 6427 | n/a | n/a | n/a | n/a |
| 1771 | 1.66 | 0.07 | 0.12 | 4840 | −48.4 | NS | −24.7 | NS |
| | 5 | 0.08 | 0.09 | 4440 | −64.2 | NS | −30.5 | NS |
| | 16.66 | 0.09 | 0.05 | 3893 | −81.3 | NS | −39.5 | NS |
| | 33.33 | 0.06 | 0.06 | 4053 | −79.0 | NS | −36.8 | NS |
| | 66.66 | 0.05 | 0.01 | 3553 | −106.3 | NS | −44.7 | NS |
| CCMP | Control | 0.26 | 0.32 | 143704 | n/a | n/a | n/a | n/a |
| 3149 | 1.66 | 0.12 | 0.19 | 95926 | −39.3 | <0.05 | −30.6 | <0.05 |
| | 5 | −0.04 | 0.27 | 49630 | −18.7 | <0.05 | −64.2 | <0.05 |
| | 16.66 | −0.53 | −0.98 | 2963 | −429.9 | <0.05 | −98.0 | <0.05 |
| | 33.33 | −0.62 | −0.93 | 3333 | −406.3 | <0.05 | −97.6 | <0.05 |
| | 66.66 | −0.57 | −0.98 | 2963 | −429.9 | <0.05 | −98.0 | <0.05 |
| CCMP | Control | 0.28 | 0.17 | 13667 | n/a | n/a | n/a | n/a |
| 2281 | 1.66 | 0.27 | −0.24 | 4587 | −242.5 | <0.05 | −64.7 | <0.05 |
| | 5 | 0.13 | −0.26 | 3973 | −317.9 | <0.05 | −70.3 | <0.05 |
| | 16.66 | −0.48 | −1.31 | 173 | −1115.5 | <0.05 | −98.7 | <0.05 |
| | 33.33 | −0.45 | −1.31 | 173 | −1092.1 | <0.05 | −98.7 | <0.05 |
| | 66.66 | −0.42 | −1.28 | 187 | −1077.7 | <0.05 | −98.6 | <0.05 |
| CCMP | Control | 0.09 | 0.32 | 693333 | n/a | n/a | n/a | n/a |
| 3037 | 1.66 | 0.10 | 0.33 | 719167 | 3.2 | NS | 3.4 | NS |
| | 5 | 0.14 | 0.30 | 643333 | −6.6 | NS | −6.1 | NS |
| | 16.66 | −0.49 | −0.35 | 90000 | −207.5 | <0.05 | −85.9 | <0.05 |
| | 33.33 | −0.58 | −0.45 | 72083 | −242.8 | <0.05 | −89.5 | <0.05 |
| | 66.66 | −0.59 | −0.56 | 47917 | −274.1 | <0.05 | −92.5 | <0.05 |
| CCMP | Control | 0.03 | 0.11 | 1117 | n/a | n/a | n/a | n/a |
| 684 | 1.66 | −0.04 | 0.15 | 1300 | 69.4 | NS | 15.1 | NS |
| | 5 | −0.06 | 0.17 | 1383 | 93.9 | NS | 23.5 | NS |
| | 16.66 | −0.08 | −0.13 | 710 | −368.5 | <0.05 | −33.2 | <0.05 |
| | 33.33 | −0.06 | 0.04 | 807 | −127.1 | <0.05 | −24.1 | <0.05 |
| | 66.66 | −0.02 | −0.05 | 717 | −272.8 | <0.05 | −30.9 | <0.05 |
| CCMP | Control | 0.16 | 0.25 | 15400 | n/a | n/a | n/a | n/a |
| 2962 | 1.66 | 0.02 | 0.18 | 12213 | −27.9 | NS | −19.1 | NS |
| | 5 | −0.65 | −0.96 | 680 | −479.8 | <0.05 | −95.8 | <0.05 |
| | 16.66 | −0.52 | −0.82 | 680 | −425.9 | <0.05 | −95.8 | <0.05 |
| | 33.33 | −0.49 | −0.84 | 587 | −432.9 | <0.05 | −96.1 | <0.05 |
| | 66.66 | −0.41 | −0.86 | 653 | −443.5 | <0.05 | −95.6 | <0.05 |

n/a = not applicable, controls were not compared to themselves.
NS = not significant.

### Specific Growth Response:

In this section, we briefly summarize strain specific growth observations.

CCMP2962—this strain became very sticky in the LP34058 treatments, perhaps due to release of polysaccharides upon the cell membrane being compromised. As a result, the culture would form very large aggregates, even if the cells were still clearly cells, making it very difficult to count cells accurately. In the higher concentrations, those aggregates became increasingly difficult to resuspend.

CCMP3413 & CCMP2764—these strains are filamentous cyanobacteria making them difficult to count even in the control (filament lengths vary a little bit), but in the LP34058 treatments, the filaments all were disrupted. So, while in the control there were 15-20 cells/filament, in the LP34058 treatments, it was pretty consistent that filaments were disrupted into pairs of cells. Thus, to assess ‘growth’ required a lot more counting of partial filaments and scaling those back to the whole filaments in the control samples. The counts are presented as filaments/mi not cells/mi.

CCMP2281—this strain is of note as the LP34058 treatments led to very rapid cell lysis at >1 mg/L active ingredient concentration.

CCMP1771—this strain settled to the bottom of the culture tube in the LP34058 treatments but not the control, an observation made in the prior analysis, although it appeared more pronounced in this experiment. Perhaps this is why it was less sensitive in this experiment than in the prior experiment, as cells looked generally healthy when resuspended.

### LD50 Estimation:

LD50 values for growth inhibition were estimated using the AAT Bioquest online calculator2, with the results given in Table 14-5. Given that for most strains, all treatments results in significant cell loss estimated LD50's should be considered with caution. LD50s are presented as concentration of the active ingredient in the LP34058. Values for all but one strain tested at the low-test concentration range resulted in LD50 values that could be calculated. The dinoflagellate Prorocentrum lima showed substantial reduction in growth, but the resulting LD50 exceeded the test concentration range and should be considered qualitative. For the cyanobacteria, an LD50 could be calculated for Nostoc sp., but not for Aphanizomenon sp. While an LD50 could be calculated for M. aeruginosa, the value exceeded the test concentration range and thus should be considered qualitative.

For some of the strains, this experiment was a retest at a lower concentration range. For those strains, we combined data from both experiments to estimate an LD50 value. For CCMP1771 & CCMP2281, the calculated LD50s have a significant degree of uncertainty. For CCMP1771, it was less sensitive than in the first experiment, and for CCMP2281 it was more sensitive than in the first experiment. CCMP3149 & CCMP3037 showed very good agreement between the experiments.

TABLE 14-5
Summary of calculated LD50 values,
based upon cell number in treatments
(active ingredient concentration) relative
to respective controls
(i.e., a calculated percent survivability value).
Cyanobacteria are highlighted in bold.

| | Calculated | Calculated |
|---|---|---|
| | LD50 | LD50 |
| Strain | (mg/L)* | (mg/L)** |
| A. tamarense (CCMP1771) | 12.7 | 5.7 |
| H. akashiwo (CCMP3149) | 2.2 | 2.0 |
| K. brevis (CCMP2281) | 1.5 | 0.8 |
| P. parvum (CCMP3037) | 3.1 | 2.3 |
| P. lima (CCMP684) | 712.5 | |
| C. marina (CCMP2962) | 0.6 | |
| Nostoc sp. (CCMP3413) | 7.9 | |
| Aphanizomenon sp. | No reasonable | |
| (CCMP2764) | LD50 | |
| M. aeruginosa(CCMP3462) | 139.6 | |

*this experiment only.
**from both the experiment described in Example 12 and this experiment - only calculated for strains used in both experiments.

### Example 15—In Vitro Testing of the Lipopeptide Surfactant Effect on Ciliates

Lipopeptide biosurfactant of DSMZ 34058 (hereinafter referred to as LP34058) obtained in example 3 was used to test the effects on Chilodonella uncinata reproduction and survival using in vitro bioassays.

Survival and growth of protozoa Chilodonella uncinata (C. uncinata) under assay conditions was observed for, at minimum, 24 hours prior to assay start. Peak growth time points for C. uncinata cultures were validated prior to assay start. Cultures of C. uncinata were incubated at 15° C. and split three days prior to assay start. Cultures were observed microscopically for peak growth phase confirmation immediately prior to sampling. Samples were taken from culture surface and 100 μL was pipetted in quadruplicate into six rows of a transparent 96-well microtitre plate. The number of C. uncinata was observed microscopically and recorded for each well. Individual wells had to contain, at minimum, 15 C. uncinata to be utilized for the assay and wells containing fewer C. uncinata were replaced with an additional replicate.

A stock solution of 10 mg/ml LP34058 was prepared utilizing Sonneborn's Paramecium Media as a diluent, immediately prior to assay start. Stock solution was diluted to twice the effective concentrations listed in Table 15-1. Each LP34058 dilution, and Sonneborn's Paramecium Media alone as a negative control, was pipetted in quadruplicate into a well containing a pre-recorded number of C. uncinata. Number of motile C. uncinata were observed and recorded immediately post-addition of either diluted LP34058 or Sonneborn's Paramecium Media, and at 15, 30, 45, 60, 90, and 120 minutes post-addition. A final observation was performed at 24 hours.

The microtitre plate was incubated at 20° C. between observations. Each count was independently verified between two operators and had to be within ±5 count of one another to be accepted. Only motile C. uncinata were counted.

TABLE 15-1
Nominal concentrations of LP34058.

| | Concen- | Concentration of |
|---|---|---|
| | tration | active compound |
| Dilution | (μg/ml) | (AC) (μg/ml) |
| 1 | 250 | 100 |
| 2 | 125 | 50 |
| 3 | 50 | 20 |
| 4 | 25 | 10 |
| 5 | 12.5 | 5 |

### Results

Concentrations of LP34058 50 μg/ml (20 μg AC/ml), 125 μg/ml (50 μg AC/ml), and 250 μg/ml (100 μg AC/ml) were immediately lethal to C. uncinata and these groups were reduced to zero survivors, which was maintained until assay termination. Exposure to 25 μg/ml (10 μg AC/ml) LP34058 was 79% lethal immediately, and 100% lethal by 15 minutes. Exposure to 12.5 μg/ml (5 μg AC/ml) was 30% lethal immediately, 76% lethal by 15 minutes, and 93% lethal by 30 minutes, after which the remaining survivors ceased to decline and remained stable until 24 hours. Negative control (no LP34058) C. uncinata population increased over the duration of the assay (Average Δ_{count} 68) (Table 15-2).

Survival of C. uncinata was not affected by addition of diluent or by the conditions of the assay, as represented by the increase of the negative control population over 24 hours. Though 7% of C. uncinata exposed to 12.5 μg/ml (5 μg AC/ml) survived for the duration of the assay, efficacy was observed through the 93% decline in viability and the failure of remaining C. uncinata to replicate over a 24-hour period. Therefore, of the tested conditions, the minimum lethal dose of LP34058 against C. uncinata is 25 μg/ml (10 μg AC/ml) and the minimum effective dose is 12.5 μg/ml (5 μg AC/ml).

TABLE 15-2
Observed average count ± standard deviation of motile Chilodonella uncinata
in quadruplicate wells for effective concentrations of LP34058 and
negative control at each observation time point.

| | Average number of Chilodonella uncinata | | | | | |
|---|---|---|---|---|---|---|
| Observation | Negative | | | | | |
| Time (Min) | Control | 250 μg/ml | 125 μg/ml | 50 μg/ml | 25 μg/ml | 12.5 μg/ml |
| Pre-Challenge | 32.0 ± 2.6 | 29.5 ± 3.3 | 25.5 ± 4.7 | 23.5 ± 2.6 | 23.5 ± 4.2 | 31.8± |
| 0 | 32.0 ± 4.1 | 0 | 0 | 0 | 5 ± 3.7 | 22.1± |
| 15 | 40.6 ± 4.1 | 0 | 0 | 0 | 0 | 7.8± |
| 30 | 44.6 ± 5.0 | 0 | 0 | 0 | 0 | 2.3± |
| 45 | 49.6 ± 6.4 | 0 | 0 | 0 | 0 | 1.5± |
| 60 | 54.9 ± 7.7 | 0 | 0 | 0 | 0 | 2.0± |
| 90 | 47.9 ± 2.6 | 0 | 0 | 0 | 0 | 2.0± |
| 120 | 62.8 ± 2.6 | 0 | 0 | 0 | 0 | 1.5± |
| 1440 | >100.0^{a} | 0 | 0 | 0 | 0 | 1.8± |

^{a}Too many to accurately count.

This experiment demonstrated that LP34058, at concentrations between 12.5 (5 μg AC/mi) and 250 μg/ml (100 g AC/mi), was highly efficacious against C. uncinate.

### Example 16—In Vitro Testing of the Lipopeptide Surfactant Effect on Sporozoans

Lipopeptide biosurfactant of DSMZ 34058 (hereinafter referred to as LP34058) obtained in example 3 was tested for biocidal effect on two sporozoans Eimeria and Cryptosporidium species.

### Study Design

The negative control group did not contain LP34058 and enabled observation of the chosen organisms without the test item being present. Two reference items were used which are known to kill the intended species at the manufacturers recommended dosage.

Eimeria

| Treatment | No. | TI Dose | | Reference | | Contact time |
|---|---|---|---|---|---|---|
| group | Replicates | (μg/mL) | | Item Dose | | (hours) |
| Negative Control | 3 | 0 | | Water | | 24 |
| Positive Control | 3 | N/A | | 1:30 | | 24 |
| (Reference Item) | | | | Bi-oo-cyst | | |
| LP34058 (1) | 3 | 17 | (5 AC) | 17 | μg/ml | 24 |
| LP34058 (2) | 3 | 67 | (20 AC) | 67 | μg/ml | 24 |
| LP34058 (3) | 3 | 333 | (100 AC) | 333 | μg/ml | 24 |

Cryptosporidium parvum

| Treatment | No. | TI Dose | | Reference | Contact time |
|---|---|---|---|---|---|
| group | Replicates | (μg/mL) | | Item Dose | (hours) |
| Negative Control | 3 | 0 | | Water | 24 |
| Positive Control | 3 | N/A | | 1:33 | 24 |
| (Reference Item) | | | | Hydrogen Peroxide | |
| LP34058 (1) | 3 | 17 | (5 AC) | 17 μg/ml | 24 |
| LP34058 (2) | 3 | 67 | (20 AC) | 67 μg/ml | 24 |
| LP34058 (3) | 3 | 333 | (100 AC) | 33 3μg/ml | 24 |

### Organism Origin

### Eimeria

The Eimeria culture was isolated from partridge intestines to provide a source of fresh unsporulated oocysts prior to the start of the in vitro phase of the study.

Cryptosporidium parvum

Live, viable C. parvum oocysts were purchased from Waterborne Inc, USA and shipped prior to the start of the in vitro study. The strain was a calf passaged strain.

### Materials

Distilled water was used in all solutions, unless otherwise stated all solutions was stored at 4° C.

Cryptosporidium parvum
1. The required concentrations of LP34058 were prepared.
2. A known concentration of oocysts was incubated at room temperature in a 1.5 ml tube in the presence of LP34058 for 12 and 24 hours. Control samples were oocysts with no LP34058 present (positive control) and a sample treated with a known C. parvum disinfectant (negative control). This was done in triplicate.
3. The oocysts were washed 3 times in Hanks balanced salt solution to remove the test article.
4. Excystation was achieved by incubating the oocysts at 37° C. with 0.5% trypsin in Hanks balanced salt solution for 30 minutes, then washing once in RPMI1640 medium, and incubating in 0.4% bovine bile salts (Sigma) at 37° C. for 45 minutes.
5. The excystation procedure was monitored by phase contrast microscopy, using a haemocytometer, and stopped when visible sporozoites exceeded 80% of the theoretical maximum (4× the original number of oocysts present per sample).
6. 20 μl of sample was taken and examined microscopically, the condition of the oocysts or free sporozoites was observed in the test samples compared to the negative and positive controls.

### LP34058 Dilutions:

### Dilutions for Cryptosporidium Assay

Stock solution=10 mg/mL=10,000 μg/mL
Formula used: (concentration needed/concentration stock solution)*volume needed=volume of stock solution to add
1. Dilution 1-17 μg/mL LP34058 (0.017 mg/mL) (17/10,000)*50=0.085 mL=85 μL stock solution to 49.915 mL water.
2. Dilution 2-67 μg/mL LP34058 (0.067 mg/mL) (67/10,000)*50=0.335 mL=335 μL stock solution to 49.665 mL water.
3. Dilution 3-333 μg/mL LP34058 (0.333 mg/mL) (333/10,000)*50=1.665 mL=1,665 μL stock solution to 48.335 mL water.

### Dilutions for Eimeria Assay

Final assay volume was 5 mL with a 50:50 split of Eimeria suspension and LP34058 dilution. Therefore LP34058 dilutions had to be twice the stated concentration.
1. Dilution 1-17 μg/mL LP34058 (0.017 mg/mL)*2=34 μg/mL (34/10,000)*10=0.034 mL=34 μL stock solution to 9.966 mL water.
2. Dilution 2-67 μg/mL LP34058 (0.067 mg/mL)*2=134 μg/mL (134/10,000)*10=0.134 mL=134 μL stock solution to 9.866 mL water.
3. Dilution 3-333 μg/mL LP34058 (0.333 mg/mL)*2=666 μg/mL (666/10,000)*10=0.666 mL=666 μL stock solution to 9.334 mL water.

### Organism Assay

The oocysts for each organism were exposed to the 5 treatment options, negative control, positive control, LP34058 (1) (17 μg/mL (5 μg AC/mL)), LP34058 (2) (67 μg/mL (20 μg AC/mL)) and LP34058 (3) (333 μg/mL (100 μg AC/mL)). Three replicates of each were conducted with a 24-hour contact time. Test assays were compared to the positive and negative controls.

### Eimeria

Counts were recorded as the percentage of sporulated vs unsporulated in the first 100 oocysts. Sporulated were counted as containing 4 sporozoites, any abnormalities in appearance noted. These counts inform the claim for biocidal effects of LP34058.

Cryptosporidium parvum

Counts were recorded as the percentage of visible free sporozoites vs oocysts in the first 100 oocysts, (4 free sporozoites equal to one oocyst). Any abnormalities were recorded. These counts inform the claim for biocidal effects of LP34058.

### Reporting Assay Results

The number of intact/viable oocysts/cysts were counted, and the percentage estimated by counting a total of 100 oocysts/cysts. In addition to these observations any abnormalities in terms of size, appearance and damage were observed and recorded where necessary. The oocysts/cysts were slightly flattened under the pressure of a cover slip. Results were obtained via light and/or phase contrast microscopy.

### Statistical Analysis

Data was presented as the mean±S.E.M. A t-test or one-way ANOVA followed by Tukey's test to detect significance among treatment groups. P<0.05 considered to be statistically significant. Results

### Eimeria Species

The results showed that the mean number of oocysts sporulated after 120 hours incubation at 28° C. in the negative control samples was 29.3%. No oocysts were observed to have sporulated in the positive control samples. The mean percentage of sporulated oocysts in all the LP34058 dilutions were not significantly different to the number in the negative control.

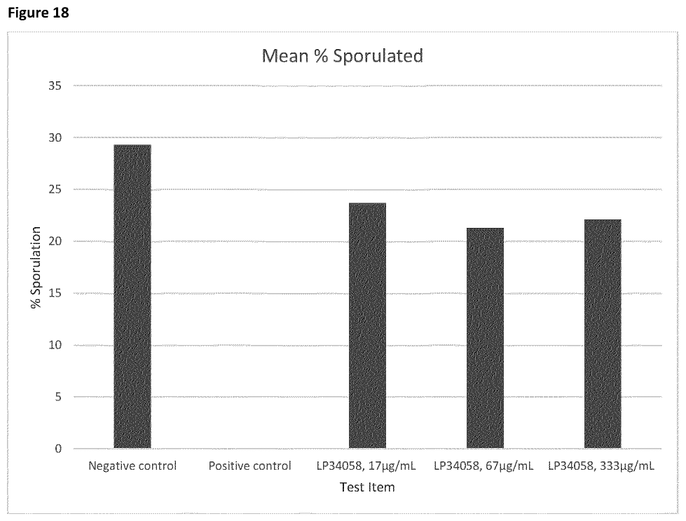

TABLE 16-1
Showing the mean percentage of sporulated
Eimeria oocysts counted, also see FIG. 18.

| | Mean % | |
|---|---|---|
| | Sporulated | SD |
| Negative control | 29.3 | 3.5 |
| Positive control | 0 | 0 |
| LP34058, 17 μg/ml | 23.7 | 4.9 |
| LP34058, 67 μg/ml | 21.3 | 1.5 |
| LP34058, 333 μg/ml | 22.1 | 5.4 |

Cryptosporidium parvum

The number of cysts that successfully excysted in the negative control was 87.9%, the results below show that the positive control, and all 3 dilutions of LP34058 inhibited excystation. In the positive control a mean of 31.7% of cysts excysted, and in the 67 μg/ml LP34058 dilution a mean of only 9.8% of cysts excysted.

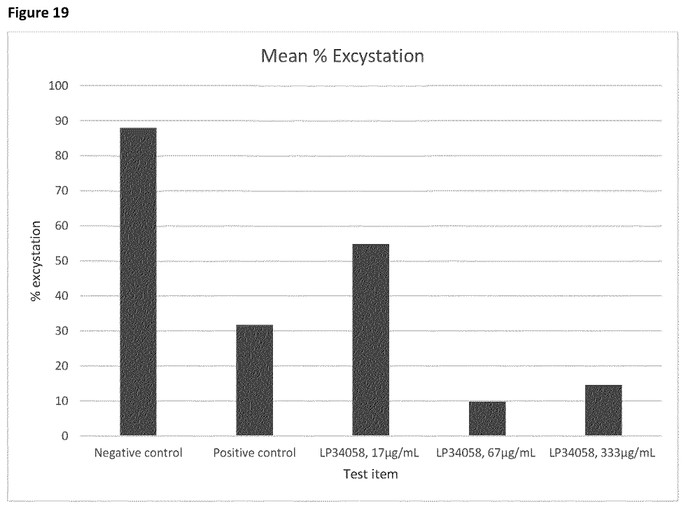

TABLE 16-2
Showing the mean percentage of excysted
Cryptosporidium parvum oocysts, also see FIG. 19.

| | Mean % | |
|---|---|---|
| | Excysted | SD |
| Negative control | 87.9 | 0.3 |
| Positive control | 31.7 | 26.9 |
| LP34058, 17 μg/ml | 54.8 | 22.5 |
| LP34058, 67 μg/ml | 9.8 | 8.5 |
| LP34058, 333 μg/ml | 14.6 | 8.8 |

### Conclusion

The results showed that LP34058 had no significant biocidal effects on Eimeria oocysts. The results for the 3 dilutions were similar (plateaued) indicating that increasing the concentration of the LP34058 would not give a different result.

All 3 dilutions of LP34058 inhibited excystation of the Cryptosporidium parvum oocysts. The 67 μg/ml LP34058 dilution showed the greatest inhibition of excystation, 9.8% of oocysts excysted compared to 87.9% in the negative control.

### Example 17—In Vitro Testing of the Lipopeptide Surfactant Effect on Oomycetes/Fungi

Lipopeptide biosurfactant of DSMZ 34058 (hereinafter referred to as LP34058) obtained in example 3 was tested for effectiveness in inhibiting the growth of some plant pathogenic fungi and plant and animal pathogenic oomycetes. A simple plate assay was performed, whereby LP34058 was added in liquid culture medium resulting in different concentrations of 0, 17, 33, 67, 167, 333 and 665 μg/ml medium. The growth of the fungal or oomycete culture were assessed over time. The following oomycetes/fungi cultures were used in these growth assays (Table 17-1).

TABLE 17-1
List of test strains to be used in this test experiment

| Strains | Classification |
|---|---|
| Aphanomyces astaci | oomycete |
| Aphanomyces sp. | oomycete |
| Saprolegnia parasitica | oomycete |
| Pythium catenulatum | oomycete |
| Pythium dissotocum | oomycete |
| Phytophthora palmivora | oomycete |
| Phytophthora ramorum | oomycete |
| Phytophthora cryptogea | oomycete |
| Fusarium oxysporum sp. gladioli | fungus |
| Fusarium oxysporum f.sp. lycopersici | fungus |
| Verticillium dahliae | fungus |

Same medium (24 g/L Potato Dextrose medium (PD) in small Petri dishes (5.5 cm diameter)) was used for all strains to be able to compare results between different strains.

LP34058 stock solution was prepared freshly for each test by dissolving LP34058 in water with addition of 0.2M NaOH (sodium hydroxide). The LP34058 solution was added to PD to provide the final concentrations of 0, 17, 33, 67, 167, 333, and 665 μg/ml.

The isolates were grown on PD-agar plates at 24° C. This provided sufficient material for the growth and LP34058 inhibition experiments.

An agar plug (1 cm diameter cut with a cork borer) of freshly grown mycelia on PDA plates was transferred to 5 ml liquid PD (containing various amounts of LP34058) in 5.5 cm Petri dishes. The plates were incubated at 12° C. for 2 weeks.

Images of the cultures were taken on day 4, 7, 9 and 11 after transfer to the liquid medium with and without LP34058.

### Results

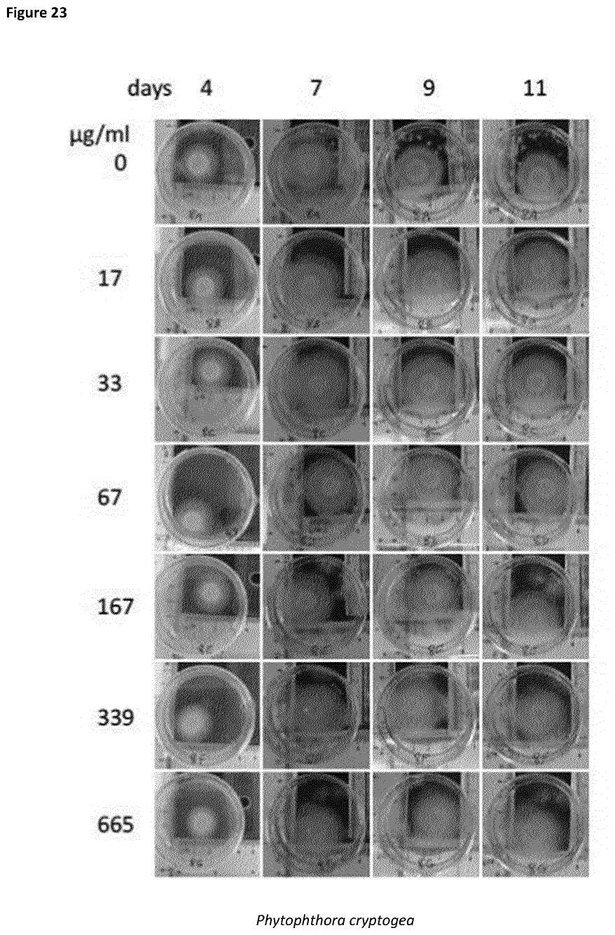

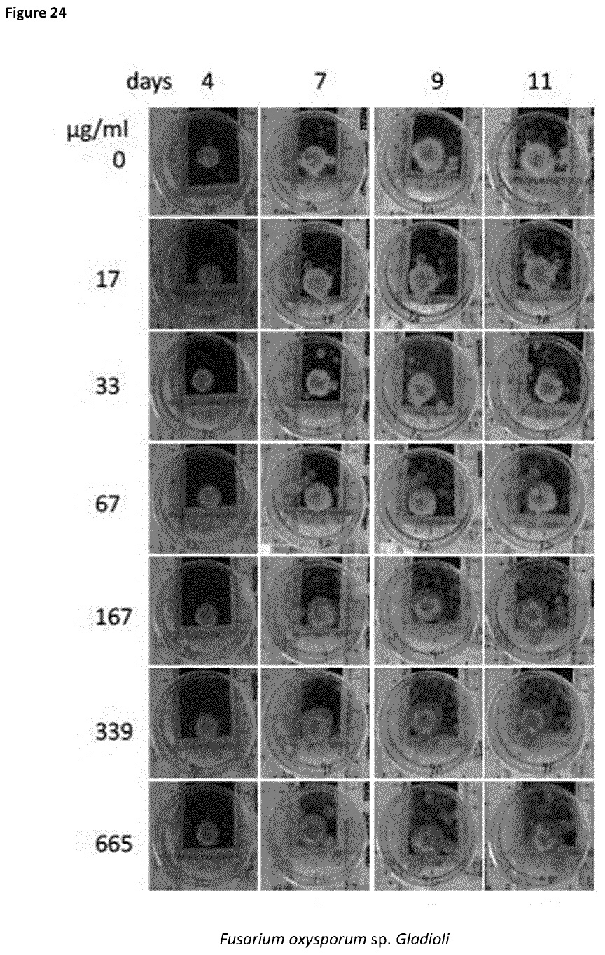

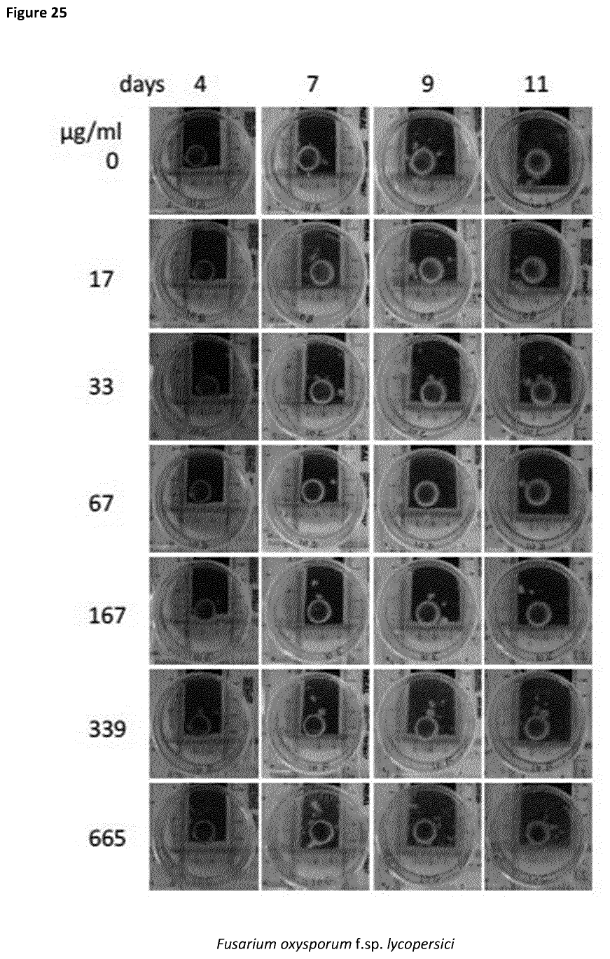

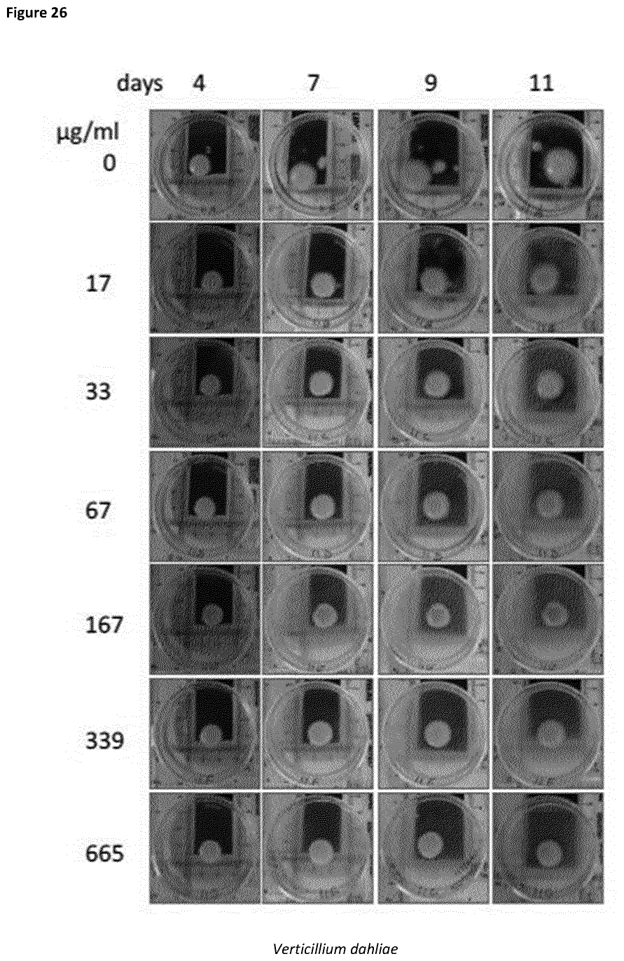

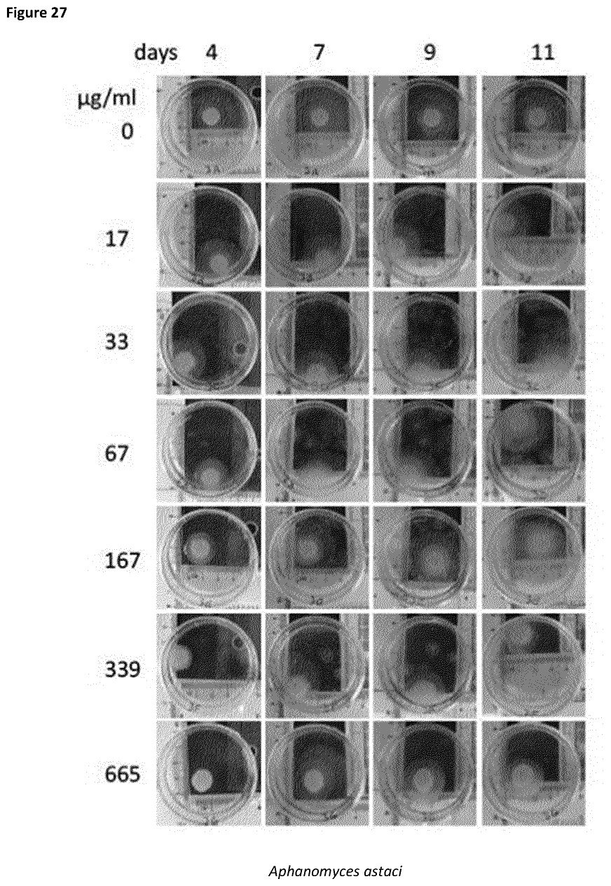

The plant pathogenic oomycetes Phytophthora ramorum (FIG. 22), Phytophthora cryptogea (FIG. 23), and the fungal plant pathogens Fusarium oxysporum sp. gladioli (FIG. 24), Fusarium oxysporum f.sp. lycopersici (FIG. 25), and Verticillium dahlia (FIG. 26) were hardly or not affected by LP34058.

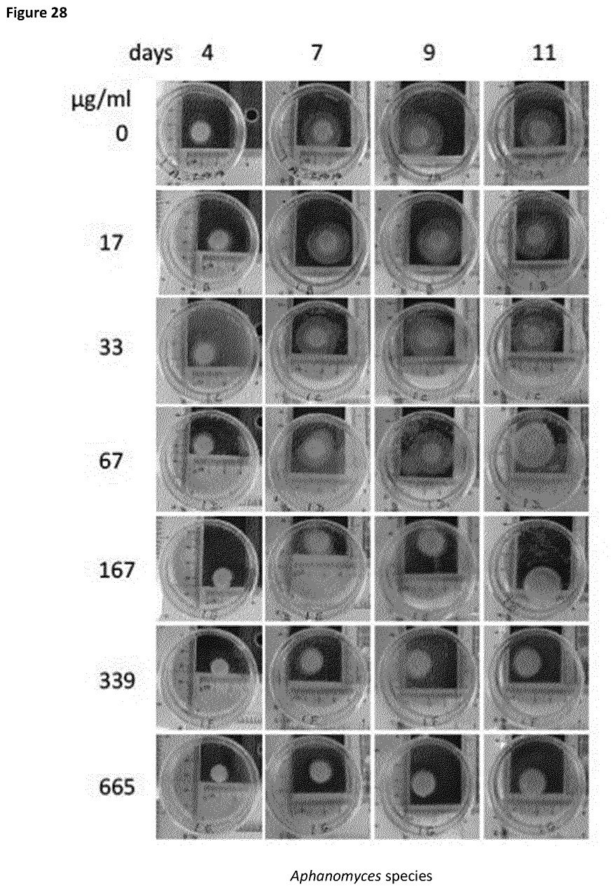

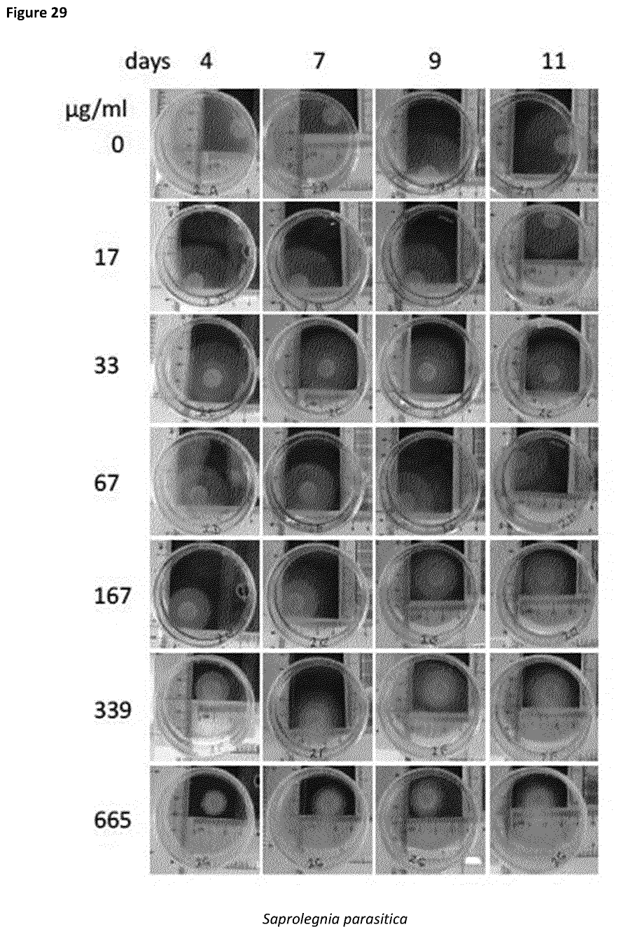

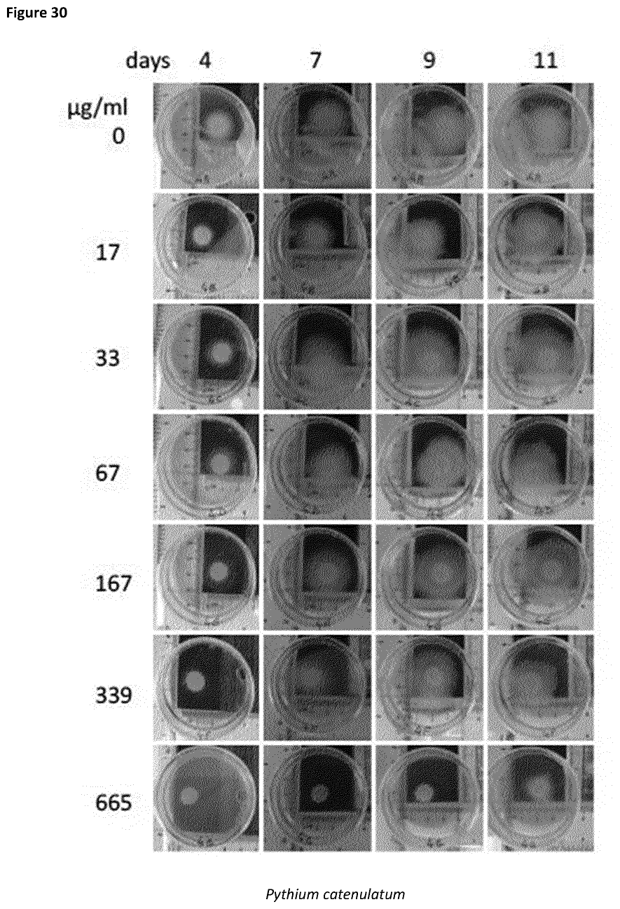

The isolates showing some reduction in growth, especially at higher concentrations of LP34058, were the animal pathogenic oomycetes including Aphanomyces astaci (FIG. 27), an unknown Aphanomyces species (FIG. 28), and Saprolegnia parasitica (FIG. 29). Concentrations of LP34058 at 339 and 667 μg/ml resulted in growth inhibition of A. astaci. Also S. parasitica showed clear inhibition at the highest concentration that was tested (667 μg/ml).

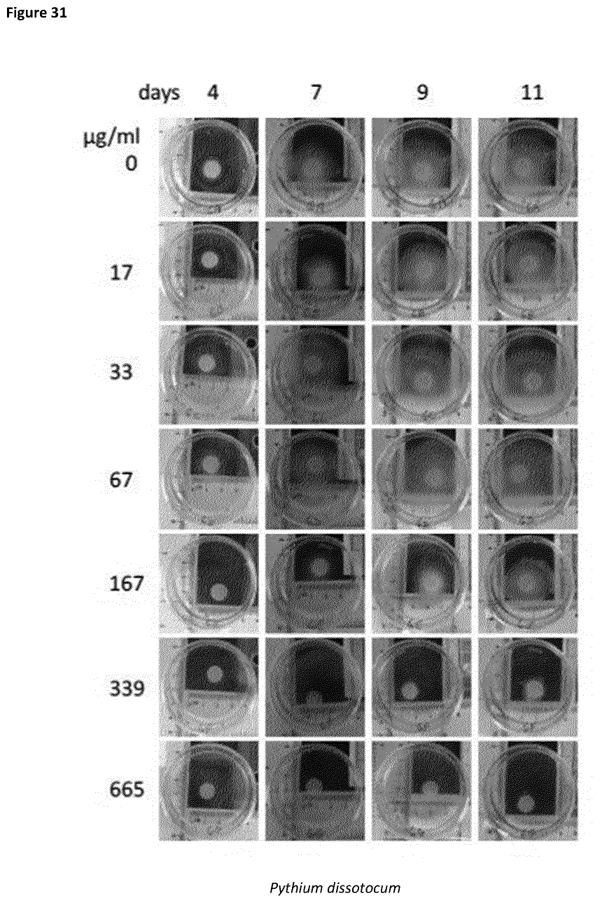

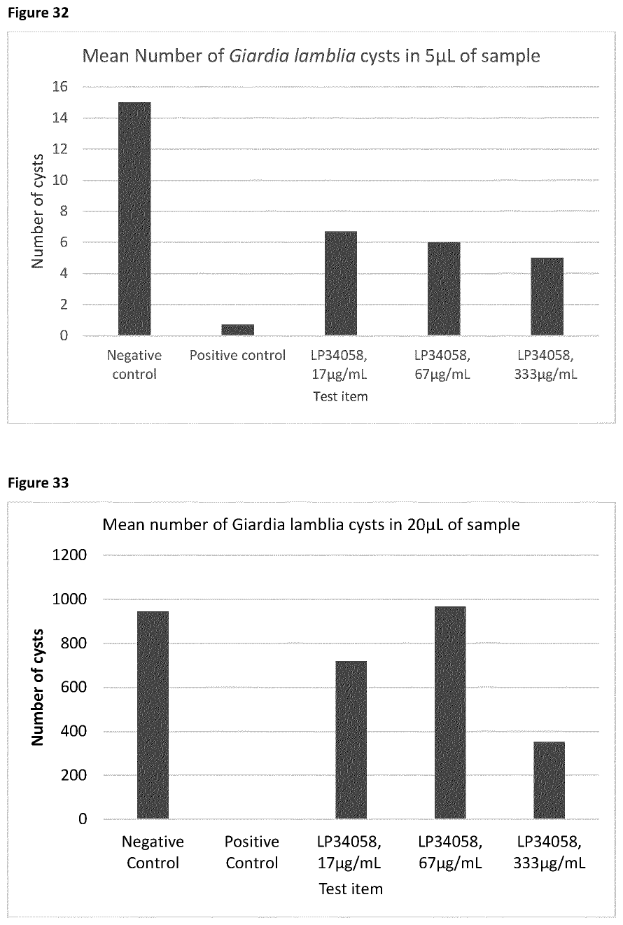

In addition, the plant pathogenic Pythium species showed a very clear reduction in growth especially with high concentrations of LP34058. Pythium catenulatum (FIG. 30) had very strong growth reduction visible at the highest concentration, especially up to day 9 after transfer. Pythium dissotocum (FIG. 31) was the most affected isolate tested. It essentially stopped growing at concentrations higher than 339 μg/ml.

The inhibition experiments demonstrated that LP34058, at high concentrations, has a modest to good effect against the growth of the animal pathogenic oomycetes Aphanomyces astaci and Saprolegnia parasitica, as well as an unknown Aphanomyces species isolated from an aquarium fish shop. The plant pathogenic fungi and Phytophthora species seemed relatively unaffected by LP34058, whereas the tested plant pathogenic Pythium species (i.e. P. dissotocum and P. catenulatum) were considerably inhibited at high concentrations of LP34058. P. dissotocum was the most sensitive isolate, where inhibition of growth was seen at concentrations of 167 ag/ml and higher.

### Example 18 In Vitro Testing of Lipopeptide Surfactant on Ciliates/Tetrahymena Spp

In this study, a lipopeptide biosurfactant of DSMZ 34058 (hereinafter referred to as LP34058) obtained in example 3 was used to test the effects on survival of two strains of Tetrahymena (T. thermophila and T. pyriformis) using in vitro bioassays. The T. thermophila B2086 II (Resource Identification Citation: TSC_SD00709) and T. pyriformis GL-C(Resource Identification Citation: TSC_SD00707) strain used in the bioassays was purchased from the Tetrahymena Stock Center at Cornell University.

A single parent culture of each strain was grown to mid/late exponential phase in a 10 mL Modified Neff media (Cassidy-Hanley 2012) with penicillin and streptomycin (250 ag/ml) in a T25 cell flask incubated at 30° C. From the surface of the parent culture, 1 ml was collected and diluted to an appropriate cell density. After the dilution, 5 μL was pipetted to each well of a 96-well microtiter plate and the number of Tetrahymena cells for each well was determined just prior to the test using a microscope. Only individual wells containing 5-15 cells was utilized for the assay.

A stock solution of 10 mg/ml LP34058 was prepared utilizing Modified Neff media as a diluent, immediately prior to assay start. Stock solution was diluted to twice the effective concentrations listed in Table 18-1. For each LP34058 treatment and Modified Neff media alone (i.e., negative control), 5 μL was pipetted into each of four wells containing a pre-recorded number of T. thermophila. Cell survival was assessed and recorded by direct counts of cell abundance under microscope at 0, 15, 30, 45 and 60 min after addition of LP34058 solution or at 0 and 60 min after addition of Modified Neff media in the negative control treatment. T. thermophila survival was determined by assessing mobility and ciliary movement, with non-motile and lysed cells considered as dead. The 96-well microtiter plate was incubated at room temperature between observations.

TABLE 18-1

| | LP34058 treatments |
|---|---|
| | (mg active |
| Treatment | compound/L) |
| 1 | 6.3 |
| 2 | 12.6 |
| 3 | 18.9 |
| 4 | 25.2 |
| 5 | 31.5 |
| 6 | 37.8 |

### Results

Tetrahymena thermophila

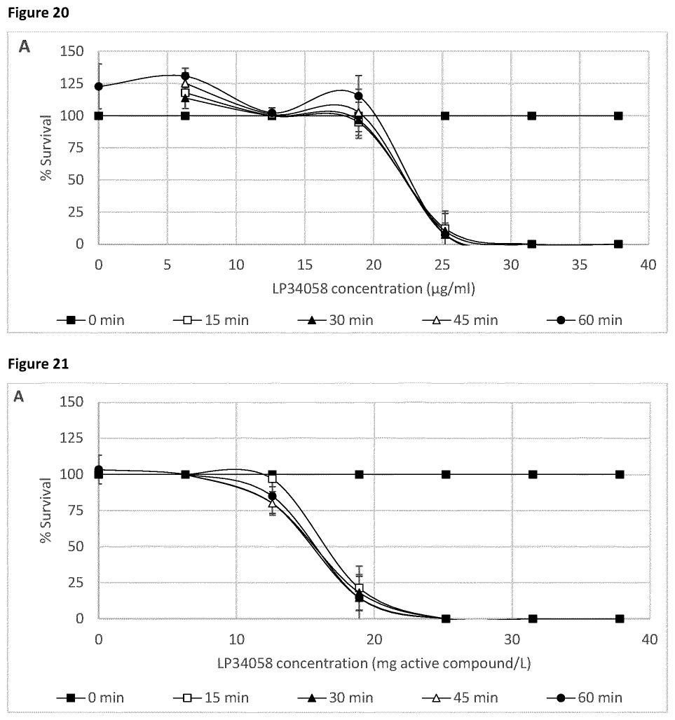

LP34058 concentrations of >31.5 mg active compound/L immediately killed all T. thermophila and no surviving cells were observed after 0 min. Cells in these high concentrations were almost all lysed with no intact T. thermophila visible after 60 min (see FIG. 20). Exposure to 25.2 mg active compound/L resulted in 12% and 10.2% survival after 15 and 30 min, respectively, whereafter survival was stable at 7.4% until the end of the test. Concentrations of 6.3, 12.6 and 18.9 mg active compound/L did not result in any mortality as survival in these treatments were between 95-130% and a general increase in the number of cells were seen for these lower LP34058 treatments. The number of cells in the negative control (0 mg/L) had also increased after 60 min to 122%, showing the same tendencies as the three lowest LP34058 treatments.

TABLE 18-2
Observed average count ± standard deviation of motile Tetrahymena thermophila
in quadruplicate wells for effective concentrations of LP34058 and
negative control at each observation time point.

| | Average number of Tetrahymena thermophila | | | | | | |
|---|---|---|---|---|---|---|---|
| Observation | 0 mg | 6.3 mg | 12.6 mg | 18.9 mg | 25.2 mg | 31.5 mg | 37.8 mg |
| Time (Min) | AC/L | AC/L | AC/L | AC/L | AC/L | AC/L | AC/L |
| 0 | 7.7 ± 2.5 | 8.5 ± 2.1 | 9.3 ± 1.9 | 10.4 ± 2.2 | 9.9 ± 3.2 | 11.3 ± 3.2 | 8.4 ± 2.2 |
| 15 | NA | 10.0 ± 2.4 | 9.3 ± 1.9 | 10.9 ± 2.8 | 1.3 ± 1.3 | 0 ± 0 | 0 ± 0 |
| 30 | NA | 9.6 ± 2.4 | 9.3 ± 1.9 | 10.3 ± 3.3 | 1.0 ± 1.2 | 0 ± 0 | 0 ± 0 |
| 45 | NA | 10.6 ± 2.7 | 9.4 ± 2.0 | 11.0 ± 4.0 | 0.8 ± 0.8 | 0 ± 0 | 0 ± 0 |
| 60 | 9.3 ± 2.6 | 11.3 ± 3.1 | 9.5 ± 2.2 | 12.1 ± 3.7 | 0.8 ± 0.8 | 0 ± 0 | 0 ± 0 |

The LD50 value for mortality after 60 min was estimated to be 22.3 mg active compound/L using the AAT Bioquest online calculator.

Tetrahymena pyriformis

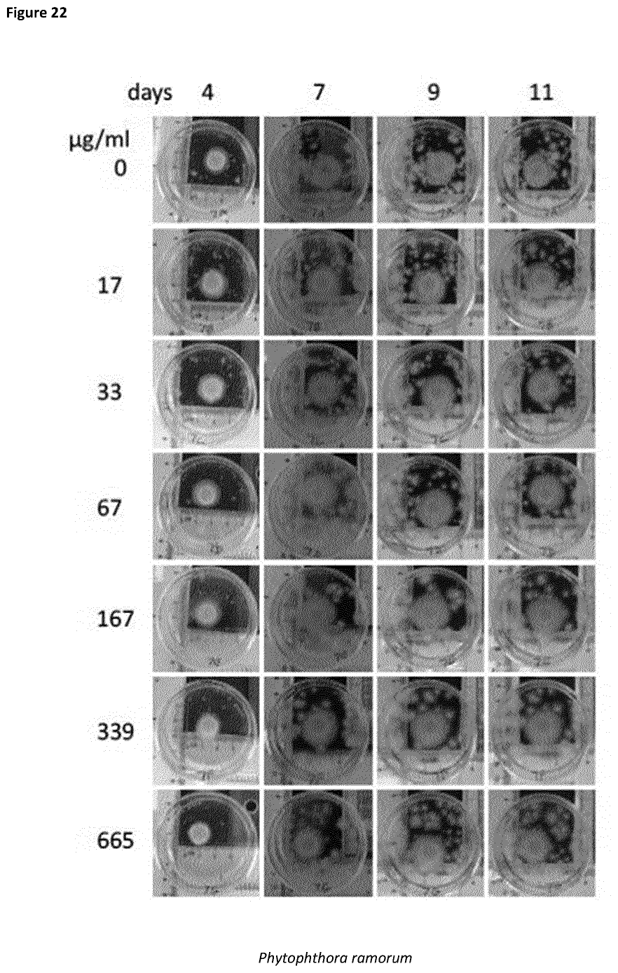

T. pyriformis exposed to >25.2 mg active compound/L was all lysed and killed after 15 min with cells exposed to 18.9 mg active compound/L instantly exhibiting high mortality, see FIG. 21. LP34058 concentration of 12.6 mg active compound/L resulted in minor mortality (i.e., 15-20%) after 30 min while the lowest concentration at 6.3 mg active compound/L did not lead to any mortality, similar to the negative control.

TABLE 18-3
Observed average count ± standard deviation of motile Tetrahymena pyriformis
in quadruplicate wells for effective concentrations of LP34058 and
negative control at each observation time point.

| | Average number of Tetrahymena pyriformis | | | | | | |
|---|---|---|---|---|---|---|---|
| Observation | 0 mg | 6.3 | AC/L | AC/L | AC/L | AC/L | AC/L |
| Time (Min) | AC/L | AC/L mg | 12.6 mg | 18.9 mg | 25.2 mg | 31.5 mg | 37.8 mg |
| 0 | 5.4 ± 2.9 | 10.3 ± 3.8 | 6.5 ± 1.1 | 7.0 ± 0.7 | 5.3 ± 0.4 | 6.3 ± 1.1 | 5.5 ± 0.5 |
| 15 | NA | 10.3 ± 3.8 | 6.3 ± 0.8 | 1.5 ± 1.1 | 0.0 ± 0.0 | 0.0 ± 0.0 | 0.0 ± 0.0 |
| 30 | NA | 10.3 ± 3.8 | 5.3 ± 1.3 | 1.3 ± 0.8 | 0.0 ± 0.0 | 0.0 ± 0.0 | 0.0 ± 0.0 |
| 45 | NA | 10.3 ± 3.8 | 5.3 ± 1.3 | 1.0 ± 1.0 | 0.0 ± 0.0 | 0.0 ± 0.0 | 0.0 ± 0.0 |
| 60 | 5.5 ± 2.8 | 10.3 ± 3.8 | 5.5 ± 1.1 | 1.0 ± 1.0 | 0.0 ± 0.0 | 0.0 ± 0.0 | 0.0 ± 0.0 |

The LD50 value for mortality after 60 min was estimated to be 15.3 mg active compound/L using the AAT Bioquest online calculator.

### Conclusion

LP34058 was instantly lethal to both Tetrahymena strain at concentrations above 31.5 mg active compound/L with no or very low survival observed at 25.2 mg active compound/L after 60 min. The T. pyriformis strain was found to be less tolerant to LP34058 than T. thermophila as 18.9 mg active compound/L resulted in almost 100% mortality in the former but showed no killing effect on the latter. This was also apparent in the lower LD50 estimated for T. pyriformis (15.3 mg active compound/L) compared to T. thermophila (22.3 mg active compound/L).

### Example 19—In Vitro Testing of the Lipopeptide Surfactant Effect on Flagellated Protists

Lipopeptide biosurfactant of DSMZ 34058 (hereinafter referred to as LP34058) obtained in example 3 was tested for biocidal effect on Giardia lamblia cysts as a disinfectant.

### Study Design

The negative control group did not contain LP34058 and enabled observation of the chosen organism without the test item or any parasiticide being present. A reference item was used which is known to kill the intended pathogen at the manufacturers recommended dosage.

TABLE 19-1
Giardia lamblia overview

| | | | | Reference | Contact |
|---|---|---|---|---|---|
| Treatment | No. | TI Dose | | Item | time |
| group | Replicates | (μg/mL) | | Dose | (hours) |
| Negative Control | 3 | 0 | | | 24 |
| Positive Control | 3 | N/A | | 1:32 Sodium | 24 |
| (Reference Item) | | | | hypochlorite | |
| | | | | solution | |
| LP34058 (1) | 3 | 17 | (5 AC) | | 24 |
| LP34058 (2) | 3 | 67 | (20 AC) | | 24 |
| LP34058 (3) | 3 | 333 | (100 AC) | | 24 |

AC: Concentration of active in the formulation

### Organism Origin

Live, viable G. lamblia cysts were purchased from Waterborne Inc, USA and shipped prior to the start of the in vitro study.

### Organism Assay

Giardia lamblia cysts were exposed to the 5 treatment options, negative control, positive control, LP34058 (1) (17 μg/mL (5 μg AC/mL)), LP34058 (2) (67 μg/mL (20 μg AC/mL)) and LP34058 (3) (333 μg/mL (100 μg AC/mL)). Three replicates of each were conducted with a 24-hour contact time. Test assays were compared to the positive and negative controls.

Counts were recorded as the number of cysts present in 5 μL of sample. No excystation of the cysts was observed, therefore the percentage of excyzoites vs encysted cysts in the first 100 cysts could not be recorded, however lysing of cysts was observed. These counts inform the claim for biocidal effects of LP34058.

Fresh parasites were sourced, and the assay repeated, counts were recorded as the number of cysts present in 20 μL of sample (a larger aliquot of sample was examined than in the previous testing to better assess the number of cysts remaining at the end of the assay).

### Materials

Distilled water was used in all solutions, unless otherwise stated all solutions was stored at 4° C. Bovine serum was heat-inactivated by placing at 56° C. for 30 minutes. Peptone digest, yeast extract, and bovine serum were lot tested.

### 6.5% Bile Solution

6.5 g Bovine and Ovine Bile (Sigma Aldrich)
100 mL Water
Filter sterilize with a 0.45 μM filter unit
Store at 4° C. for up to 2 months

### 2.28% Ferric Ammonium Citrate

2.28 g Ammonium iron (III) Citrate (Sigma Aldrich)
100 μL Water
Filter sterilize with a 0.45 μM filter unit
Store at 4° C. for up to 2 months

### Giardia Media

Giardia Media (Complete modified TYI-S-33 Medium (Sigma Aldrich)

### Solution 1:

10 g Peptone from milk solids (Sigma Aldrich)
5 g D-glucose
5 g Bacto Yeast Extract (Gibco)
1 g Sodium Chloride
1.05 g Sodium Bicarbonate
400 μl Water
4 mL Sterile 6.5% Bile Solution
1.5 mL Sterile 2.28% Ferric Ammonium Citrate

Alternatively, Sodium Bicarbonate can be substituted for 0.3 g of 3% KH₂PO₄ and 0.65 g K₂HPO₄·3H₂0. As media components for Giardia media solution 1 solubilise and combine, media should turn from opaque into a clear solution.

### Solution 2:

1 g L-cysteine Hydrochloride (Sigma Aldrich)
0.05 g Ascorbic Acid
30 mL Water
Adjust pH of solution 2 to 7.0-7.2 with 10N Sodium Hydroxide
Add solution 2 to solution 1 and mix for at least 20-30 minutes
Add 50 mL Adult Bovine Serum and 5 mL Antibiotic-Antimycotic (Gibco)
Sterile filter with 0.45 μm filter and aliquot in BSC (if needed)
Store up to 1-2 weeks at 4° C. Over time the cysteine in the media will oxidize to cystine and precipitate.
Media should be discarded at this time and fresh media remade.

### Excystation Stage 1 Solution

68 mg L-Cysteine (non HCL) (Sigma Aldrich, cat. no. C7352)
68 mg L-Glutathione Reduced (Sigma Aldrich, cat. no. G4251)
52 mg Sodium Bicarbonate
7 mL 1× Hank's Balanced Salt Solution
18 mL Water
Adjust pH to 2.0 and sterilize with 0.45 μm filter
This media must be made fresh and used immediately on the day of preparation.

### Excystation Stage 2 Solution

100 mg Trypsin Type II-S
10 mL 1× Tyrode's Salt Solution, pH=8
Mix and sterilize with 0.45 μm filter

This media must be made fresh and used immediately on day of preparation.

### Reporting Assay Results

The number of intact/viable cysts were counted, and the percentage estimated in a count of 100 cysts. In addition to these observations any abnormalities in terms of size, appearance and damage were observed and recorded where necessary. It should be noted that the cysts were slightly flattened because of being under the pressure of a cover slip and not because of treatment. Results were obtained via light and/or phase contrast microscopy.

### Statistical Analysis

Data was presented as the mean±S.E.M. A t-test or one-way ANOVA followed by Tukey's test to detect significance among treatment groups. P<0.05 is considered to be statistically significant.

### Results

Excystation of the G. lamblia cysts did not occur in any of the samples. Results shown in table 2 indicate that the number of cysts present in a 5 μL aliquot were significantly decreased in the positive control compared to the negative control. There was a positive dose-response lysis with increasing concentration of LP34058, though none of the dilutions appeared as effective as the positive control.

TABLE 19-2
Statistical Summary and Analysis for G. lamblia

| | | | p-values # | | | | |
|---|---|---|---|---|---|---|---|
| | Cysts | | Negative | Positive | 17 μg/mL | 67 μg/mL | 333 μg/L |
| Test item | Mean | SD | control | control | LP34058 | LP34058 | LP34058 |
| Negative control | 15 | 5.3 | • | 0.005 | 0.322 | 0.326 | 0.156 |
| Positive control | 0.7 | 1.2 | • | • | 0.109 | 0.108 | 0.234 |
| 17 μg/mL LP34058 | 6.7 | 5.7 | • | • | • | 1 | 0.983 |
| 67 μg/mL LP34058 | 6 | 2 | • | • | • | • | 0.982 |
| 333 μg/mL LP34058 | 5 | 4.6 | • | • | • | • | • |

No analysis of % excyzoites performed as all observations were zero
p-values based on Tukeys multiple comparison test on square root transformed cyst counts

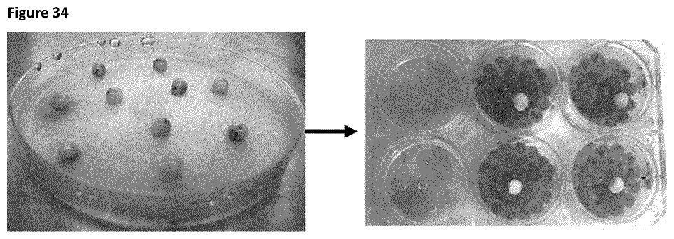

The mean number of G. lamblia cysts counted in 5 μl of sample is shown in FIG. 32.

Repeat Testing of G. lamblia

The number of G. lamblia cysts in the positive control were significantly reduced by 99.7% in the 20 μL of sample examined, compared to the negative control. The number of cysts lysed in the 333 μg/mL LP34058 solution was decreased compared to the negative control (although not significantly). Results for all dilutions of LP34058 were significantly different to the positive control. No excystment occurred in any to the test samples.

TABLE 19-3
Statistical Summary and Analysis for G. lamblia in 20ul of sample.

| | | | p-values # | | | | |
|---|---|---|---|---|---|---|---|
| | Cysts | | Negative | Positive | LP34058, | LP34058, | LP34058, |
| Test Item | Mean | SD | control | control | 17 μg/mL | 67 μg/ml | 333 μg/mL |
| Negative control | 945.3 | 357.7 | • | 0.001 | 0.916 | 1 | 0.197 |
| Positive control | 2.7 | 2.5 | • | • | 0.004 | 0.001 | 0.036 |
| LP34058, 17 μg/mL | 719 | 356.5 | • | • | • | 0.94 | 0.554 |
| LP34058, 67 μg/mL | 966.3 | 652.8 | • | • | • | • | 0.222 |
| LP34058, 333 μg/mL | 350.7 | 100.8 | • | • | • | • | • |

No analysis of % excyzoites performed as all observations were zero
p-values based on Tukeys multiple comparison test on square root transformed cyst counts

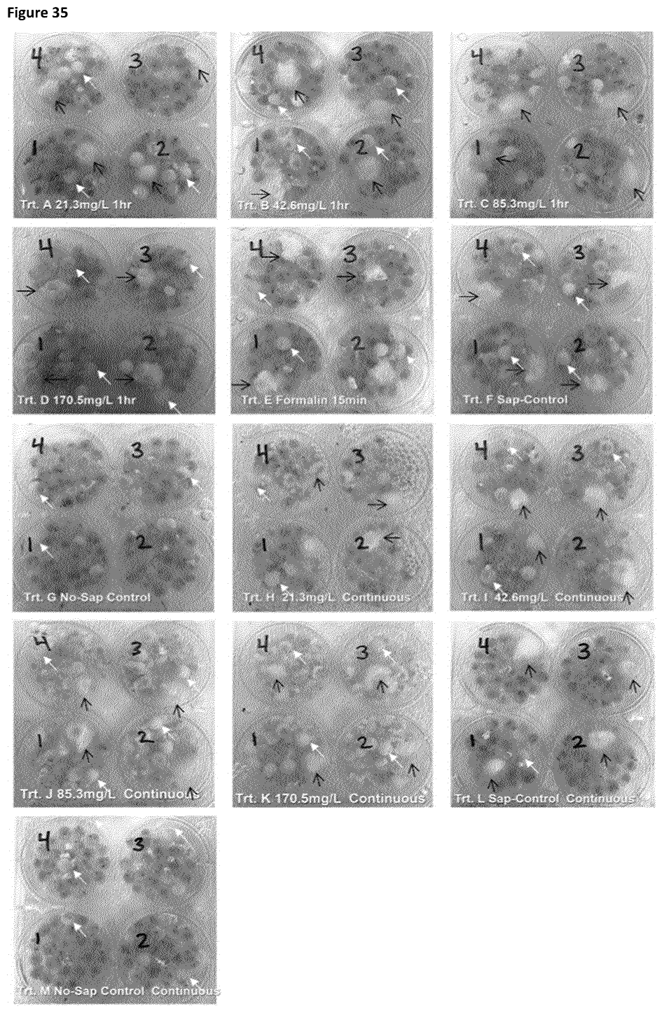

The mean number of G. lamblia cysts counted in 20 μL of sample is shown in FIG. 33.

### Conclusions

The G. lamblia assay showed a positive lysis dose-response with increasing concentration of LP34058, though none of the dilutions appeared as effective as the positive control. No excystment occurred in the negative control samples.

The repeat G. lamblia assay results were consistent with the first assay, no excystment occurred in the negative control, but lysis was seen in both positive control and LP34058 assays.

This pilot study suggests that LP34058 could have a role to play in control of environmental or water contamination with G. lamblia.

### Example 20—In Vitro Testing of the Lipopeptide Surfactant Effect on Viruses

Lipopeptide biosurfactant of DSMZ 34058 (hereinafter referred to as LP34058) obtained in example 3 was tested for effectiveness against the enveloped virus causing Viral hemorrhagic septicemia (VHS).

### Materials and Methods

Different concentrations of LP34058 were incubated in a serial dilution of VHS virus. A stock solution of the LP34058 was prepared by dissolving 1.9 mg of LP34058 in 600 μl RNase/DNase free water plus 18.75 μl of 0.2M NaOH followed by extensive vortexing and centrifuge (14000 rpm) for 5 min. To prepare different testing concentrations of LP34058 (320 μg/ml, 160 μg/ml, 80 μg/ml, 40 μg/ml, 20 μg/ml, and 10 μg/ml), 168.4 μl of stock solution was diluted in 1 ml transport media (320 μg/ml) and other concentrations were made by adding 250 μl of the previous concentration to 500 μl transport media to reach to the lowest concentration. Earlier studies on the virus titration showed that the available viral stock loose strength against the host cell on 10⁻⁶ dilution, therefore, viral dilutions until 10⁻⁵ were selected. Serial dilutions of the virus (10⁻¹,10⁻²,10⁻³,10⁻⁴,10⁻⁵) were prepared in a 96 well plate and incubated with LP34058 at 15° C. for 3 h. To investigate the potential effect of NaOH on the virus, the virus was incubated with NaOH at the same plate for the same period. After 3 h, the mixture of virus and LP34058 was inoculated to two 96 wells plates coated with EPC (epithelioma papulosum cyprini) cells for 24 h. Inoculated plates were placed at 15° C. for 72 h.

### Results:

Plates were assessed for both CPE (cytopathic effect) and GFP (green fluorescent protein) for each well, it means any damage in cells compared to control cells and also the presence of virus characterized by GFP was evaluated. For LP34058 concentrations between 80 and 10 μg/ml, clear CPE and GFP was observed meaning that LP34058 could not deactivate the virus at this range of concentrations. For concentrations 320 and 160 μg/ml (in particular 160 μg/ml) GFP was only detected in a few wells, and CPE was positive but weak. This might indicate a toxic effect of high concentrations of LP34058 on VHS virus. Damage on cells with LP34058 only (320 and 160 μg/ml) and NaOH only was observed in negative controls.

### Conclusion:

High concentrations of LP34058 (320 and 160 μg/ml) might damage or deactivate the VHS virus. However, this effect might be due to the toxic effects of LP34058 on cells.

### Example 21—In Vivo Testing of the Lipopeptide Surfactant Effect on the Subject Infected with a Flagellated Protist

Lipopeptide biosurfactant of DSMZ 34058 (hereinafter referred to as LP34058) obtained in example 3 was tested for effectiveness against Ichthyobodo (Costia) infecting Asian sea bass (Lates calcarifer).

### Methodology

Twenty Asian sea bass were treated (in duplicate, 10 fish per tank) via immersion administration with a dose of LP34058 (20 mg/L), for 4 hours in tanks with 30 L of fresh water. Fish were held at 24±1° C. and the average weight at the time of treatment was approximately 1 g. To standardize the scoring for the analysis 5 fish were euthanized before the treatment and the Ichthyobodo infestation was evaluated following the instructions in table 21-1.

TABLE 21-1

| | Affected gill filamentous | |
|---|---|---|
| Severity | (one arch with 10 filamentous) | Score |
| Severe | 5-10 filamentous affected (50%) | 3 |
| Moderated | 2-4 filamentous affected (20%-40%) | 2 |
| Mild | 1 filamentous affected (10%) | 1 |
| Absence | 0 filamentous affected | 0 |

The results during standardization showed that 5 out of 5 fish presented severe infection. After confirmation of the presence of the parasite, the bay preparation for treatment was started. 10 morbid fish were placed in the control bucket and 10 morbid fish were placed in the treatment bucket.

The immersion treatment lasted for 4 hours; the treatment tanks were supported with oxygen stones and monitored for any kind of clinical signs.

After 4 hours fish were euthanized, and gill health conditions was assessed by wet mount analysis. The analyst received samples not knowing which bags (5 fish/bag) contained fish from control group or the treatment group (blind analysis). The infection level in each fish was quantified according to table 21-1 and presented in Table 21-2.

TABLE 21-2

| Fish number | Bag 1 | Bag 2 | Bag 3 | Bag 4 |
|---|---|---|---|---|
| 1 | 3 | 0 | 0 | 0 |
| 2 | 3 | 0 | 0 | 0 |
| 3 | 0 | 0 | 0 | 0 |
| 4 | 0 | 0 | 0 | 0 |
| 5 | 1 | 0 | 0 | 0 |

Bags 1 and 3 contained control fish
Bags 2 and 4 fish treated with the Test Product (LP34058)

### Results

In the control group, 3 out of 10 fish presented Ichthyobodo infection (30% prevalence). The p value (t-test) was 0.093918. In the group exposed to LP34058 no Ichthyobodo was found. The control group had an average score of 0.7 vs 0 for the treated group.

Besides the absence of Ichthyobodo in the treated group, some changes in the tissue were observed, cells appeared swollen, however this test is not sensitive enough to evaluate tissue changes.

### Conclusions

The control group showed 50% of parasite prevalence with different severities, while the group treated with LP34058 did not show parasites after treatment.

Wet mount gives an idea of the presence of the parasites, however the changes observed in tissue cannot be evaluated only with wet mounts. Histopathology is recommended to prove the safety of the product regarding gill health.

Even though the fish reacted well to the treatment (no mortalities), It is recommended first to evaluate the impact of the treatment in gill tissue through histopathology in fish without any pathology.

### Example 22—In Vivo Testing of the Lipopeptide Surfactant Effect on the Subject Infected with an Oomycete

Lipopeptide biosurfactant of DSMZ 34058 (hereinafter referred to as LP34058) obtained in example 3 was tested for effectiveness to control saprolegniosis by assessing survival of Atlantic salmon (Salmo solar L.) eggs challenged with Saprolegnia diclina.

### Materials and Methods

### Test Article and Test System

LP34058 was used at four different concentrations to prepare immersion baths using freshwater. 1300 developing eyed Atlantic salmon eggs were used for the study to test the efficacy of LP34058 administration against saprolegniosis.

### Study Design

The study utilized 13 treatment groups each having 100 eyed eggs divided into four replications housed in four wells of 6-well plates. Each 6-well plate was submerged in a plastic container containing 2 L freshwater; treatment group details in Table 23-1. Eggs were challenged with S. diclina by introducing a pre-colonized trojan egg (FIG. 1) in each well except treatment G and M that served as no-Saprolegnia control.

TABLE 22-1
Treatment group information

| | | | Total | | | |
|---|---|---|---|---|---|---|
| Treatment | | # Eggs/ | eggs/ | | Treatment | |
| # | Replication | replication | treatment | Chemical treated | Dose (mg/L) | Mode of treatment |
| A | 4 | 25 | 100 | LP34058 | 21.3 | 1 hr bath daily |
| B | 4 | 25 | 100 | LP34058 | 42.6 | 1 hr bath daily |
| C | 4 | 25 | 100 | LP34058 | 85.3 | 1 hr bath daily |
| D | 4 | 25 | 100 | LP34058 | 170.5 | 1 hr bath daily |
| E | 4 | 25 | 100 | Formalin | 1665 ppm | 15 min bath daily |
| F | 4 | 25 | 100 | Control-Sap | 0 | 1 hr pseudo-bath daily |
| G | 4 | 25 | 100 | Control - No sap | 0 | 1 hr pseudo-bath daily |
| H | 4 | 25 | 100 | LP34058 | 21.3 | Continuous |
| I | 4 | 25 | 100 | LP34058 | 42.6 | Continuous |
| J | 4 | 25 | 100 | LP34058 | 85.3 | Continuous |
| K | 4 | 25 | 100 | LP34058 | 170.5 | Continuous |
| L | 4 | 25 | 100 | Control-Sap | 0 | Continuous |
| M | 4 | 25 | 100 | Control - No sap | 0 | Continuous |

### Trojan Egg Preparation

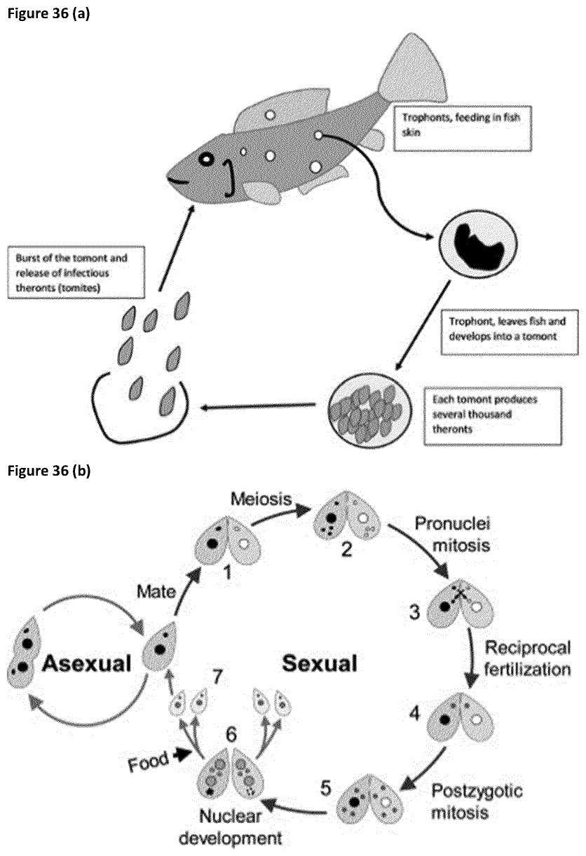

Ca. 50 eyed Atlantic salmon eggs were heat killed at 60° C. for 1 minute in a small beaker with 50 ml freshwater followed by cooling down at 4° C. for 60 mins. Then, the eggs were transferred to S. diclina grown petri dishes using a pair of forceps and incubated at 15° C. for three days to ensure colonization (FIG. 34).

### Egg Disinfection, and Treatment Distribution

Ca. 1,300 Atlantic salmon eyed eggs were treated with 1665 ppm 37% formalin for 15 minutes before distributing into 6-well plates. Only healthy-looking live eggs were recruited in the study. Well plates were carefully submerged into the experimental containers having 2 L freshwater according to Table 1. The containers were stored in an incubator with aeration where temperature was maintained at 4±2° C. throughout the study period.

### LP34058 Stock Solution

According to Table 23-2, 85.25 and 682.03 mg of LP34058 were weighed and mixed in chilled distilled water to prepare stock solutions for 1 hr and continuous immersion baths. To ensure LP34058 is fully dissolved, stock solutions were freshly prepared and stored at 4-6° C. for at least 1 hr prior to immersion baths preparation for the target dose (Table 23-2). Both stock solution and immersion baths were freshly prepared on 1^{st}, 5^{th} and 9^{th} day post challenge during the study. In addition, formalin bath was prepared by mixing 416.25 μL 37% formaldehyde in 250 mL freshwater.

TABLE 22-2
Stock solution and bath preparation for immersion challenge.

| | Stock | Amount | | | | |
|---|---|---|---|---|---|---|
| | solution | LP34058 to | Vol. from Stock | Initial | Final | Final bath |
| Mode of | Vol. | stock solution | solution added | bath vol. | bath vol. | concentration |
| treatment | (ml) | (mg) | to bath (ml) | (ml) | (ml) | (mg/L) |
| 1 hr bath | 20 | 85.25 | 10.00 | 240.00 | 250.00 | 170.5 |
| daily | | | 5.00 | 245.00 | 250.00 | 85.3 |
| | | | 2.50 | 247.50 | 250.00 | 42.6 |
| | | | 1.25 | 248.75 | 250.00 | 21.3 |
| Cont. | 500 | 682.03 | 250.00 | 1750.00 | 2000.00 | 170.5 |
| | | | 125.00 | 1875.00 | 2000.00 | 85.3 |
| | | | 62.50 | 1937.50 | 2000.00 | 42.6 |
| | | | 31.25 | 1968.75 | 2000.00 | 21.3 |

### Trojan Egg Inoculation & Bath Treatment

Any dead eggs resulting after disinfection, handling and treatment distribution were replaced with healthy eggs the following day post egg distribution. Trojan eggs from S. diclina petri dishes (FIG. 34) were inoculated at one trojan/well except Treatment G and M (No-sap control) which continued without any pathogen being introduced. Both 1 hr and continuous immersion treatments were introduced after trojan inoculation. Eggs in treatments A, B, C, and D were subject to 1 hr exposure to immersion baths in a separate container. The 6-well plates were carefully removed from its 2 L housing container and submerged in the immersion baths. After 1 hr, the 6-well plates were carefully returned to their original housing container. To mimic the handling stress of other treatments, eggs in treatments F and G were also taken out and replaced back into its container immediately twice at 1 hr interval. The immersion baths were done once a day till study termination. Formalin immersion treatment was conducted in similar way to that of LP34058 except the duration of exposure was only 15 minutes. Eggs in continuous immersion treatments remained uninterrupted in the housing containers.

### Study Termination

Nine days post challenge 100% eggs in treatments H, I, J, and K were dead. Thus, the continuous immersion treatments including controls e.g., L, and M, were terminated by carefully counting number of apparently live eggs, hatched eggs, dead eggs without entanglement, dead eggs entangled to trojan, uninfected eggs and total number of eggs entangled to each trojan. One hour immersion treatments were terminated in a similar way ten days post challenge.

### Statistical Analysis

Parametric data yielded from the study were analyzed by one-way ANOVA followed by Tukey's multiple comparisons test where significant differences were observed. For each analysis, normality of residuals was tested to ensure model fitness.

### Results

### 1 hr Immersion Bath

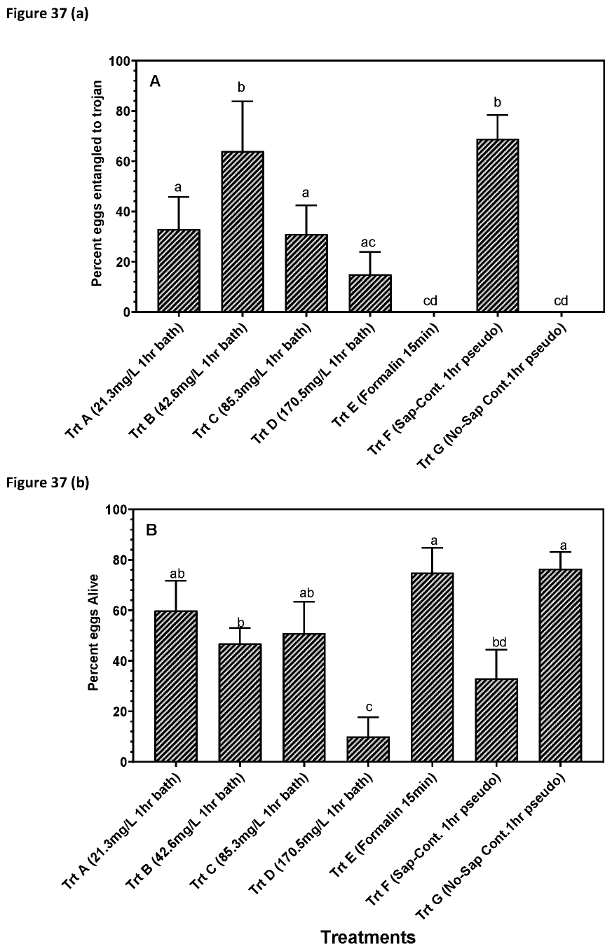

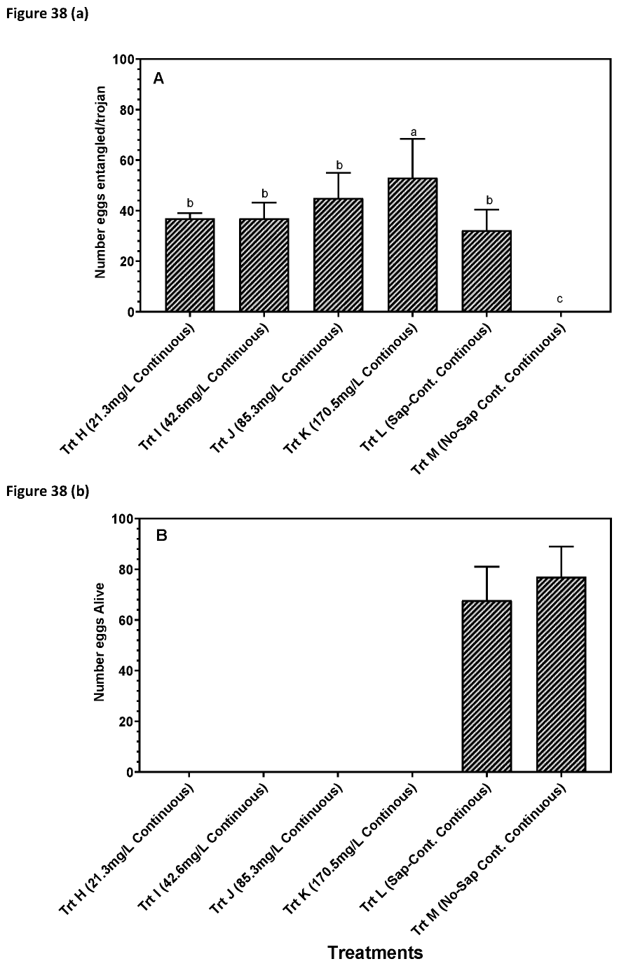

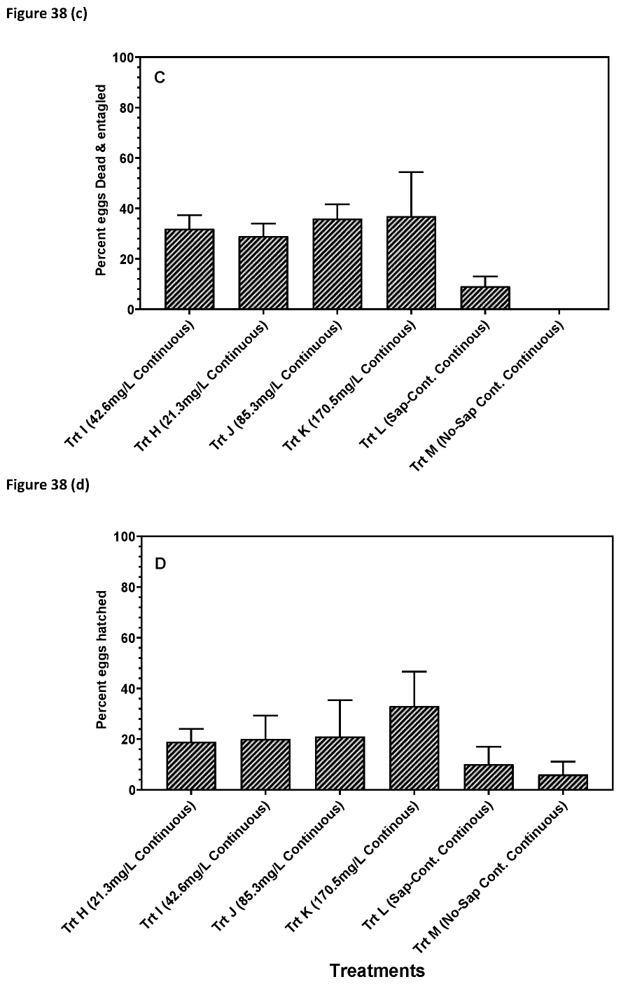

The study compared the efficacy and toxicity of four concentrations of LP34058 immersion treatments on Atlantic salmon eggs challenged with S. diclina. When LP34058 was administered utilizing one hour baths every calendar day for 10 days, a significant reduction in entanglement was observed in A (21.3 mg/L), C (85.3 mg/L), and D (170.5 mg/L) compared to the Sap-control (Trt A, P=0.002; Trt C, P=0.001; Trt D, P<0.0001) while formalin treated eggs did not suffer any sign of saprolegniosis (FIG. 37A; 35A-G). Eggs in treatment B (42.6 mg/L) showed similar entanglements to that of Sap-control (Table 1) which illustrate the lack of protection from LP34058 in this treatment (FIG. 37A; 35B). Survival of eggs between treatment A, B, and C was similar and were comparable to that of No-sap control and formalin treatments (FIG. 37B). However, eggs in sap-control treatment demonstrated significantly higher mortality rate than No-sap control and formalin treatments (Trt F, P<0.0001) which points toward Saprolegnia induced mortality during the study (FIG. 37B). Despite significantly lower rate of entanglement, treatment D (170.5 mg/L) suffered highest mortality revealing a toxic effect of LP34058 at this high concentration (FIG. 35D). In addition, a number of eggs in all treatments were observed to have hatched and died. Although tested eggs were close to hatching (˜385-degree days), treatment D and all continuous LP34058 treatments yielded 20-50% hatching followed by mortality as opposed to no-Sap controls (hatching <15%) which further refer to the toxic effect of LP34058 at high dose or continuous exposure (FIG. 37D, and FIG. 38D). Formalin and No-sap control treatments continued to exhibit lowest hatching compared to rest of the treatments throughout the study period (FIG. 37D).

TABLE 22-3
Average number and average percent of eggs entangled to trojan in the treatments.

| | Chemical | Dose | Average Number | Average Percent |
|---|---|---|---|---|
| Treatment | Treated | (mg/L) | of Eggs Entangled | Eggs Entangled |
| A | LP34058 | 21.3 | 8.25 | 33.00 |
| B | LP34058 | 42.6 | 16.00 | 64.00 |
| C | LP34058 | 85.3 | 7.25 | 31.00 |
| D | LP34058 | 170.5 | 3.75 | 15.00 |
| E | Formalin | 1665 | 0.00 | 0.00 |
| F | Sap-Control | — | 17.25 | 69.00 |
| G | No-Sap Control | — | 0.00 | 0.00 |
| H | LP34058 | 21.3 | 9.25 | 37.00 |
| I | LP34058 | 42.6 | 9.25 | 36.94 |
| J | LP34058 | 85.3 | 11.25 | 45.00 |
| K | LP34058 | 170.5 | 13.25 | 53.00 |
| L | Sap-Control | — | 8.00 | 32.21 |
| M | No-Sap Control | — | 0.00 | 0.00 |

### Continuous Immersion Bath

Continuous immersion treatments were proven to be highly toxic on eggs resulting in 100% mortality of eggs in all LP34058 treatments. Saprolegnia growth was not affected in these continuous immersion treatments despite the toxic effect on eggs. Mycelial entanglements were evident which spread from the trojans into surrounding dead eggs (FIG. 35).

### DISCUSSION

The study demonstrated encouraging results of LP34058 in providing protection against saprolegniosis when administered in 1 hr immersion method. Although the highest dose in 1 hr immersion method was proven to be toxic on eggs, the lowest dose i.e., Trt A (21.3 mg/L) demonstrated promising efficacy in reducing mycelial entanglement and higher survival in eggs compared to the Saprolegnia control treatment. Eggs in Trt B (42.6 mg/L) suffered highest entanglement than the rest of the LP34058 doses which may have resulted due to instability of the active ingredient in this treatment. However, similar survival rates observed among the LP34058 treatments A-C demonstrate the safety of the product on eggs at these lower doses. Despite the protection, all LP34058 treatments suffered some mortalities and henceforth, mycelia grew from trojan to colonize the dead eggs, especially in the continuous immersion treatments. With access to abundant nutrient from the dead eggs, mycelia could have actively deterred any negative effect enabling them to grow and colonize the eggs in presence of LP34058 (Liu et al. 2015). Furthermore, eggs often respond to acute toxicity by hatching early to escape the condition which was evident in the highest dose in 1 hr exposure and continuous treatments further demonstrating the toxic effect of LP34058 at the given doses and exposure method. Nonetheless, it remains unclear whether the mode of action of LP34058 is to actively prevent Saprolegnia from growing, or passively to provide a layer of protection to the eggs since a dose-depended protection was not observed during the study. Therefore, investigations on further lower doses of LP34058 in in vivo and in vitro studies are deemed necessary before the product may be suitable for use in commercial hatchery condition in the treatment of saprolegniosis.

LP34058 when administered in continuous exposure method was found to be highly toxic on the eggs which might be related to the negative effect of the active ingredient in the product. In addition, continuous exposure treatments also yielded higher incident of hatching and larval mortality which could be the outcome of LP34058 toxicity. However, the mechanism of toxicity remains to be fully understood. Large amounts of lipid molecules present in Atlantic salmon eggs allow for essential membrane fluidity and ion exchange during development which might have been affected by the active ingredient in continuous exposure method resulting in the toxicity (Cornet et al. 2021). LP34058 may have contributed to degradation of the chorion layer exposing the premature larvae to mortality.

### Conclusion

The study demonstrated that 1 hr daily exposure of eggs to the lowest LP34058 dose (21.3 mg/L) was more efficacious than the higher doses. However, formalin treatment showed the best results in terms of survival and entanglement. Atlantic salmon eggs were highly susceptible to the toxic effect of LP34058 when administered by continuous immersion method which caused 100% mortality. Despite the toxicity, Saprolegnia mycelia actively colonized dead eggs in these treatments. Therefore, the doses of LP34058 on or below 21.3 mg/L yet high enough to control the Saprolegnia mycelia, in, preferably 1 hr, immersion bath holds the most promising potential against saprolegniosis.

### Example 23—Comparison of Ich and Tetrahymena

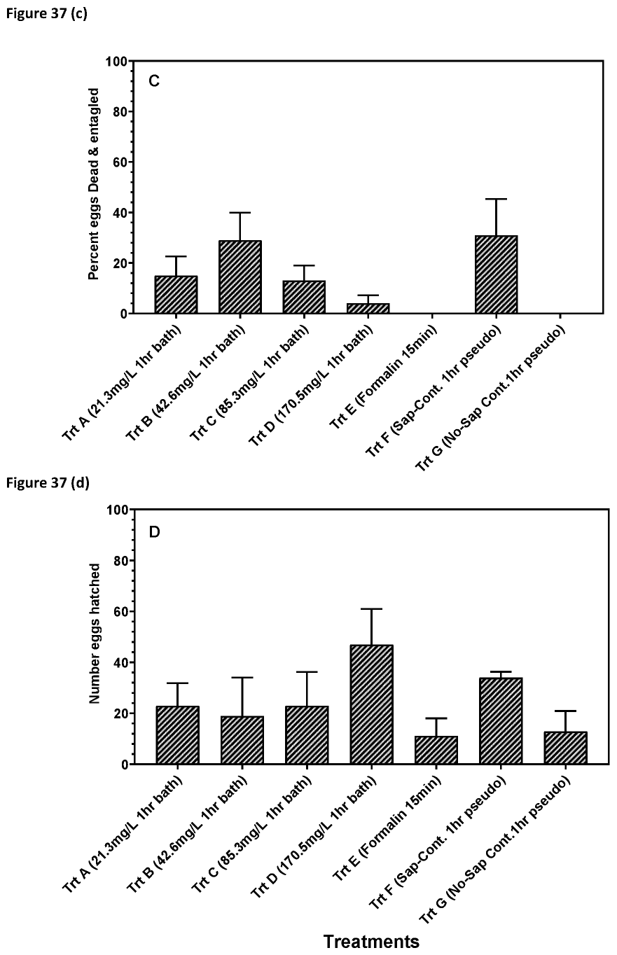

To demonstrate the distinctiveness of two different ciliates, the characteristics of Ich and Tetrahymena were compared:

| | Ich | Tetrahymena |
|---|---|---|
| Life cycle | Dependent on host (e.g., fish) to | Do not need a host to proliferate |
| | proliferate), obligate parasitic | Mainly propagate by binary fission |
| | Four distinct life stages (trophont, | (=division of a single cell into two) |
| | tomont, tomocyst and theront), see | |
| | FIG. 36 for illustrative comparison. | |
| Habitat | Freshwater | Freshwater |
| | Trophont - embedded in fish tissue; | Free swimming in water column |
| | Tomocyst - stuck to surface; Tomont | |
| | and theronts - free swimming | |
| Phylogeny | Ich is relatively closely related to Tetrahymena within the phylum of ciliates | |
| | (“Ciliophora”). The two lineages are estimated to have diverged a little over 500 | |
| | million years ago. In comparison, the human lineages have been estimated to | |
| | diverged from teleost/ray finned fish or lampreys an ancient extant lineage of | |
| | jawless fish (Delsuc et al. 2018) at around 500 million years ago. So even though | |
| | Ich and Tetrahymena at first glance seems relative closely related in the literature, | |
| | it has been a long time since they diverged and evolved from each other. | |
| | Another factor that should be consider is that humans have a generation time of | |
| | several decades while at optimal conditions the generation time for Tetrahymena | |
| | thermophila and Ich in number is 2 hrs and 3-6 days, respectively. | |
| Parasitism | Exclusively parasitic genus | Genus both contain parasitic and non- |
| | | parasitic species |
| Size | From 30-1000 μm wide | Generally, around 30-50 μm wide |

| | |
|---|---|
| Comparison | Tetrahymena thermophila cells were generally more tolerant to copper sulphate, |
| of tolerance | formalin, and malachite green than Ich theronts, and Ich tomonts showed similar |
| to similar | tolerance to T. thermophila. See below table. |
| parasiticides | Toxicity test have shown difference in Roundup-Glyphosate tolerance in |
| | T. thermophila (=1.38 mM), Ich theronts (=0.17 mM) and tomonts (0.34 mM) |

Median lethal concentrations (LC₅₀) of copper sulphate, formalin and
malachite on Ich theronts and T. thermophila, determined using MTS assay

| | | 1 h | | 24 h | |
|---|---|---|---|---|---|
| Treatment | Protozoa | LC₅₀ | 95% CI | LC₅₀ | 95% CI |
| Copper sulphate | Ich theronts | 0.15^{a} | 0.09-0.22 | 0.08^{a} | 0.02-0.14 |
| | T. thermophila | 0.28^{a} | 0.19-0.39 | 0.24^{b} | 0.17-0.32 |
| Formalin | Ich theronts | 10.7^{a} | 3.4-18.1 | 8.5^{a} | 3.9-13.2 |
| | T. thermophila | 15.7^{a} | 9.13-22.4 | 13.7^{a} | 6.4-19.7 |
| Malachite green | Ich theronts | 0.18^{a} | 0.10-0.26 | 0.18^{a} | 0.11-0.27 |
| | T. thermophila | 0.36^{b} | 0.18-0.54 | 0.25^{a} | 0.04-0.46 |

Each value is the mean from four trials, and results are expressed as LC₅₀ and 95% confidence interval (CI). Within a column for each chemical treatment, means followed by the diferent lower case letter are significandy diferent (P < 0.05).

### REFERENCES

de Bruijn I, de Kock M J, de Waard P, van Beek T A, Raaijmakers J M. (2008) Massetolide A biosynthesis in Pseudomonas fluorescens. Journal of Bacteriology. 190(8):2777-89. DOI: 10.1128/JB.01563-07
Liu Y, Rzeszutek E, van der Voort M, Wu C H, Thoen E, Skaar I, et al. (2015) Diversity of Aquatic Pseudomonas Species and Their Activity against the Fish Pathogenic Oomycete Saprolegnia. PLoS ONE. 10(8): e0136241. DOI: 10.1371/journal.pone.0136241
Liu Y, de Bruijn I, Jack A L H, Drynan K, van den Berg A H, Thoen E, et al. (2014) Deciphering microbial landscapes of fish eggs to mitigate emerging diseases. ISME J. 8(10):2002-14. pmid:24671087
Kruijt M, Tran H, Raaijmakers J M. (2009) Functional, genetic and chemical characterization of biosurfactants produced by plant growth-promoting Pseudomonas putida 267. Journal of Applied Microbiology. 107(2):546-56. DOI: 10.1111/j.1365-2672.2009.04244.x
Oni F E, Geudens N, Adiobo A, Omoboye O O, Enow E A, Onyeka J T, Salami A E, De Mot R, Martins J C, Höfte M. (2020) Biosynthesis and antimicrobial activity of Pseudodesmin and Viscosinamide cyclic lipopeptides produced by Pseudomonads associated with the cocoyam rhizosphere. Microorganisms. 8(7):1079. DOI:10.3390/microorganisms8071079
Rokni-Zadeh H, Li W, Yilma E, Sanchez-Rodriguez A, De Mot R. (2013) Distinct lipopeptide production systems for WLIP (white line-inducing principle) in Pseudomonas fluorescens and Pseudomonas putida. Environmental Microbiology Reports. 5(1):160-9. DOI: 10.1111/1758-2229.12015
Jensen H M, Mohammad Karami A, Mathiessen H, A I-Jubury A, Kania P W, Buchmann K (2020) Gill amoebae from freshwater rainbow trout (Oncorhynchus mykiss): In vitro evaluation of antiparasitic compounds against Vannella sp. Journal of Fish Diseases. 43:665-672. DOI: 10.1111/jfd.13162
Trasviña-Moreno A G, Ascencio F, Angulo C, Hutson K S, Aviles-Quevedo A, Inohuye-Rivera R B, Perez-Urbiola J C (2017) Plant extracts as a natural treatment against the fish ectoparasite Neobenedenia sp. (Monogenea: Capsalidae). Journal of Helminthology. DOI: 10.1017/50022149X17001122
Cano I, Taylor N G H, Bayley A, Gunning S, McCullough R, Bateman K, Nowak B F, Paley R K (2019) In vitro gill cell monolayer successfully reproduces in vivo Atlantic salmon host responses to Neoparamoeba perurans infection. Fish & Shellfish Immunology. 86:287-300. DOI: 10.1016/j.fsi.2018.11.029.
OECD (2011), Test No. 201: Freshwater Alga and Cyanobacteria, Growth Inhibition Test, OECD Guidelines for the Testing of Chemicals, Section 2, OECD Publishing, Paris, https://doi.org/10.1787/9789264069923-en.
AAT Bioquest, Inc. (2022, March 14). Quest Graph™ LC50 Calculator. AAT Bioquest. https://www.aatbio.com/tools/lc50-calculator.)
Cornet V, Geay F, Erraud A, Mandiki S N M, Flamion E, Larondelle Y, Rollin X, Kestemont P, 2021. Modulations of lipid metabolism and development of the Atlantic salmon (Salmo salar) fry in response to egg-to-fry rearing conditions. Fish Physiology and Biochemistry 47: 979-997.
Boucher, S. E. and Gillin, F. D. (1990), Excystation of in vitro-derived Giardia lamblia cysts. Infection and Immunity. 58:11. 3516-3522.
Molan, A. L., Liu, Z and De, S. (2009), Effect of pine bark (Pinus radiata) sporulation of coccidian oocysts. Folia Parasitologica. 56:1. 1-5.

1. A method for the killing, inactivating, or inhibiting of one or more harmful blue-green algae or algae capable of causing Harmful Algal Bloom (HAB) in marine, brackish or freshwater environments;
comprising contacting the blue-green algae or algae with an effective amount of a lipopeptide biosurfactant; wherein the blue-green algae or algae is contacted with the lipopeptide biosurfactant in an aqueous solution comprising an effective amount of the lipopeptide biosurfactant to kill, inactivate, or inhibit the blue-green algae or algae.

2. The method of claim 1, wherein the lipopeptide biosurfactant comprises
a) a viscosin or viscosin-like lipopeptide or a derivative thereof;
b) a massetolide or a derivative thereof and/or
c) a putisolvin or a derivative thereof
or any combination thereof

3. The method of any preceding claim, wherein the lipopeptide biosurfactant is isolated from a microbial source, optionally from a bacterium, a fungus or an algae.

4. The method of claim 3, wherein the microbial source is a bacterium, optionally of the genus Pseudomonas fluorescens, optionally Pseudomonas fluorescens strain H6 or the Pseudomonas fluorescens strain SDW1 deposited under deposit number DSMZ-34058.

5. The method of claim 1 to 4, wherein the lipopeptide biosurfactant is a viscosin or viscosin-like lipopeptide or a derivative thereof.

6. The method of claim 5, comprising wherein a viscosin-like lipopeptide or a derivative thereof isolated from Pseudomonas fluorescens strain H6 or DSMZ-34058.

7. The method of claim any preceding claim, wherein the aqueous solution is a marine solution or a freshwater solution or a brackish solution or a hypersaline solution.

8. The method of claim 1 to 7, wherein the algae is selected from the class Dinophyceae, Pelagophyceae, Raphidophyceae, or Prymnesiophyceae or a combination thereof.

9. The method of claim 8, wherein the Dinophyceae is of the order Actiniscales, Amphidiniales, Amphilothales, Blastodiniales, Brachidiniales, Coccidiniales, Dinophysiales, Gloeodiniales, Gonyaulacales, Gymnodiniales, Lophodiniales, Noctilucales, Oxyrrhinales, Peridiniales, Phytodiniales, Prorocentrales, Pyrocystales, Suessiales, Syndiniales, Thoracosphaerales, Torodiniales or Tovelliales or a combination thereof.

10. The method of claim 9, wherein the
a) Gonyaulacales is of i) the family Ostreopsidaceae, optionally of the genus Alexandrium, optionally of the species A. tamarense; optionally of the genus Gambierdiscus, optionally the species G. toxicus, or ii) the family Ceratiaceae, optionally the genus Ceratium;
b) Noctilucales is of the family Noctilucaceae, optionally of the genus Noctiluca; and/or
c) Gymnodiniales is of the family i) Kareniaceae, optionally the genus Karenia; optionally of the species K. brevis; or ii) Gymnodiniaceae, optionally the genus Cochlodinium.

11. The method of claim 8, wherein the Pelagophyceae is of the order Pelagomonadales or Sarcinochrysidales or a combination thereof.

12. The method of claim 11, wherein the Pelagomonadales is of the genus Aureococcus, optionally the species A. anophagefferens.

13. The method of claim 8, wherein the Raphidophyceae is of the order Actinophryida, Chattonellales, Commatiida, or Raphidomonadales or a combination thereof.

14. The method of claim 13, wherein the Chattonellales is of the genus Heterosigma, optionally the species H. akashiwo.

15. The method of claim 8, wherein the Prymnesiophyceae is of the order Coccolithales, Coccosphaerales, Isochrysidales, Phaeocystales, Prymnesiales, Prymnesiophyceae incertae sedis, Syracosphaerales, or Zygodiscales.

16. The method of claim 15, wherein the Prymnesiales is of the genus Prymnesium, optionally the species P. parvum.

17. The method of claim 1, wherein the blue-green algae is of the order Chroococcales, optionally of the family Microcystaceae, optionally of the genus Microcystis.

18. The method of claim 8 to 17, wherein concentration of lipopeptide biosurfactant in the aqueous solution is from 5 μg/mL to 1000 mg/L.

19. The method of claim 18, wherein the effective amount of the lipopeptide biosurfactant is between 0.1 to 1000 mg/L, optionally 0.5 to 500 mg/L, optionally 1 to 100 mg/L, optionally 2 to 50 mg/l, optionally 5 to 25 mg/L.

20. A lipopeptide biosurfactant for use in the treatment of an infection in a subject by one or more harmful blue-green algae or algae capable of causing Harmful Algal Bloom (HAB) in marine, brackish or freshwater environments.

21. The lipopeptide biosurfactant of any preceding claim, wherein the lipopeptide biosurfactant comprises
a) a viscosin or viscosin-like lipopeptide or a derivative thereof;
b) a massetolide or a derivative thereof and/or
c) a putisolvin or a derivative thereof
or any combination thereof

22. The lipopeptide biosurfactant of any preceding claim, wherein the lipopeptide biosurfactant is isolated from a microbial source, optionally from a bacterium, a fungus or an algae.

23. The lipopeptide biosurfactant of claim 22, wherein the microbial source is a bacterium, optionally of the genus Pseudomonas fluorescens, optionally Pseudomonas fluorescens strain H6 or the Pseudomonas fluorescens strain SDW1 deposited under deposit number DSMZ-34058.

24. The lipopeptide biosurfactant claim 23, wherein the lipopeptide biosurfactant is a viscosin or viscosin-like lipopeptide or a derivative thereof.

25. The lipopeptide biosurfactant of claim 24, comprising wherein a viscosin-like lipopeptide or a derivative thereof isolated from Pseudomonas fluorescens strain H6 or DSMZ-34058.

26. The lipopeptide biosurfactant or the method of any preceding claim, wherein the pathogen or pest is contacted with a composition comprising from 5-1000 μg/ml of the lipopeptide biosurfactant.

27. A bacterial isolate for use in the killing, inactivating, or inhibiting of one or more harmful blue-green algae or algae capable of causing Harmful Algal Bloom (HAB) in marine, brackish or freshwater environment;
wherein the bacterial isolate comprises bacteria that produce a lipopeptide surfactant.

28. The bacterial isolate of claim 27 wherein the bacteria produce a massetolide or derivative thereof.

29. The bacterial isolate of claim 27 to 28, wherein the bacteria produce a putisolvin or a derivative thereof.

30. The bacterial isolate of claim 27 to 29, wherein the bacteria comprise Pseudomonas sp. strain H6 or or DSMZ-34058.
